(19) United States
(12) Patent Application Publication
(10) Pub. No.: US 2008/0242694 A1
(43) Pub. Date: Oct. 2, 2008

(54) Amino-substituted heterocycles, compositions thereof, and methods of treatment therewith

(76) Inventors: Neil R. D'Sidocky, Carewood, OH (US); Roy L. Harris, San Diego, CA (US); Sayee G. Hegde, San Diego, CA (US); Robert Hilgraf, San Deigo, CA (US); Margaret A. McCarrick, San Diego, CA (US); Jeffrey A. McKie, San Marcos, CA (US); Deborah S. Mortensen, San Diego, CA (US); Lisa Nadolny, San Diego, CA (US); Sophie M. Perin-Ninkovic, Carlsbad, CA (US); John J. Sapienza, Chula Vista, CA (US); Jonathan L. Wright, San Diego, CA (US)

Correspondence Address: JONES DAY, 222 EAST 41ST ST, NEW YORK, NY, 10017, US

(21) Appl. No.: 11/901,598

(22) Filed: Sep. 17, 2007

Related U.S. Application Data
(60) Provisional application No. 60/845,558, filed on Sep. 18, 2006.

(51) Int. Cl. A61K 31/4375; C07D 417/04; A61P 35/00; A61K 31/4725

(52) U.S. Cl. 514/300; 546/148; 514/307; 546/122

(57) ABSTRACT

Provided herein are Heterocyclic Compounds having the following structure:

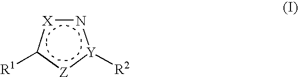

wherein R¹, R², X, Y and Z are as defined herein, compositions comprising an effective amount of a Heterocyclic Compound and methods for treating or preventing cancer, inflammatory conditions, immunological conditions, metabolic conditions and conditions treatable or preventable by inhibition of a kinase pathway comprising administering an effective amount of a Heterocyclic Compound to a patient in need thereof.

This application claims the benefit of U.S. provisional application No. 60/845,558, filed Sep. 18, 2006, which is incorporated by reference herein in its entirety.

### 1. FIELD

Provided herein are certain amino-substituted heterocyclic compounds, compositions comprising an effective amount of such compounds and methods for treating or preventing cancer, inflammatory conditions, immunological conditions, metabolic conditions and conditions treatable or preventable by inhibition of a kinase pathway, comprising administering an effective amount of an amino-substituted heterocyclic compound to a patient in need thereof.

### 2. BACKGROUND

The connection between abnormal protein phosphorylation and the cause or consequence of diseases has been known for over 20 years. Accordingly, protein kinases have become a very important group of drug targets. See Cohen, Nature, 1:309-315 (2002). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001).

The protein kinases are a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharmaceutical Research, 17(11):1345-1353 (2000). Viral infections and the conditions related thereto have also been associated with the regulation of protein kinases. Park et al. Cell 101 (7), 777-787 (2000).

Protein kinases can be divided into broad groups based upon the identity of the amino acid(s) that they target (serine/threonine, tyrosine, lysine, and histidine). For example, tyrosine kinases include receptor tyrosine kinases (RTKs), such as growth factors and non-receptor tyrosine kinases, such as the src kinase family. There are also dual-specific protein kinases that target both tyrosine and serine/threonine, such as cyclin dependent kinases (CDKs) and mitogen-activated protein kinases (MAPKs). A class of thiazole compounds reported to have activity as CDK inhibitors is set forth in U.S. Pat. No. 6,720,427. Any particular cell contains many protein kinases, some of which phosphorylate other protein kinases. Some protein kinases phosphorylate many different proteins, others phosphorylate only a single protein. Not surprisingly, there are numerous classes of protein kinases. Upon receiving a signal, some proteins may also undergo auto-phosphorylation.

The protein tyrosine kinases (PTKs) compose a large family of kinases that regulate cell to cell signals involved in growth, differentiation, adhesion, motility, and death. Robinson et al., Oncogene 19:5548-5557 (2000). Members of the tyrosine kinase include, but are not limited to, Yes, BMX, Syk, EphA1, FGFR3, RYK, MUSK, JAK1 and EGFR. Tyrosine kinases are distinguished into two classes, i.e., the receptor type and non-receptor type tyrosine kinases. Interestingly, the entire of family of tyrosine kinases is quite large—consisting of at least 90 characterized kinases with at least 58 receptor type and at least 32 nonreceptor type kinases comprising at least 30 total subfamilies. Robinson et al., Oncogene 19:5548-5557 (2000). Tyrosine kinases have been implicated in a number of diseases in humans, including diabetes and cancer. Robinson et al. at page 5548. Tyrosine kinases are often involved in most forms of human malignancies and have been linked to a wide variety of congenital syndromes. Robertson et al., Trends Genet. 16:265-271 (2000).

The non-receptor tyrosine kinases represent a group of intracellular enzymes that lack extracellular and transmembrane sequences. Currently, over 32 families of non-receptor tyrosine kinases have been identified. Robinson et al., Oncogene 19:5548-5557 (2000). Examples are Src, Btk, Csk, ZAP70, Kak families. In particular, the Src family of non-receptor tyrosine kinase family is the largest, consisting of Src, Yes, Fyn, Lyn, Lck, Blk, Hck, Fgr and Yrk protein tyrosine kinases. The Src family of kinases have been linked to oncogenesis, cell proliferation and tumor progression. A detailed discussion of non-receptor protein tyrosine kinases is available in Oncogene 8:2025-2031 (1993). Many of these protein tyrosine kinases have been found to be involved in cellular signaling pathways involved in various pathological conditions including but not limited to cancer and hyperproliferative disorders and immune disorders.

The cyclin dependent kinases CDKs represent a group of intracellular enzymes that control progression through the cell cycle and have essential roles in cell proliferation. See Cohen, Nature, 1:309-315 (2002). Examples of CDKs include, but are not limited to, cyclin dependent kinase 2 (CDK2), cyclin dependent kinase 7 (CDK7), cyclin dependent kinase 6 (CDK6) and cell division control 2 protein (CDC2). CDKs have been implicated in the regulation of transitions between different phases of the cell cycle, such as the progression from a quiescent stage in G1 (the gap between mitosis and the onset of DNA replication for a new round of cell division) to S (the period of active DNA synthesis), or the progression from G₂ to M phase, in which active mitosis and cell division occur. See e.g., the articles compiled in Science, vol. 274 (1996), pp. 1643-1677; and Ann. Rev. Cell Dev Biol., vol. 13 (1997), pp. 261-291. CDK complexes are formed through association of a regulatory cyclin subunit (e.g., cyclin A, B1, B2, D1, D2, D3, and E) and a catalytic kinase subunit (e.g., cdc2 (CDK1), CDK2, CDK4, CDK5, and CDK6). As the name implies, CDKs display an absolute dependence on the cyclin subunit in order to phosphorylate their target substrates, and different kinase/cyclin pairs function to regulate progression through specific portions of the cell cycle. CDKs have been implicated in various disease states, including but not limited to, those displaying the cancer phenotype, various neoplastic disorders and in neurological disorders. Hunter, Cell 100:113-127 (2000).

The mitogen activated protein (MAP) kinases participate in the transduction of signals to the nucleus of the cell in response to extracellular stimuli. Examples of MAP kinases include, but are not limited to, mitogen activated protein kinase 3 (MAPK3), mitogen-activated protein kinase 1 (ERK2), mitogen-activated protein kinase 7 (MAPK7), mitogen-activated protein kinase 8 (JNK1), mitogen-activated protein kinase 14 (p38 alpha), mitogen-activated protein kinase 10 (MAPK 10), JNK3 alpha protein kinase, stress-activated protein kinase JNK2 and mitogen-activated protein kinase 14 (MAPK14). MAP kinases are a family of proline-directed serine/threonine kinases that mediate signal transduction from extracellular receptors or heath shock, or UV radiation. See Sridhar et al., Pharmaceutical Research, 17:11 1345-1353 (2000). MAP kinases activate through the phosphorylation of theonine and tyrosine by dual-specificity protein kinases, including tyrosine kinases such as growth factors. Cell proliferation and differentiation have been shown to be under the regulatory control of multiple MAP kinase cascades. See Sridhar et al., Pharmaceutical Research, 17:11 1345-1353 (2000). As such, the MAP kinase pathway plays critical roles in a number of disease states. For example, defects in activities of MAP kinases have been shown to lead to aberrant cell proliferation and carcinogenesis. See Hu et al., Cell Growth Differ. 11:191-200 (2000); and Das et al., Breast Cancer Res. Treat. 40:141 (1996). Moreover, MAP kinase activity has also been implicated in insulin resistance associated with type-2 diabetes. See Virkamaki et al., J. Clin. Invest. 103:931-943 (1999).

The p90 ribosomal S6 kinases (Rsk) are serine/threonine kinases. The Rsk family members function in mitogen-activated cell growth and proliferation, differentiation, and cell survival. Examples of members of the Rsk family of kinases include, but are not limited to, ribosomal protein S6 kinase, 90 kDa, polypeptide 2 (Rsk3), ribosomal protein S6 kinase, 90 kDa, polypeptide 6 (Rsk4), ribosomal protein S6 kinase, 90 kDa, polypeptide 3 (Rsk2) and ribosomal protein S6 kinase, 90 kDa, polypeptide 1 (Rsk1/p90Rsk). The Rsk family members are activated by extracellular signal-related kinases 1/2 and phosphoinositide-dependent protein kinase 1. Frodin and Gammeltoft, Mol. Cell. Endocrinol. 151:65-77 (1999). Under basal conditions, RSK kinases are localized in the cytoplasm of cells and upon stimulation by mitogens, the activated (phosphorylated by extracellular-related kinase) RSK transiently translocates to the plasma membrane where they become fully activated. The fully activated RSK phosphorylates substrates that are involved in cell growth, proliferation, differentiation, and cell survival. Richards et al., Curr. Biol. 9:810-820 (1999); Richards et al., Mol. Cell. Biol. 21:7470-7480 (2001). RSK signaling pathways have also been associated with the modulation of the cell cycle. Gross et al., J. Biol. Chem. 276(49): 46099-46103 (2001). Current data suggests that small molecules that inhibit Rsk may be useful therapeutic agents for the prevention and treatment of cancer and inflammatory diseases.

Akt/protein kinase B (PKB) is a serine/threonine protein kinase which controls a number of different cellular responses. Toker et al., Cancer Res. 66(8):3963-3966 (2006). Akt increases cell survival in a PI3K-dependent manner and, accordingly, is a target for antineoplastic therapies. Dudek et al., Science 275:661-665 (1997). Indeed, several laboratories have reported increased Akt activity in tumors of the breast, prostate, ovary and pancreas. Altomare et al., Oncogene 24:7455-7464 (2005). These studies provide overwhelming evidence that efficient signaling through the PI3K/Akt pathway promotes growth and survival, and that genetic perturbation of this pathway will increase the survival of cancer cells that would normally undergo apoptosis. Toker et al., Cancer Res. 66(8):3963-3966 (2006). Certain thiadiazole compounds have been reported in International Publication No. WO 2006/044860 as being useful for treating diseases mediated by PKB, however, there is still a need in the art for compounds able to modulate Akt/PKB activity.

Members of the checkpoint protein kinase family are serine/threonine kinases that play an important role in cell cycle progression. Examples of members of the checkpoint family include, but are not limited to, CHK1 and CHK2. Checkpoints are control systems that coordinate cell cycle progression by influencing the formation, activation and subsequent inactivation of the cyclin-dependent kinases. Checkpoints prevent cell cycle progression at inappropriate times, maintain the metabolic balance of cells while the cell is arrested, and in some instances can induce apoptosis (programmed cell death) when the requirements of the checkpoint have not been met. See e.g., O'Connor, Cancer Surveys, 29: 151-182 (1997); Nurse, Cell, 91: 865-867 (1997); Hartwell et al., Science, 266: 1821-1828 (1994); Hartwell et al., Science, 246: 629-634 (1989). Members of the checkpoint family of kinases have been implicated in cell proliferative disorders, cancer phenotypes and other diseases related to DNA damage and repair. Kohn, Mol. Biol. Cell 10:2703-2734 (1999); Ohi and Gould, Curr. Opin. Cell Biol. 11:267-273 (1999); Peng, et al., Science 277:1501-1505 (1997).

Aurora kinases are a family of multigene mitotic serine-threonine kinases that functions as a class of novel oncogenes. These kinases comprise aurora-A and aurora-B members. Aurora kinases are hyperactivated and/or over-expressed in several solid tumors including but not limited to, breast, ovary, prostate, pancreas, and colorectal cancers. In particular aurora-A is a centrosome kinase that plays an important role cell cycle progression and cell proliferation. Aurora-A is located in the 20q13 chromosome region that is frequently amplified in several different types of malignant tumors such as colorectal, breast and bladder cancers. There is also a high correlation between aurora-A and high histo-prognostic grade aneuploidy, making the kinase a potential prognostic vehicle. Inhibition of aurora kinase activity could help to reduce cell proliferation, tumor growth and potentially tumorigenesis. A detailed description of aurora kinase function is reviewed in Oncogene 21:6175-6183 (2002).

The Rho-associated coiled-coil-containing protein serine/threonine kinases ROCK-I and ROCK-II are thought to play a major role in cytoskeletal dynamics by serving as downstream effectors of the Rho/Rac family of cytokine- and growth factor-activated small GTPases. ROCKs phosphorylate various substrates, including, but not limited to, myosin light chain phosphatase, myosin light chain, ezrin-radixin-moesin proteins and LIM (for Lin11, Isl1 and Mec3) kinases. ROCKs also mediate the formation of actin stress fibers and focal adhesions in various cell types. ROCKs have an important role in cell migration by enhancing cell contractility. They are required for tail retraction of monocytes and cancer cells, and a ROCK inhibitor has been used to reduce tumor-cell dissemination in vivo. Recent experiments have defined new functions of ROCKs in cells, including centrosome positioning and cell-size regulation, which might contribute to various physiological and pathological states. See Nature Reviews Mol. Cell. Biol. 4, 446-456 (2003). The ROCK family members are attractive intervention targets for a variety of pathologies, including cancer and cardiovascular disease. For example, Rho kinase inhibitors can be useful therapeutic agents for hypertension, angina pectoris, and asthma. Furthermore, Rho is expected to play a role in peripheral circulation disorders, arteriosclerosis, inflammation, and autoimmune disease and as such, is a useful target for therapy.

The 70 kDa ribosomal S6 kinase (p70S6K) is activated by numerous mitogens, growth factors and hormones. Activation of p70S6K occurs through phosphorylation at a number of sites and the primary target of the activated kinase is the 40S ribosomal protein S6, a major component of the machinery involved in protein synthesis in mammalian cells. In addition to its involvement in regulating translation, p70S6K activation has been implicated in cell cycle control, neuronal cell differentiation, regulation of cell motility and a cellular response that is important in tumor metastases, immunity and tissue repair. Modulation of p70S6 kinase activity may have therapeutic implications in disorders such as cancer, inflammation, and various neuropathies. A detailed discussion of p70S6K kinases can be found in Prog. Cell Cycle Res. 1:21-32 (1995), and Immunol Cell Biol. 78(4):447-51 (2000).

Glycogen synthase kinase 3 (GSK-3) is a ubiquitously expressed constitutively active serine/threonine kinase that phosphorylates cellular substrates and thereby regulates a wide variety of cellular functions, including development, metabolism, gene transcription, protein translation, cytoskeletal organization, cell cycle regulation, and apoptosis. GSK-3 was initially described as a key enzyme involved in glycogen metabolism, but is now known to regulate a diverse array of cell functions. Two forms of the enzyme, GSK-3α and GSK-3β, have been previously identified. The activity of GSK-3β is negatively regulated by protein kinase B/Akt and by the Wnt signaling pathway. Small molecules inhibitors of GSK-3 may, therefore, have several therapeutic uses, including the treatment of neurodegenerative diseases, diabetes type II, bipolar disorders, stroke, cancer, and chronic inflammatory disease. Reviewed in Role of glycogen synthase kinase-3 in cancer: regulation by Wnts and other signaling pathways (Adv Cancer Res.; 84:203-29, 2002); Glycogen synthase kinase 3 (GSK-3) inhibitors as new promising drugs for diabetes, neurodegeneration, cancer, and inflammation (Med Res Rev.; 22(4):373-84, 2002); Role of glycogen synthase kinase-3 in the phosphatidylinositol 3-Kinase/Akt cell survival pathway. (J. Biol. Chem., 273(32):19929-32, 1998).

Because protein kinases regulate nearly every cellular process, including metabolism, cell proliferation, cell differentiation, and cell survival, they are attractive targets for therapeutic intervention for various disease states. For example, cell-cycle control and angiogenesis, in which protein kinases play a pivotal role are cellular processes associated with numerous disease conditions such as but not limited to cancer, inflammatory diseases, abnormal angiogenesis and diseases related thereto, atherosclerosis, macular degeneration, diabetes, obesity, and pain.

Protein kinases have become attractive targets for the treatment of cancers. Fabbro et al., Pharmacology & Therapeutics 93:79-98 (2002). It has been proposed that the involvement of protein kinases in the development of human malignancies may occur by: (1) genomic rearrangements (e.g., BCR-ABL in chronic myelogenous leukemia), (2) mutations leading to constitutively active kinase activity, such as acute myelogenous leukemia and gastrointestinal tumors, (3) deregulation of kinase activity by activation of oncogenes or loss of tumor suppressor functions, such as in cancers with oncogenic RAS, (4) deregulation of kinase activity by over-expression, as in the case of EGFR and (5) ectopic expression of growth factors that can contribute to the development and maintenance of the neoplastic phenotype. Fabbro et al., Pharmacology & Therapeutics 93:79-98 (2002).

Certain cancers are associated with angiogenesis. Angiogenesis is the growth of new capillary blood vessels from pre-existing vasculature. Risau, W., Nature 386:671-674 (1997). It has been shown that protein kinases can contribute to the development and maintenance of the neoplastic phenotype. Fabbro et al., Pharmacology & Therapeutics 93:79-98 (2002). For example, VEGF A-D and their four receptors have been implicated in phenotypes that involve neovascularization and enhanced vascular permeability, such as tumor angiogenesis and lymphangiogenesis. Matter, A., Drug Discov. Today 6:1005-1023 (2001).

Cardiovascular disease (“CVD”) accounts for nearly one quarter of total annual deaths worldwide. Vascular disorders such as atherosclerosis and restenosis result from dysregulated growth of the vessel walls and the restriction of blood flow to vital organs. Various kinase pathways, e.g. JNK, are activated by atherogenic stimuli and regulated through local cytokine and growth factor production in vascular cells. Yang et al., Immunity 9:575 (1998). Ischemia and ischemia coupled with reperfusion in the heart, kidney or brain result in cell death and scar formation, which can ultimately lead to congestive heart failure, renal failure or cerebral dysfunction. In organ transplantation, reperfusion of previously ischemic donor organs results in acute leukocyte-mediated tissue injury and delay of graft function. Ischemia and reperfusion pathways are mediated by various kinases. For example, the JNK pathway has been linked to leukocyte-mediated tissue damage. Li et al., Mol. Cell. Biol. 16:5947-5954 (1996). Finally, enhanced apoptosis in cardiac tissues has also been linked to kinase activity. Pombo et al., J. Biol. Chem. 269:26546-26551 (1994).

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on multiple kinases or multiple kinase pathways.

It has therefore been suggested that due to the complexity of intracellular signaling cascades of protein kinase pathways, agents that affect multiple pathways simultaneously may be required for meaningful clinical activity. Indeed, it is known that some kinase drugs, such as Gleevec®, do target several kinases at once. Gleevec® primarily targets a mutant fusion protein containing the abl kinase, which is created by a 9:22 chromosomal translocation event; Gleevec® also targets c-kit, a tyrosine kinase implicated in gastrointestinal stromal tumors (GIST). However, in recent clinical trials, patients have developed resistance to Gleevec® or have shown incomplete response to treatment.

Accordingly, there remains a need for new kinase modulators.

Citation or identification of any reference in Section 2 of this application is not to be construed as an admission that the reference is prior art to the present application.

### 3. SUMMARY

Provided herein are compounds having the following formula (I):

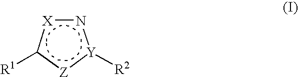

and pharmaceutically acceptable salts, polymorphs, clathrates, solvates, hydrates, stereoisomers and prodrugs thereof, wherein R¹, R², X, Y and Z are as defined herein.

Compounds of formula (I), or a pharmaceutically acceptable salt, clathrate, solvate, hydrate, stereoisomer or prodrug thereof (each being referred to herein as a “Heterocyclic Compound”), are useful for treating or preventing cancer, inflammatory conditions, immunological conditions, metabolic conditions and conditions treatable or preventable by inhibition of a kinase pathway, in one embodiment, the Akt pathway.

Further provided herein are compositions comprising an effective amount of a Heterocyclic Compound and compositions comprising an effective amount of a Heterocyclic Compound and a pharmaceutically acceptable carrier or vehicle. The compositions are useful for treating or preventing cancer, inflammatory conditions, immunological conditions, metabolic conditions and conditions treatable or preventable by inhibition of a kinase pathway, in one embodiment, the Akt pathway.

Further provided herein are methods for treating or preventing cancer, inflammatory conditions, immunological conditions, metabolic conditions and conditions treatable or preventable by inhibition of a kinase pathway, in one embodiment, the Akt pathway, comprising administering an effective amount of a Heterocyclic Compound to a patient in need of the treating or preventing.

In one embodiment, the Heterocyclic Compound targets two or more of the following: kinases from the src kinase family, kinases from the Rsk kinase family, kinases from the CDK family, kinases from the MAPK kinase family, serine/threonine kinases and tyrosine kinases such as Fes, Lyn, and Syk kinases. The agent may target two or more kinases of the same family, or may target kinases representing two or more kinase families or classes.

The present embodiments can be understood more fully by reference to the detailed description and examples, which are intended to exemplify non-limiting embodiments.

### 4. DETAILED DESCRIPTION

### 4.1 Definitions

A “C₁₋₈alkyl” group is a saturated straight chain or branched non-cyclic hydrocarbon having from 1 to 8 carbon atoms. Representative —(C₁₋₈alkyls) include -methyl, -ethyl, -n-propyl, -n-butyl, -n-pentyl, -n-hexyl, -n-heptyl and -n-octyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. A —(C₁₋₈alkyl) group can be substituted or unsubstituted.

An “aminoalkyl” group is a C₁₋₈alkyl group wherein one or more hydrogen atoms is replaced with a —NH₂, —NHR or —NR₂ group, wherein each R is independently an aryl group or a C₁₋₈alkyl group as defined above, wherein each aryl or C₁₋₈alkyl group can be optionally substituted.

An “alkylamino” group is a —NHR or —NR₂ group, wherein each R is independently a C₁₋₈alkyl group as defined above, wherein each C₁₋₈alkyl group can be optionally substituted.

A “halogen” is fluorine, chlorine, bromine or iodine.

An “aryl” group is an unsaturated aromatic carbocyclic group of from 6 to 14 carbon atoms having a single ring (e.g., phenyl) or multiple condensed rings (e.g., naphthyl or anthryl). Particular aryls include phenyl, biphenyl, naphthyl and the like. An aryl group can be substituted or unsubstituted.

A “C₃₋₁₀heteroaryl” group is an aryl ring system having one to four heteroatoms (e.g., O, S or N) as ring atoms in a heteroaromatic ring system, wherein the remainder of the atoms are carbon atoms. Suitable heteroatoms include oxygen, sulfur and nitrogen. In certain embodiments, the heterocyclic ring system is monocyclic or bicyclic. Non-limiting examples include aromatic groups selected from the following:

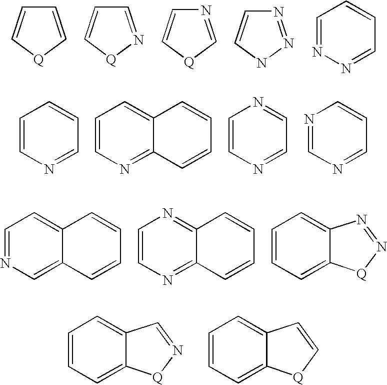

wherein Q is CH₂, CH═CH, O, S or NH. Further representative examples of C₃₋₁₀heteroaryl groups include, but are not limited to, benzofuranyl, benzothienyl, indolyl, benzopyrazolyl, coumarinyl, furanyl, isothiazolyl, imidazolyl, isoxazolyl, thiazolyl, triazolyl, tetrazolyl, thiophenyl, pyrimidinyl, isoquinolinyl, quinolinyl, pyridinyl, pyrrolyl, pyrazolyl, 1H-indolyl, 1H-indazolyl, benzo[d]thiazolyl and pyrazinyl. Further representative examples of heteroaryl groups include those of the compounds disclosed herein. C₃₋₁₀heteroaryls can be bonded at any ring atom (i.e., at any carbon atom or heteroatom of the heteroaryl ring). A C₃₋₁₀heteroaryl can be substituted or unsubstituted.

When the groups described herein are said to be “substituted or unsubstituted,” when substituted, they may be substituted with one or more of any substituent. Examples of substituents are those found in the exemplary compounds and embodiments disclosed herein, as well as halo (e.g., chloro, iodo, bromo, or fluoro); C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; carbamoyl; carbamate; acetal; urea; thiocarbonyl; sulfonyl; sulfonamide; ketone; aldehyde; ester; acetyl; acetoxy; oxygen (═O); haloalkyl (e.g., trifluoromethyl); substituted aminoacyl and aminoalkyl; carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, furanyl, or thiazinyl); carbocyclic or heterocyclic, monocyclic or fused or non-fused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl); amino (primary, secondary, or tertiary); —O-lower alkyl; —O-aryl; aryl; aryl-lower alkyl; CO₂CH₃; CONH₂; OCH₂CONH₂; NH₂; N(C₁₋₄alkyl)₂; NHC(O)C₁₋₄alkyl; SO₂NH₂; SO₂Cl₄alkyl; OCHF₂; CF₃; OCF₃; and such moieties may also be optionally substituted by a fused-ring structure or bridge, for example —OCH₂O— or —O-lower alkylene-O—. These substituents may optionally be further substituted with a substituent selected from such groups.

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770 (1996)).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Heterocyclic Compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “polymorph(s)” and related terms herein refer to solid forms of the Heterocyclic Compounds having different physical properties as a result of the order of the molecules in the crystal lattice. The differences in physical properties exhibited by solid forms affect pharmaceutical parameters such as storage stability, compressibility and density (important in formulation and product manufacturing), and dissolution rates (an important factor in determining bioavailability). Differences in stability can result from changes in chemical reactivity (e.g., differential oxidation, such that a dosage form discolors more rapidly when comprised of one solid form than when comprised of another solid form) or mechanical changes (e.g., tablets crumble on storage as a kinetically favored polymorph converts to thermodynamically more stable solid form) or both (e.g., tablets of one solid form are more susceptible to breakdown at high humidity). As a result of solubility/dissolution differences, in the extreme case, some solid form transitions may result in lack of potency or, at the other extreme, toxicity. In addition, the physical properties of the crystal may be important in processing, for example, one solid form might be more likely to form solvates or might be difficult to filter and wash free of impurities (i.e., particle shape and size distribution might be different between one solid form relative to the other).

As used herein and unless otherwise indicated, the term “clathrate” means a Heterocyclic Compound, or a salt thereof, in the form of a crystal lattice that contains spaces (e.g., channels) that have a guest molecule (e.g., a solvent or water) trapped within or a crystal lattice wherein a Heterocyclic Compound is a guest molecule.

As used herein and unless otherwise indicated, the term “hydrate” means a Heterocyclic Compound, or a salt thereof, that further includes a stoichiometric or non-stoichiometric amount of water bound by non-covalent intermolecular forces.

As used herein and unless otherwise indicated, the term “solvate” means a Heterocyclic Compound, or a salt thereof, that further includes a stoichiometric or non-stoichiometric amount of a solvent bound by non-covalent intermolecular forces.

As used herein and unless otherwise indicated, the term “prodrug” means a Heterocyclic Compound derivative that can hydrolyze, oxidize, or otherwise react under biological conditions (in vitro or in vivo) to provide an active compound, particularly a Heterocyclic Compound. Examples of prodrugs include, but are not limited to, derivatives and metabolites of a Heterocyclic Compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a Heterocyclic Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Heterocyclic Compounds can have chiral centers and can occur as racemates, individual enantiomers or diastereomers, and mixtures thereof. All such isomeric forms are included within the embodiments disclosed herein, including mixtures thereof.

Various Heterocyclic Compounds contain one or more chiral centers, and can exist as racemic mixtures of enantiomers, mixtures of diastereomers or enantiomerically or optically pure compounds. The use of stereomerically pure forms of such Heterocyclic Compounds, as well as the use of mixtures of those forms are encompassed by the embodiments disclosed herein. For example, mixtures comprising equal or unequal amounts of the enantiomers of a particular Heterocyclic Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the Heterocyclic Compounds can include E and Z isomers, or a mixture thereof, and cis and trans isomers or a mixture thereof. In certain embodiments, the Heterocyclic Compounds are isolated as either the E or Z isomer. In other embodiments, the Heterocyclic Compounds are a mixture of the E and Z isomers.

The term “effective amount” in connection with an Heterocyclic Compound can mean an amount capable of treating or preventing a disease disclosed herein, such as cancer, inflammatory conditions, immunological conditions, metabolic conditions or conditions treatable or preventable by inhibition of a kinase pathway, in one embodiment, the Akt pathway.

The term “patient” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### 4.2 Heterocyclic Compounds

Provided herein are Heterocyclic Compounds having the following formula (I):

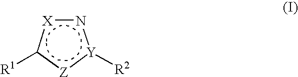

and pharmaceutically acceptable salts, polymorphs, clathrates, solvates, hydrates, stereoisomers, enantiomers and prodrugs thereof,

wherein:

R¹ is substituted or unsubstituted C₃₋₁₀heteroaryl;

R² is substituted or unsubstituted C₁₋₈alkyl, substituted or unsubstituted aminoalkyl or substituted or unsubstituted alkylamino;

X is N or CH;

Y is N or C; and

Z is S, NH or CH.

In one embodiment, the Heterocyclic Compounds of formula (I) are those wherein Y is C.

In one embodiment, the Heterocyclic Compounds of formula (I) are those wherein Y is N.

In another embodiment, the Heterocyclic Compounds of formula (I) are those wherein X is N.

In another embodiment, the Heterocyclic Compounds of formula (I) are those wherein Z is S.

In another embodiment, the Heterocyclic Compounds of formula (I) are those wherein X is N and Y is N.

In another embodiment, the Heterocyclic Compounds of formula (I) are those wherein X is N, Y is C and Z is S.

In another embodiment, the Heterocyclic Compounds of formula (I) are those wherein X is N, Y is N and Z is C.

In another embodiment, the Heterocyclic Compounds of formula (I) are those wherein R¹ is substituted or unsubstituted isoquinoline.

In another embodiment, the Heterocyclic Compounds of formula (I) are those wherein R¹ is substituted or unsubstituted naphthyridine.

In another embodiment, the Heterocyclic Compounds of formula (I) are those wherein R¹ is substituted or unsubstituted indazole.

In another embodiment, the Heterocyclic Compounds of formula (I) are those wherein R¹ is not pyridine.

In another embodiment, the Heterocyclic Compounds of formula (I) are those wherein R² is substituted or unsubstituted C₁₋₈alkyl.

In another embodiment, the Heterocyclic Compounds of formula (I) are those wherein R² is substituted or unsubstituted aminoalkyl.

In another embodiment, the Heterocyclic Compounds of formula (I) are those wherein R² is substituted or unsubstituted alkylamino.

In another embodiment, the Heterocyclic Compounds of formula (I) are those wherein R² is —NHCH(CH₂NH₂)(CH₂C₆H₅).

In another embodiment, the Heterocyclic Compounds of formula (I) are those wherein R² is —(CH₂)₀₋₃C(C₁₋₈alkyl)((CH₂)₀₋₃NH₂)((CH₂)₀₋₃aryl or C₃₋₁₀heteroaryl).

In another embodiment, the Heterocyclic Compounds of formula (I) are those wherein R² is —(CH₂)₀₋₃CH((CH₂)₀₋₃NH₂)((CH₂)₀₋₃aryl or C₃₋₁₀heteroaryl).

In a further embodiment, provided herein are Heterocyclic Compounds having the following formula (II):

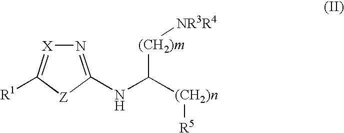

and pharmaceutically acceptable salts, polymorphs, clathrates, solvates, hydrates, stereoisomers, enantiomers and prodrugs thereof,

wherein:

X is N or CH;

Z is NH or S;

R¹ is substituted or unsubstituted C₃₋₁₀heteroaryl;

R³ and R⁴ are independently H, substituted or unsubstituted C₁₋₈alkyl, substituted or unsubstituted aryl or substituted or unsubstituted C₃₋₁₀heteroaryl;

R⁵ is substituted or unsubstituted aryl or substituted or unsubstituted C₃₋₁₀heteroaryl;

m is an integer from 1-3; and

n is an integer from 0-3.

In one embodiment, the Heterocyclic Compounds of formula (II) are those wherein X is N.

In another embodiment, the Heterocyclic Compounds of formula (II) are those wherein Z is S.

In another embodiment, the Heterocyclic Compounds of formula (II) are those wherein Z is N and Z is S.

In another embodiment, the Heterocyclic Compounds of formula (II) are those wherein R¹ is substituted or unsubstituted isoquinoline.

In another embodiment, the Heterocyclic Compounds of formula (II) are those wherein R¹ is substituted or unsubstituted indazole.

In another embodiment, the Heterocyclic Compounds of formula (II) are those wherein R¹ is substituted or unsubstituted naphthyridine.

In another embodiment, the Heterocyclic Compounds of formula (II) are those wherein R¹ is not substituted or unsubstituted pyridine.

In another embodiment, the Heterocyclic Compounds of formula (II) are those wherein R¹ is not substituted or unsubstituted indazole.

In another embodiment, the Heterocyclic Compounds of formula (II) are those wherein R¹ is not substituted or unsubstituted 1H-pyrazolo[3,4-c]pyridine.

In another embodiment, the Heterocyclic Compounds of formula (II) are those wherein R³ and R⁴ are H.

In another embodiment, the Heterocyclic Compounds of formula (II) are those wherein R⁵ is substituted or unsubstituted phenyl.

In another embodiment, the Heterocyclic Compounds of formula (II) are those wherein n is an integer from 1-3.

In another embodiment, the Heterocyclic Compounds of formula (II) are those wherein n is an integer from 2-3.

In a further embodiment, provided herein are Heterocyclic Compounds having the following formula (IIa):

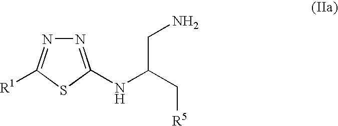

and pharmaceutically acceptable salts, polymorphs, clathrates, solvates, hydrates, stereoisomers, enantiomers and prodrugs thereof,

wherein:

R¹ is substituted or unsubstituted C₃₋₁₀heteroaryl; and

R⁵ is substituted or unsubstituted aryl or substituted or unsubstituted C₃₋₁₀heteroaryl.

In one embodiment, the Heterocyclic Compounds of formula (IIa) are those wherein R¹ is substituted or unsubstituted isoquinoline.

In another embodiment, the Heterocyclic Compounds of formula (IIa) are those wherein R¹ is substituted or unsubstituted indazole.

In another embodiment, the Heterocyclic Compounds of formula (IIa) are those wherein R¹ is substituted or unsubstituted naphthyridine.

In another embodiment, the Heterocyclic Compounds of formula (IIa) are those wherein R¹ is not substituted or unsubstituted pyridine.

In another embodiment, the Heterocyclic Compounds of formula (IIa) are those wherein R¹ is not substituted or unsubstituted indazole.

In another embodiment, the Heterocyclic Compounds of formula (IIa) are those wherein R¹ is not substituted or unsubstituted 1H-pyrazolo[3,4-c]pyridine.

In another embodiment, the Heterocyclic Compounds of formula (IIa) are those wherein R⁵ is substituted or unsubstituted phenyl.

In a further embodiment, provided herein are Heterocyclic Compounds having the following formula (III):

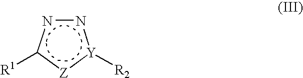

and pharmaceutically acceptable salts, polymorphs, clathrates, solvates, hydrates, stereoisomers, enantiomers and prodrugs thereof,

wherein:

Y is N or C;

Z is CH or S;

R¹ is substituted or unsubstituted C₃₋₁₀heteroaryl; and

R² is substituted or unsubstituted C₁₋₈ alkyl.

In one embodiment, the Heterocyclic Compounds of formula (III) are those wherein Y is N.

In another embodiment, the Heterocyclic Compounds of formula (III) are those wherein Y is C.

In another embodiment, the Heterocyclic Compounds of formula (III) are those wherein Z is CH.

In another embodiment, the Heterocyclic Compounds of formula (III) are those wherein Z is S.

In another embodiment, the Heterocyclic Compounds of formula (III) are those wherein Y is N and Z is CH.

In another embodiment, the Heterocyclic Compounds of formula (III) are those wherein Y is C and Z is S.

In another embodiment, the Heterocyclic Compounds of formula (III) are those wherein R¹ is substituted or unsubstituted isoquinoline.

In another embodiment, the Heterocyclic Compounds of formula (III) are those wherein R¹ is substituted or unsubstituted indazole.

In another embodiment, the Heterocyclic Compounds of formula (III) are those wherein R¹ is substituted or unsubstituted naphthyridine.

In another embodiment, the Heterocyclic Compounds of formula (III) are those wherein R¹ is not pyridine.

In another embodiment, the Heterocyclic Compounds of formula (III) are those wherein R² is aminoalkyl.

In another embodiment, the Heterocyclic Compounds of formula (III) are those wherein R² is —(CH₂)₀₋₃C(C₁₋₆alkyl)((CH₂)₀₋₃NH₂)((CH₂)₀₋₃aryl or C₃₋₁₀heteroaryl).

In another embodiment, the Heterocyclic Compounds of formula (III) are those wherein R² is —(CH₂)₀₋₃CH((CH₂)₀₋₃NH₂)((CH₂)₀₋₃aryl or C₃₋₁₀heteroaryl)

In a further embodiment, provided herein are Heterocyclic Compounds having the following formula (IIIa):

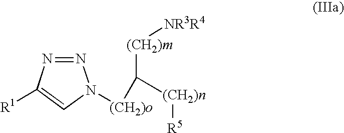

and pharmaceutically acceptable salts, polymorphs, clathrates, solvates, hydrates, stereoisomers, enantiomers and prodrugs thereof,

wherein:

R¹ is substituted or unsubstituted C₃₋₁₀heteroaryl;

R³ and R⁴ are independently H, substituted or unsubstituted C₁₋₈alkyl, substituted or unsubstituted aryl or substituted or unsubstituted C₃₋₁₀heteroaryl;

R⁵ is substituted or unsubstituted aryl or substituted or unsubstituted C₃₋₁₀heteroaryl;

m is an integer from 0-3;

n is an integer from 0-3; and

o is an integer from 0-3.

In one embodiment, the Heterocyclic Compounds of formula (IIa) are those wherein R¹ is substituted or unsubstituted isoquinoline.

In another embodiment, the Heterocyclic Compounds of formula (IIa) are those wherein R¹ is substituted or unsubstituted indazole.

In another embodiment, the Heterocyclic Compounds of formula (IIa) are those wherein R¹ is substituted or unsubstituted naphthyridine.

In another embodiment, the Heterocyclic Compounds of formula (IIIa) are those wherein R¹ is not pyridine.

In another embodiment, the Heterocyclic Compounds of formula (IIIa) are those wherein R³ and R⁴ are H.

In another embodiment, the Heterocyclic Compounds of formula (IIIa) are those wherein R⁵ is substituted or unsubstituted phenyl.

In a further embodiment, provided herein are Heterocyclic Compounds having the following formula (IIIb):

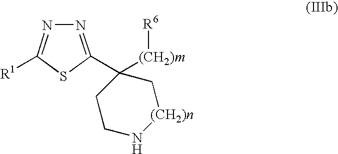

and pharmaceutically acceptable salts, polymorphs, clathrates, solvates, hydrates, stereoisomers, enantiomers and prodrugs thereof,

wherein:

R¹ is substituted or unsubstituted C₃₋₁₀heteroaryl;

R⁶ is substituted or unsubstituted aryl or substituted or unsubstituted C₃₋₁₀heteroaryl;

m is an integer from 1-3; and

n is an integer from 0-2.

In one embodiment, the Heterocyclic Compounds of formula (IIIb) are those wherein R¹ is substituted or unsubstituted isoquinoline.

In another embodiment, the Heterocyclic Compounds of formula (IIIb) are those wherein R⁶ is phenyl.

In another embodiment, the Heterocyclic Compounds of formula (IIIb) are those wherein m is 1.

In another embodiment, the Heterocyclic Compounds of formula (IIIb) are those wherein n is 1.

In another embodiment, the Heterocyclic Compounds of formula (IIIb) are those wherein m is 1 and n is 1.

Representative Heterocyclic Compounds are set forth in Table 1, below.

TABLE 1
Compound

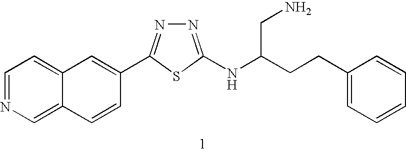

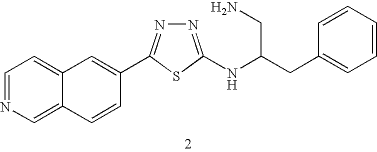

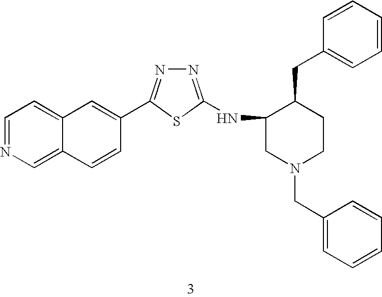

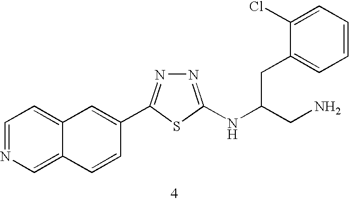

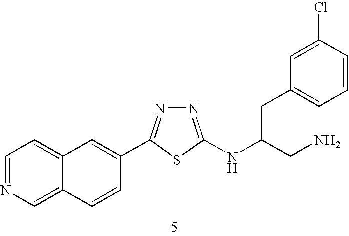

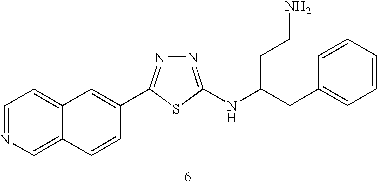

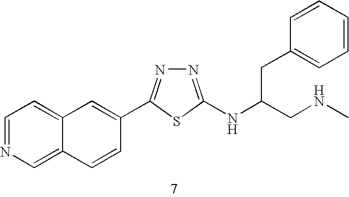

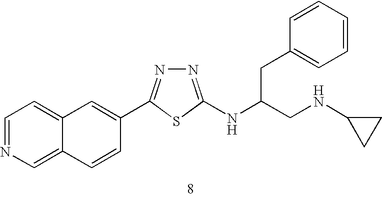

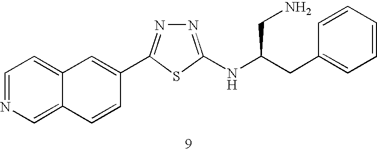

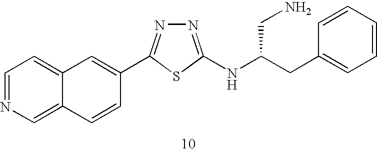

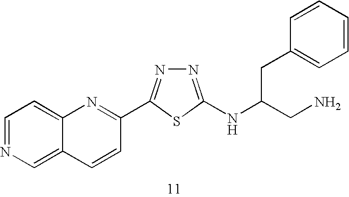

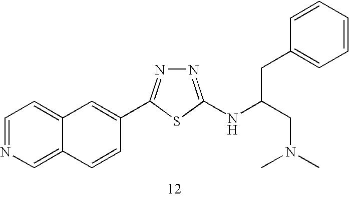

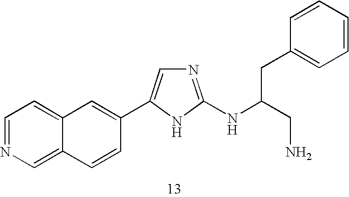

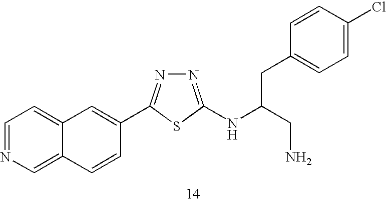

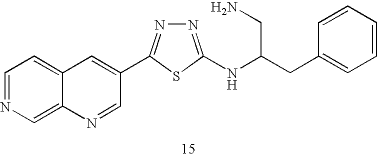

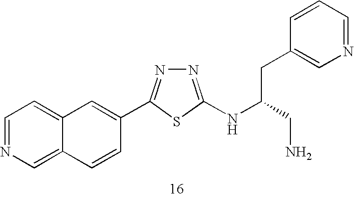

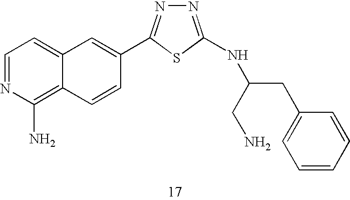

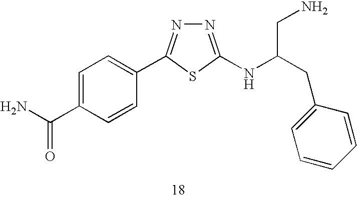

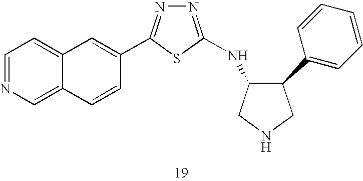

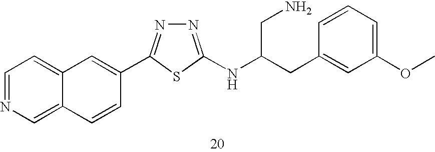

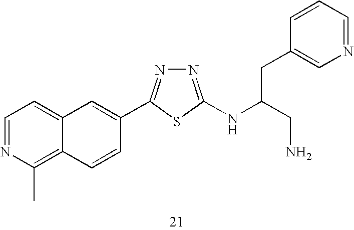

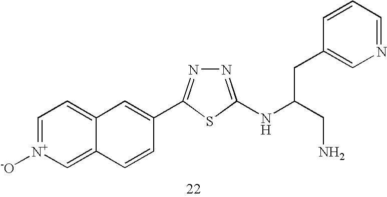

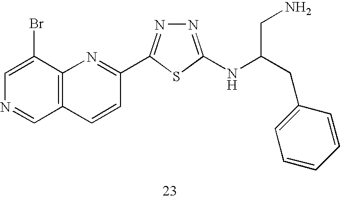

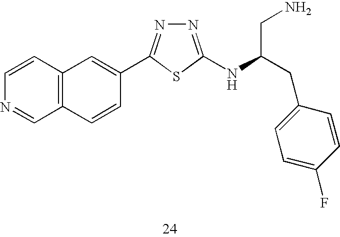

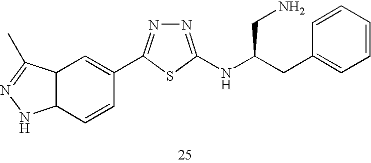

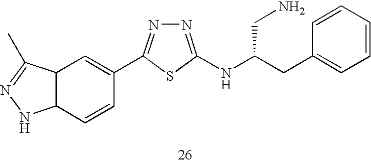

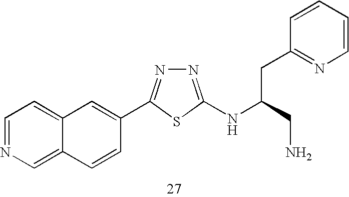

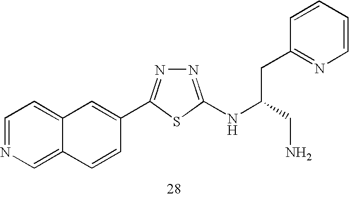

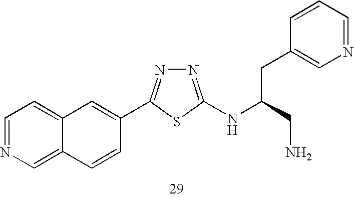

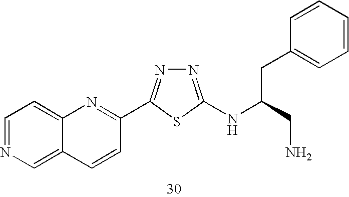

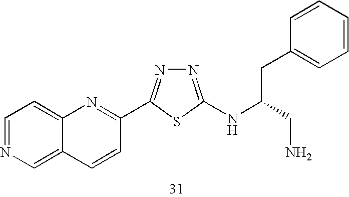

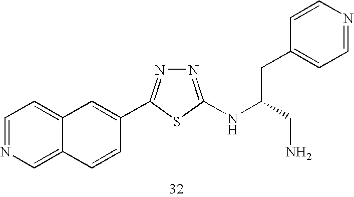

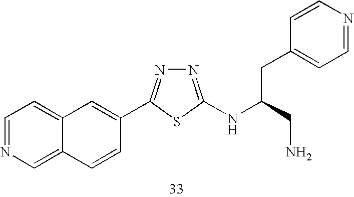

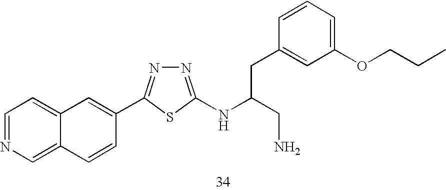

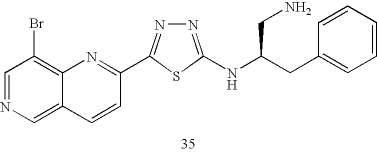

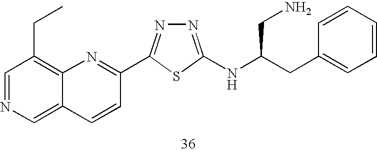

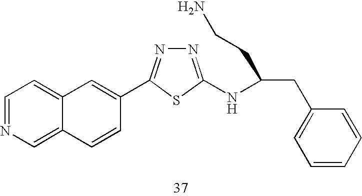

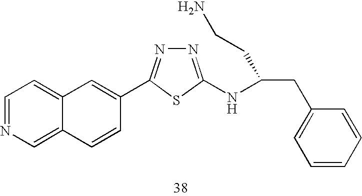

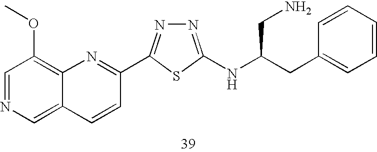

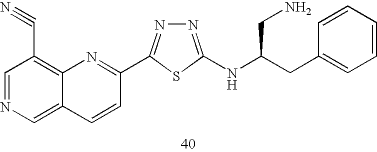

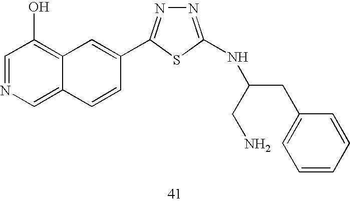

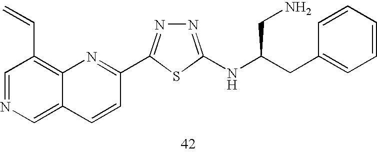

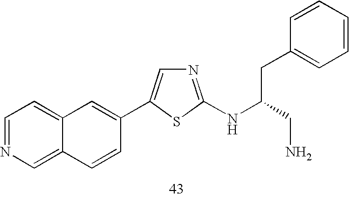

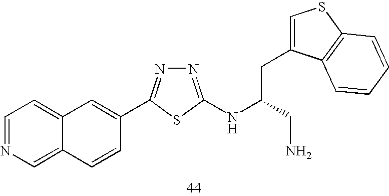

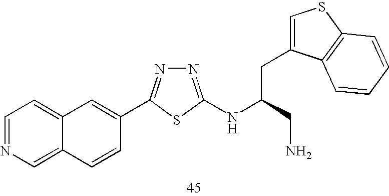

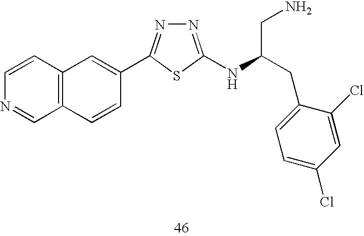

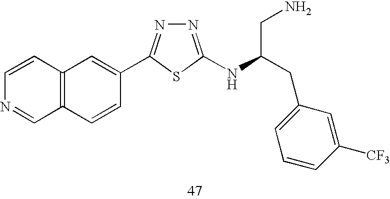

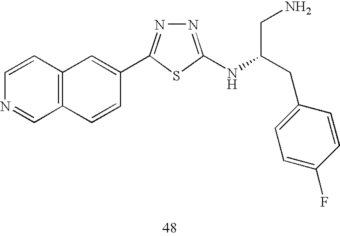

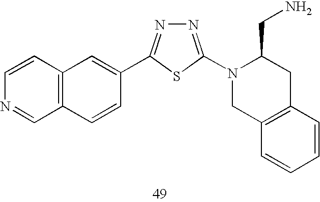

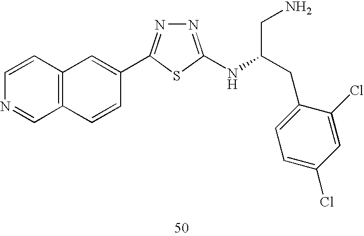

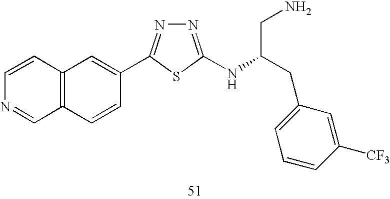

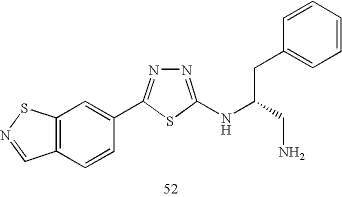

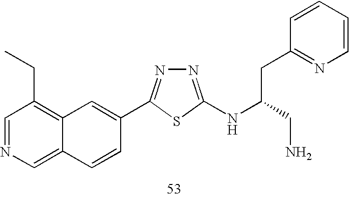

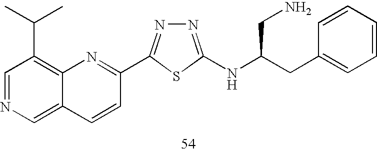

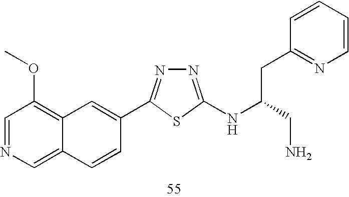

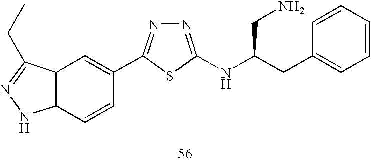

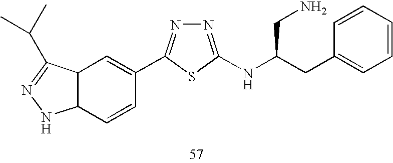

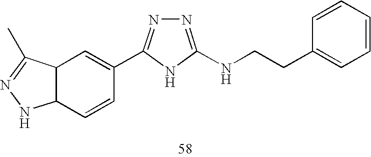

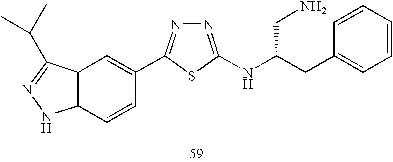

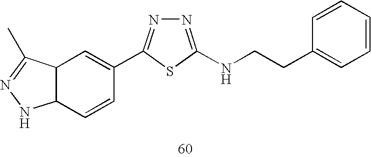

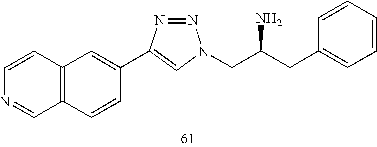

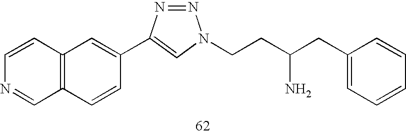

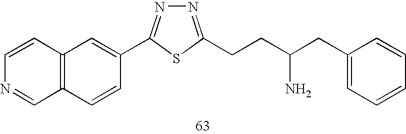

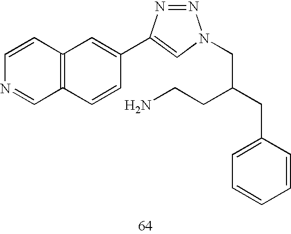

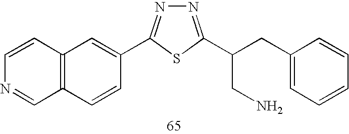

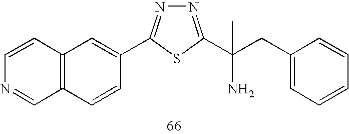

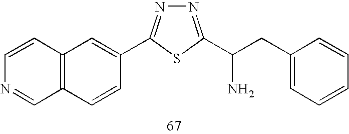

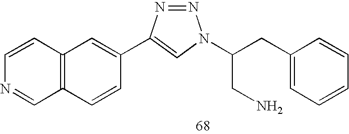

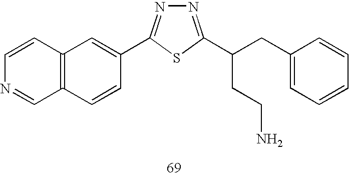

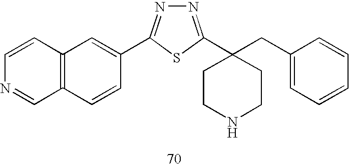

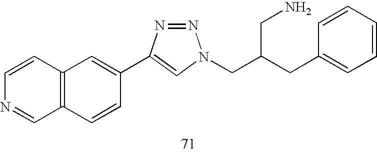

### 4.3 Methods for Making Heterocyclic Compounds

The Heterocyclic Compounds can be made by one skilled in the art using conventional organic syntheses and commercially available materials. By way of example and not limitation, a Heterocyclic Compound can be prepared as outlined in Schemes 1-8 shown below, as well as in the examples set forth in Section 5.1. It should be noted that one skilled in the art can modify the procedures set forth in the illustrative schemes and examples to arrive at the desired product.

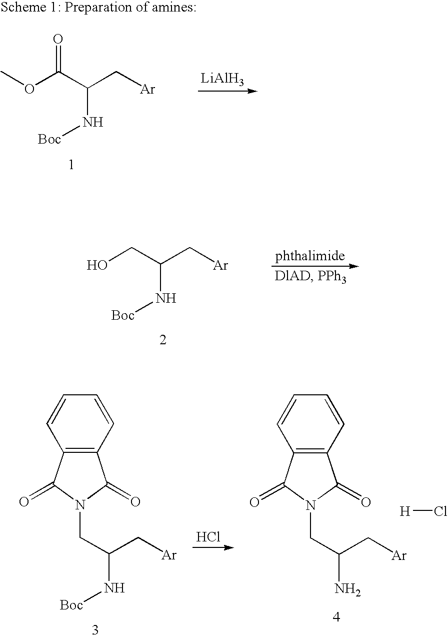

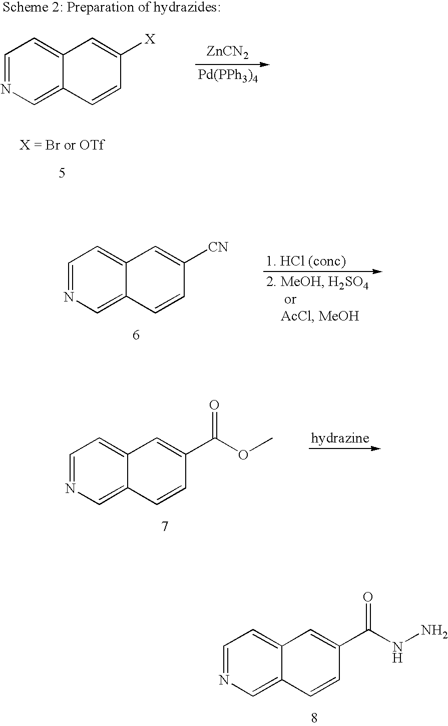

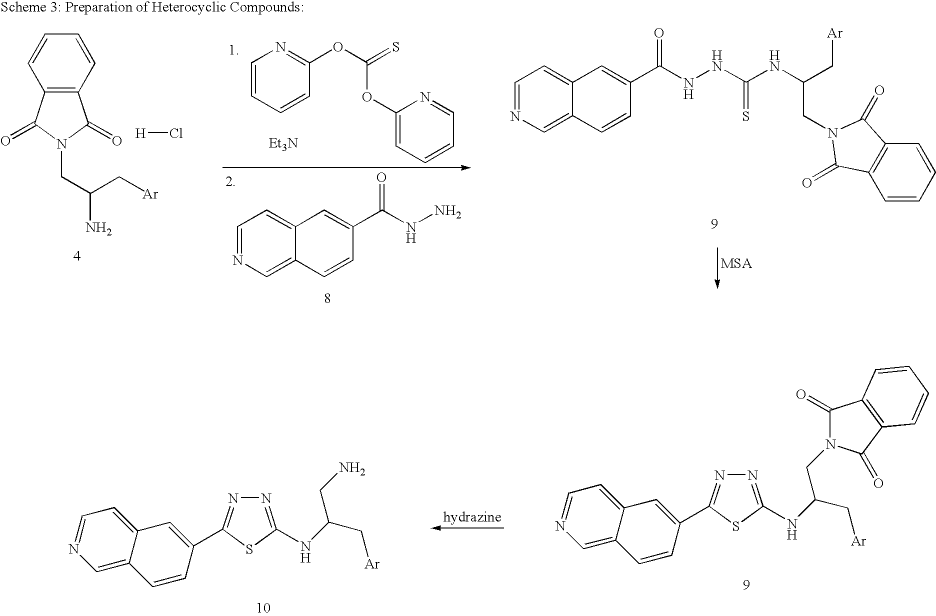

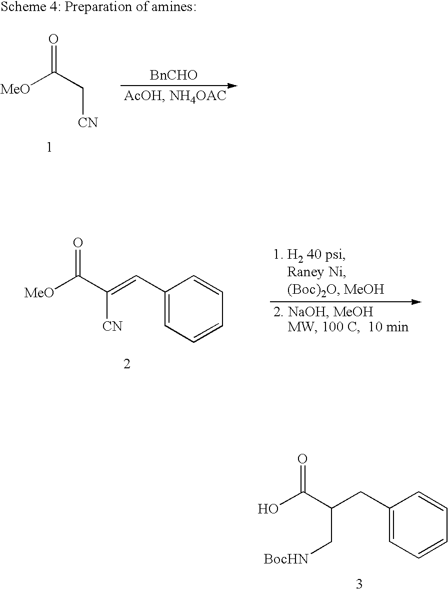

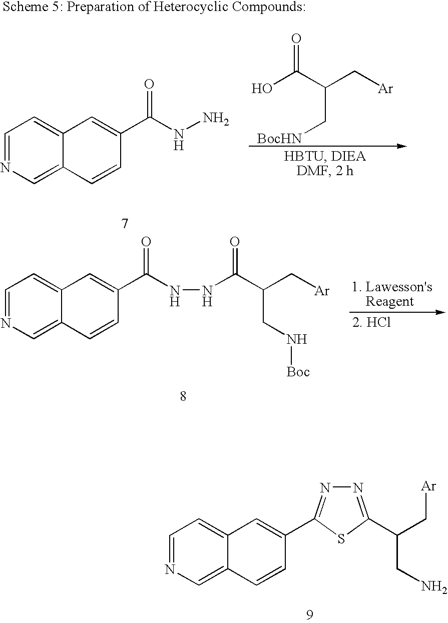

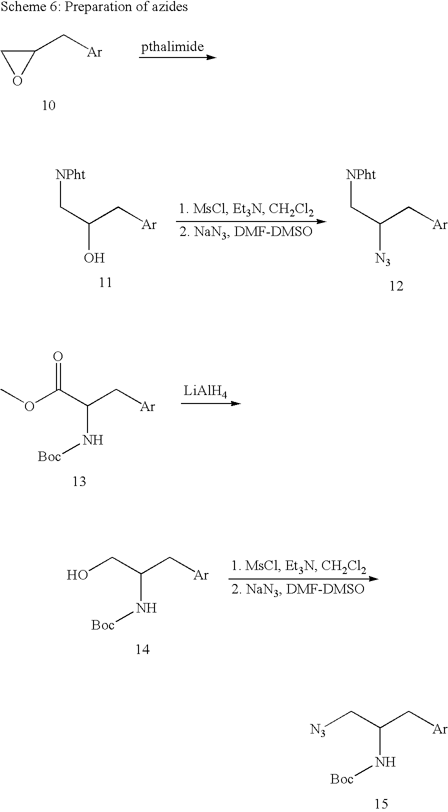

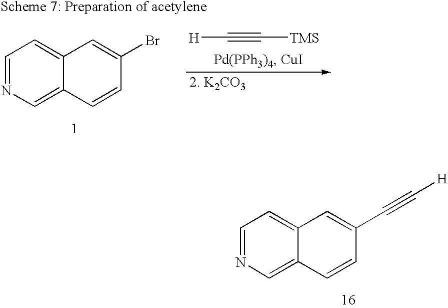

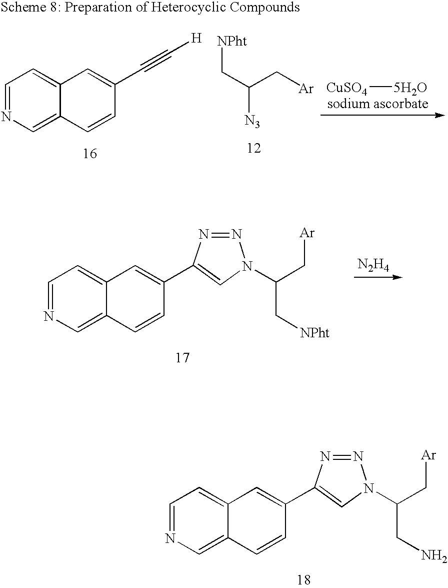

Pharmaceutically acceptable salts of the Heterocyclic Compounds can be formed by conventional and known techniques, such as by reacting a Heterocyclic Compound with a suitable acid as disclosed above. Such salts are typically formed in high yields at moderate temperatures, and often are prepared by merely isolating the compound from a suitable acidic wash in the final step of the synthesis. The salt-forming acid may dissolved in an appropriate organic solvent, or aqueous organic solvent, such as an alkanol, ketone or ester. On the other hand, if the Heterocyclic Compound is desired in the free base form, it may be isolated from a basic final wash step, according to known techniques. For example, a typical technique for preparing hydrochloride salt is to dissolve the free base in a suitable solvent, and dry the solution thoroughly, as over molecular sieves, before bubbling hydrogen chloride gas through it.

### 4.4 Methods of Use

Heterocyclic Compounds described herein have utility as pharmaceuticals to treat or prevent disease in animals or humans. Further, Heterocyclic Compounds described herein are active against protein kinases, including those involved in cancer, inflammatory conditions, immunological conditions and metabolic conditions. Without being limited by theory, it is thought the Heterocyclic Compounds are effective for treating and preventing cancer, inflammatory conditions, immunological conditions and metabolic conditions due to their ability to modulate (e.g., inhibit) kinases which are involved in the etiology of these conditions. Accordingly, provided herein are many uses of the Heterocyclic Compounds, including the treatment or prevention of those diseases set forth below. The methods provided herein comprise the administration of an effective amount of a Heterocyclic Compound to a patient in need thereof.

Representative immunological conditions that Heterocyclic Compounds are useful for treating or preventing include, but are not limited to, rheumatoid arthritis, rheumatoid spondylitis, osteoarthritis, multiple sclerosis, lupus, inflammatory bowel disease, ulcerative colitis, Crohn's disease, myasthenia gravis, Grave's disease and diabetes (e.g., Type I diabetes).

Representative inflammatory conditions that Heterocyclic Compounds are useful for treating or preventing include, but are not limited to, asthma and allergic rhinitis, bronchitis, chronic obstructive pulmonary disease, cystic fibrosis, inflammatory bowel disease, irritable bowel syndrome, Crohn's disease, mucous colitis, ulcerative colitis, diabetes (e.g., Type I diabetes and Type II diabetes) and obesity.

Representative metabolic conditions that Heterocyclic Compounds are useful for treating or preventing include, but are not limited to, obesity and diabetes (e.g., Type II diabetes).

In a particular embodiment, provided herein are methods for the treatment or prevention of insulin resistance. In certain embodiments, provided herein are methods for the treatment or prevention of insulin resistance that leads to diabetes (e.g., Type II diabetes).

In another embodiment, provided herein are methods for the treatment or prevention of syndrome X or metabolic syndrome.

In another embodiment, provide herein are methods for the treatment or prevention of diabetes.

In another embodiment, provide herein are methods for the treatment or prevention of Type II diabetes, Type I diabetes, slow-onset Type I diabetes, diabetes insipidus (e.g., neurogenic diabetes insipidus, nephrogenic diabetes insipidus, dipsogenic diabetes insipidus, or gestagenic diabetes insipidus), diabetes mellitus, gestational diabetes mellitus, polycystic ovarian syndrome, maturity-onset diabetes, juvenile diabetes, insulin-dependant diabetes, non-insulin dependant diabetes, malnutrition-related diabetes, ketosis-prone diabetes, pre-diabetes (e.g., impaired glucose metabolism), cystic fibrosis related diabetes, hemochromatosis and ketosis-resistant diabetes.

In another embodiment, provided herein are methods for the treatment or prevention of fibrotic diseases and disorders. In a particular embodiment, provided herein are methods for the treatment or prevention of idiopathic pulmonary fibrosis, myelofibrosis, hepatic fibrosis, steatofibrosis and steatohepatitis.

Representative cancers that Heterocyclic Compounds are useful for treating or preventing include, but are not limited to, cancers of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system. The cancer can be a solid tumor or a blood born tumor.

Cancers within the scope of the methods provided herein include those associated with BCR-ABL, and mutants or isoforms thereof, as well as kinases from the src kinase family, kinases from the Rsk kinase family, kinases from the CDK family, kinases from the MAPK kinase family, serine/threonine kinases (e.g., Akt) and tyrosine kinases such as Fes, Lyn, and Syk kinases, and mutants or isoforms thereof.

In a particular embodiment, provided herein are methods for the treatment or prevention of a disease or disorder associated with the modulation, for example inhibition, of a kinase, including, but are not limited to, tyrosine-protein kinase (SYK), tyrosine-protein kinase (ZAP-70), protein tyrosine kinase 2 beta (PYK2), focal adhesion kinase 1 (FAK), B lymphocyte kinase (BLK), hemopoietic cell kinase (HCK), v-yes-1 Yamaguchi sarcoma viral related oncogene homolog (LYN), T cell-specific protein-tyrosine kinase (LCK), proto-oncogene tyrosine-protein kinase (YES), proto-oncogene tyrosine-protein kinase (SRC), proto-oncogene tyrosine-protein kinase (FYN), proto-oncogene tyrosine-protein kinase (FGR), proto-oncogene tyrosine-protein kinase (FER), proto-oncogene tyrosine-protein kinase (FES), C-SRC kinase, protein-tyrosine kinase (CYL), tyrosine protein kinase (CSK), megakaryocyte-associated tyrosine-protein kinase (CTK), tyrosine-protein kinase receptor (EPH), Ephrin type-A receptor 1, Ephrin type-A receptor 4 (EPHA4), Ephrin type-B receptor 3 (EPHB3), Ephrin type-A receptor 8 (EPHA8), neurotrophic tyrosine kinase receptor, type 1 (NTRK1), protein-tyrosine kinase (PTK2), syk-related tyrosine kinase (SRK), protein tyrosine kinase (CTK), tyro3 protein tyrosine kinase (TYRO3), bruton agammaglobulinemia tyrosine kinase (BTK), leukocyte tyrosine kinase (LTK), protein-tyrosine kinase (SYK), protein-tyrosine kinase (STY), tek tyrosine kinase (TEK), elk-related tyrosine kinase (ERK), tyrosine kinase with immunoglobulin and egf factor homology domains (TIE), protein tyrosine kinase (TKF), neurotrophic tyrosine kinase, receptor, type 3 (NTRK3), mixed-lineage protein kinase-3 (MLK3), protein kinase, mitogen-activated 4 (PRKM4), protein kinase, mitogen-activated 1 (PRKM1), protein tyrosine kinase (PTK7), protein tyrosine kinase (EEK), minibrain (drosophila) homolog (MNBH), bone marrow kinase, x-linked (BMX), eph-like tyrosine kinase 1 (ETK1), macrophage stimulating 1 receptor (MST1R), btk-associated protein, 135 kd, lymphocyte-specific protein tyrosine kinase (LCK), fibroblast growth factor receptor-2 (FGFR2), protein tyrosine kinase-3 (TYK3), protein tyrosine kinase (TXK), tec protein tyrosine kinase (TEC), protein tyrosine kinase-2 (TYK2), eph-related receptor tyrosine kinase ligand 1 (EPLG1), t-cell tyrosine kinase (EMT), eph tyrosine kinase 1 (EPHT1), zona pellucida receptor tyrosine kinase, 95 kd (ZRK), protein kinase, mitogen-activated, kinase 1 (PRKMK1), eph tyrosine kinase 3 (EPHT3), growth arrest-specific gene-6 (GAS6), kinase insert domain receptor (KDR), axl receptor tyrosine kinase (AXL), fibroblast growth factor receptor-1 (FGFR1), v-erb-b2 avian erythroblastic leukemia viral oncogene homolog 2 (ERBB2), fms-like tyrosine kinase-3 (FLT3), neuroepithelial tyrosine kinase (NEP), neurotrophic tyrosine kinase receptor-related 3 (NTRKR3), eph-related receptor tyrosine kinase ligand 5 (EPLG5), neurotrophic tyrosine kinase, receptor, type 2 (NTRK2), receptor-like tyrosine kinase (RYK), tyrosine kinase, b-lymphocyte specific (BLK), eph tyrosine kinase 2 (EPHT2), eph-related receptor tyrosine kinase ligand 2 (EPLG2), glycogen storage disease VIII, eph-related receptor tyrosine kinase ligand 7 (EPLG7), janus kinase 1 (JAK1), fms-related tyrosine kinase-1 (FLT1), protein kinase, camp-dependent, regulatory, type I, alpha (PRKAR1A), wee-1 tyrosine kinase (WEE1), eph-like tyrosine kinase 2 (ETK2), receptor tyrosine kinase musk, insulin receptor (INSR), janus kinase 3 (JAK3), fms-related tyrosine kinase-3 ligand protein kinase c, beta 1 (PRKCB1), tyrosine kinase-type cell surface receptor (HER3), janus kinase 2 (JAK2), lim domain kinase 1 (LIMK1), dual specificity phosphatase 1 (DUSP1), hemopoietic cell kinase (HCK), tyrosine 3-monooxygenase/tryptophan 5-monooxygenase activation protein, eta polypeptide (YWHAH), ret proto-oncogene (RET), tyrosine 3-monooxygenase/tryptophan 5-monooxygenase activation protein, zeta polypeptide (YWHAZ), tyrosine 3-monooxygenase/tryptophan 5-monooxygenase activation protein, beta polypeptide (YWHAB), hepatoma transmembrane kinase (HTK), map kinase 6, phosphatidylinositol 3-kinase, catalytic, alpha polypeptide (PIK3CA), cyclin-dependent kinase inhibitor 3 (CDKN3), diacylglycerol kinase, delta, 130 kd, protein-tyrosine phosphatase, nonreceptor type, 13 (PTPN13), abelson murine leukemia viral oncogene homolog 1 (ABL1), diacylglycerol kinase, alpha (DAGK1), focal adhesion kinase 2, epithelial discoidin domain receptor 1 (EDDR1), anaplastic lymphoma kinase (ALK), phosphatidylinositol 3-kinase, catalytic, gamma polypeptide (PIK3CG), phosphatidylinositol 3-kinase regulatory subunit, (PIK3R1), eph homology kinase-1 (EHK1), v-kit hardy-zuckerman 4 feline sarcoma viral oncogene homolog (KIT), fibroblast growth factor receptor-3 (FGFR3), vascular endothelial growth factor c (VEGFC), epidermal growth factor receptor (EGFR), oncogene (TRK), growth factor receptor-bound protein-7 (GRB7), ras p21 protein activator (RASA2), met proto-oncogene (MET), src-like adapter (SLA), vascular endothelial growth factor (VEGF), vascular endothelial growth factor receptor (VEGFR), nerve growth factor receptor (NGFR), platelet derived growth factor receptor (PDGFR), platelet derived growth factor receptor beta (PDGFRB), dual-specificity tyrosine-(Y)-phosphorylation regulated kinase 2 (DYRK2), dual-specificity tyrosine-(Y)-phosphorylation regulated kinase 3 (DYRK3), dual-specificity tyrosine-(Y)-phosphorylation regulated kinase 4 (DYRK4), dual-specificity tyrosine-(Y)-phosphorylation regulated kinase 1A (DYRK1A), dual-specificity tyrosine-(Y)-phosphorylation regulated kinase 1B (DYRK1B), CDC-like kinase 1 (CLK1), protein tyrosine kinase STY, CDC-like kinase 4 (CLK4), CDC-like kinase 2 (CLK2) or CDC-like kinase 3 (CLK3).

In another embodiment, provided herein are methods for the treatment or prevention of a disease or disorder associated with the modulation, for example inhibition, of serine/threonine kinases or related molecules, including, but not limited to, Akt/protein kinase B, protein kinase A (PKA), CK2, cyclin-dependent kinase 7 (CDK7), rac serine/threonine protein kinase, serine-threonine protein kinase n (PKN), serine/threonine protein kinase 2 (STK2), zipper protein kinase (ZPK), protein-tyrosine kinase (STY), bruton agammaglobulinemia tyrosine kinase (BTK), mkn28 kinase, protein kinase, x-linked (PRKX), elk-related tyrosine kinase (ERK), ribosomal protein s6 kinase, 90 kd, polypeptide 3 (RPS6KA3), glycogen storage disease VIII, death-associated protein kinase 1 (DAPK1), pctaire protein kinase 1 (PCTK1), protein kinase, interferon-inducible double-stranded rna (PRKR), activin a receptor, type II-like kinase 1 (ACVRLK1), protein kinase, camp-dependent, catalytic, alpha (PRKACA), protein kinase, y-linked (PRKY), G protein-coupled receptor kinase 2 (GPRK21), protein kinase c, theta form (PRKCQ), lim domain kinase 1 (LIMK1), phosphoglycerate kinase 1 PGK1), lim domain kinase 2 (LIMK2), c-jun kinase, activin a receptor, type II-like kinase 2 (ACVRLK2), janus kinase 1 (JAK1), elk1 motif kinase (EMK1), male germ cell-associated kinase (MAK), casein kinase 2, alpha-prime subunit (CSNK2A2), casein kinase 2, beta polypeptide (CSNK2B), casein kinase 2, alpha 1 polypeptide (CSNK2A1), ret proto-oncogene (RET), hematopoietic progenitor kinase 1, conserved helix-loop-helix ubiquitous kinase (CHUK), casein kinase 1, delta (CSNK1D), casein kinase 1, epsilon (CSNK1E), v-akt murine thymoma viral oncogene homolog 1 (AKT1), tumor protein p53 (TP53), protein phosphatase 1, regulatory (inhibitor) subunit 2 (PPP1R2), oncogene pim-1 (PIM1), transforming growth factor-beta receptor, type II (TGFBR2), transforming growth factor-beta receptor, type I (TGFBR1), v-raf murine sarcoma viral oncogene homolog b1 (BRAF), bone morphogenetic receptor type II (BMPR2), v-raf murine sarcoma 3611 viral oncogene homolog 1 (ARAF1), v-raf murine sarcoma 3611 viral oncogene homolog 2 (ARAF2), protein kinase C (PKC), v-kit hardy-zuckerman 4 feline sarcoma viral oncogene homolog (KIT) or c-KIT receptor (KITR).

In another embodiment, provided herein are methods for the treatment or prevention of a disease or disorder associated with the modulation, for example inhibition, of a MAP kinase, including, but not limited to, mitogen-activated protein kinase 3 (MAPK3), p44erk1, p44mapk, mitogen-activated protein kinase 3 (MAP kinase 3; p44), ERK1, PRKM3, P44ERK1, P44MAPK, mitogen-activated protein kinase 1 (MAPK1), mitogen-activated protein kinase kinase 1 (MEK1), MAP2K1protein tyrosine kinase ERK2, mitogen-activated protein kinase 2, extracellular signal-regulated kinase 2, protein tyrosine kinase ERK2, mitogen-activated protein kinase 2, extracellular signal-regulated kinase 2, ERK, p38, p40, p41, ERK2, ERT1, MAPK2, PRKM1, PRKM2, P42MAPK, p41mapk, mitogen-activated protein kinase 7 (MAPK7), BMK1 kinase, extracellular-signal-regulated kinase 5, BMK1, ERK4, ERK5, PRKM7, nemo-like kinase (NLK), likely ortholog of mouse nemo like kinase, mitogen-activated protein kinase 8 (MAPK8), protein kinase JNK1, JNK1 beta protein kinase, JNK1 alpha protein kinase, c-Jun N-terminal kinase 1, stress-activated protein kinase JNK1, JNK, JNK1, PRKM8, SAPK1, JNK1A2, JNK21B1/2, mitogen-activated protein kinase 10 (MAPK10), c-Jun kinase 3, JNK3 alpha protein kinase, c-Jun N-terminal kinase 3, stress activated protein kinase JNK3, stress activated protein kinase beta, mitogen-activated protein kinase 9 (MAPK9), MAP kinase 9, c-Jun kinase 2, c-Jun N-terminal kinase 2, stress-activated protein kinase JNK2, JNK2, JNK2A, JNK2B, PRKM9, JNK-55, JNK2BETA, p54aSAPK, JNK2ALPHA, mitogen-activated protein kinase 14 (MAPK14), p38 MAP kinase, MAP kinase Mxi2, Csaids binding protein, MAX-interacting protein 2, stress-activated protein kinase 2A, p38 mitogen activated protein kinase, cytokine suppressive anti-inflammatory drug binding protein, RK, p38, EXIP, Mxi2, CSBP1, CSBP2, CSPB1, PRKM14, PRKM15, SAPK2A, p38ALPHA, mitogen-activated protein kinase 11 (MAPK13), stress-activated protein kinase-2, stress-activated protein kinase-2b, mitogen-activated protein kinase p38-2, mitogen-activated protein kinase p38beta, P38B, SAPK2, p38-2, PRKM11, SAPK2B, p38Beta, P38BETA2, mitogen-activated protein kinase 13 (MAPK13), stress-activated protein kinase 4, mitogen-activated protein kinase p38 delta, SAPK4, PRKM13, p38delta, mitogen-activated protein kinase 12 (MAPK12), p38gamma, stress-activated protein kinase 3, mitogen-activated protein kinase 3, ERK3, ERK6, SAPK3, PRKM12, SAPK-3, P38GAMMA, mitogen-activated protein kinase 6 (MAPK6), MAP kinase isoform p97, mitogen-activated 5 protein kinase, mitogen-activated 6 protein kinase, extracellular signal-regulated kinase 3, extracellular signal-regulated kinase, p97, ERK3, PRKM6, p97MAPK, mitogen-activated protein kinase 4 (MAPK4), Erk3-related protein kinase, mitogen-activated 4 protein kinase (MAP kinase 4; p63), PRKM4, p63MAPK, ERK3-RELATED or Extracellular signal-regulated kinase 8 (ERK7).

More particularly, cancers and related disorders that can be treated or prevented by methods and compositions provided herein include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome (or a symptom thereof such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia and chronic myelomonocytic leukemia (CMML), chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions provided herein are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, glioblastoma multiforme, neuroblastoma, glioma, and schwannomas; solid and blood born tumors; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions disclosed herein. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, or uterus. In other specific embodiments, sarcoma, melanoma, or leukemia is treated or prevented.

In another embodiment, the methods and compositions provided herein are also useful for administration to patients in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiencies, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy.

In another embodiment, provided herein are methods for the treatment of myeloproliferative disorders or myelodysplastic syndromes, comprising administering to a patient in need thereof an effective amount of a Heterocyclic Compound or a composition thereof. In certain embodiments, the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

In another embodiment, provided herein are methods for the treatment of cancer or tumors resistant to other kinase inhibitors such as imatinib mesylate (STI-571 or Gleevec™) treatment, comprising administering to a patient in need thereof an effective amount of a Heterocyclic Compound or a composition thereof. In a particular embodiment, provided herein are methods for the treatment of leukemias, including, but not limited to, gastrointestinal stromal tumor (GIST), acute lymphocytic leukemia or chronic myelocytic leukemia resistant to imatinib mesylate (STI-571 or Gleevec™) treatment, comprising administering to a patient in need thereof an effective amount of a Heterocyclic Compound or a composition thereof.

Specific cancers include, but are not limited to, leukemias such as chronic lymphocytic leukemia, chronic myelocytic leukemia, acute lymphoblastic leukemia, acute myelogenous leukemia, and acute myeloblastic leukemia; advanced malignancy, amyloidosis, neuroblastoma, meningioma, hemangiopericytoma, multiple brain metastases, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In one embodiment, the cancer is primary or metastatic. In another embodiment, the cancer is relapsed, refractory or resistance to chemotherapy or radiation.

In another embodiment, provided herein are methods for treating, preventing or managing a lymphoma comprising administering an effective amount of a Heterocyclic Compound to a patient in need thereof. Specific lymphomas include, but are not limited to, mantle cell lymphoma, MCL, lymphocytic lymphoma of intermediate differentiation, intermediate lymphocytic lymphoma, ILL, diffuse poorly differentiated lymphocytic lymphoma, PDL, centrocytic lymphoma, diffuse small-cleaved cell lymphoma, DSCCL, follicular lymphoma, and any type of the mantle cell lymphoma that can be seen under the microscope (nodular, diffuse, blastic and mentle zone lymphoma). The term “lymphoma” refers a heterogenous group of neoplasms arising in the reticuloendothelial and lymphatic systems. Non-Hodgkin's lymphoma (NHL) refers to malignant monoclonal proliferation of lymphoid cells in sites of the immune system, including lymph nodes, bone marrow, spleen, liver and gastrointestinal tract. The NHL includes, but is not limited to, mantle cell lymphoma, MCL, lymphocytic lymphoma of intermediate differentiation, intermediate lymphocytic lymphoma, ILL, diffuse poorly differentiated lymphocytic lymphoma, PDL, centrocytic lymphoma, diffuse small-cleaved cell lymphoma, DSCCL, follicular lymphoma, and any type of the mantle cell lymphomas that can be seen under the microscope (nodular, diffuse, blastic and mentle zone lymphoma).

In one embodiment, provided herein are methods for treating patients who have been previously treated for cancer, but are non-responsive to standard therapies, as well as those who have not previously been treated. Also provided herein are methods for treating patients regardless of patient's age, although some diseases or disorders are more common in certain age groups. Further provided herein are methods for treating patients who have undergone surgery in an attempt to treat cancer, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In one embodiment, provided herein are methods for treating or preventing a disease or disorder treatable or preventable by modulating a kinase pathway, in one embodiment, the Akt pathway, comprising administering an effective amount of a Heterocyclic Compound to a patient in need of the treating or preventing. Particular diseases which are treatable or preventable by modulating, for example, inhibiting, a kinase pathway, in one embodiment, the Akt pathway, include, but are not limited to, rheumatoid arthritis; rheumatoid spondylitis; osteoarthritis; gout; asthma, bronchitis; allergic rhinitis; chronic obstructive pulmonary disease; cystic fibrosis; inflammatory bowel disease; irritable bowel syndrome; mucous colitis; ulcerative colitis; Crohn's disease; Huntington's disease; gastritis; esophagitis; hepatitis; pancreatitis; nephritis; multiple sclerosis; lupus erythematosus; Type II diabetes; obesity; atherosclerosis; restenosis following angioplasty; left ventricular hypertrophy; myocardial infarction; stroke; ischemic damages of heart, lung, gut, kidney, liver, pancreas, spleen and brain; acute or chronic organ transplant rejection; preservation of the organ for transplantation; organ failure or loss of limb (e.g., including, but not limited to, that resulting from ischemia-reperfusion injury, trauma, gross bodily injury, car accident, crush injury or transplant failure); graft versus host disease; endotoxin shock; multiple organ failure; psoriasis; burn from exposure to fire, chemicals or radiation; eczema; dermatitis; skin graft; ischemia; ischemic conditions associated with surgery or traumatic injury (e.g., vehicle accident, gunshot wound or limb crush); epilepsy; Alzheimer's disease; Parkinson's disease; immunological response to bacterial or viral infection; cachexia; angiogenic and proliferative dieseases; solid tumor; and cancers of a variety of tissues such as colon, rectum, prostate, liver, lung, bronchus, pancreas, brain, head, neck, stomach, skin, kidney, cervix, blood, larynx, esophagus, mouth, pharynx, urinary bladder, ovary or uterine.

A Heterocyclic Compound can be combined with other pharmacologically active compounds (“second active agents”) in methods and compositions described herein. It is believed that certain combinations may work synergistically in the treatment of particular types diseases or disorders, and conditions and symptoms associated with such diseases or disorders. A Heterocyclic Compound can also work to alleviate adverse effects associated with certain second active agents, and vice versa.

One or more second active ingredients or agents can be used in the methods and compositions described herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Specific examples of the active agents are anti-CD40 monoclonal antibodies (such as, for example, SGN-40); histone deacetylyase inhibitors (such as, for example, SAHA and LAQ 824); heat-shock protein-90 inhibitors (such as, for example, 17-AAG); insulin-like growth factor-1 receptor kinase inhibitors; vascular endothelial growth factor receptor kinase inhibitors (such as, for example, PTK787); insulin growth factor receptor inhibitors; lysophosphatidic acid acyltransrerase inhibitors; IkB kinase inhibitors; p38MAPK inhibitors; EGFR inhibitors (such as, for example, gefitinib and erlotinib HCL); HER-2 antibodies (such as, for example, trastuzumab (Herceptin®) and pertuzumab (Omnitarg™)); VEGFR antibodies (such as, for example, bevacizumab (Avastin™)); VEGFR inhibitors (such as, for example, flk-1 specific kinase inhibitors, SU5416 and ptk787/zk222584); P13K inhibitors (such as, for example, wortmannin); C-Met inhibitors (such as, for example, PHA-665752); monoclonal antibodies (such as, for example, rituximab (Rituxan®), tositumomab (Bexxar®), edrecolomab (Panorex®) and G250); and anti-TNF-α antibodies. Examples of small molecule active agents include, but are not limited to, small molecule anti-cancer agents and antibiotics (e.g., clarithromycin).

Specific second active compounds that can be combined with a Heterocyclic Compound vary depending on the specific indication to be treated, prevented or managed.

For instance, for the treatment, prevention or management of cancer, second active agents include, but are not limited to: semaxanib; cyclosporin; etanercept; doxycycline; bortezomib; acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other second agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clathromycin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, 2-methoxyestradiol, telomestatin, inducers of apoptosis in multiple myeloma cells (such as, for example, TRAIL), bortezomib, statins, semaxanib, cyclosporin, etanercept, doxycycline, bortezomib, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-1, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

Similarly, examples of specific second agents according to the indications to be treated, prevented, or managed can be found in the following references, all of which are incorporated herein in their entireties: U.S. Pat. Nos. 6,281,230 and 5,635,517; U.S. application Ser. Nos. 10/411,649, 10/483,213, 10/411,656, 10/693,794, 10/699,154, and 10/981,189; and U.S. provisional application Nos. 60/554,923, 60/565,172, 60/626,975, 60/630,599, 60/631,870, and 60/533,862.

Examples of additional second active agents include, but are not limited to, conventional therapeutics used to treat or prevent pain such as antidepressants, anticonvulsants, antihypertensives, anxiolytics, calcium channel blockers, muscle relaxants, non-narcotic analgesics, opioid analgesics, anti-inflammatories, cox-2 inhibitors, immunomodulatory agents, alpha-adrenergic receptor agonists or antagonists, immunosuppressive agents, corticosteroids, hyperbaric oxygen, ketamine, other anesthetic agents, NMDA antagonists, and other therapeutics found, for example, in the Physician's Desk Reference 2003. Specific examples include, but are not limited to, salicylic acid acetate (Aspirin®), celecoxib (Celebrex®), Enbrel®, ketamine, gabapentin (Neurontin®), phenyloin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®), mexiletine, nifedipine, propranolol, tramadol, lamotrigine, ziconotide, ketamine, dextromethorphan, benzodiazepines, baclofen, tizanidine and phenoxybenzamine.

Examples of additional second active agents include, but are not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound or an antiangiogenesis compound, or a combination thereof. Specific examples include, but are not limited to, verteporfin, purlytin, an angiostatic steroid, rhuFab, interferon-2ÿ, pentoxifylline, tin etiopurpurin, motexafin lutetium, 9-fluoro-11,21-dihydroxy-16,17-1-methylethylidinebis(oxy)pregna-1,4-diene-3,20-dione, latanoprost (see U.S. Pat. No. 6,225,348), tetracycline and its derivatives, rifamycin and its derivatives, macrolides, metronidazole (U.S. Pat. Nos. 6,218,369 and 6,015,803), genistein, genistin, 6′-O-Mal genistin, 6′-O-Ac genistin, daidzein, daidzin, 6′-O-Mal daidzin, 6′-O-Ac daidzin, glycitein, glycitin, 6′-O-Mal glycitin, biochanin A, formononetin (U.S. Pat. No. 6,001,368), triamcinolone acetomide, dexamethasone (U.S. Pat. No. 5,770,589), thalidomide, glutathione (U.S. Pat. No. 5,632,984), basic fibroblast growth factor (bFGF), transforming growth factor b (TGF-b), brain-derived neurotrophic factor (BDNF), plasminogen activator factor type 2 (PAI-2), EYE101 (Eyetech Pharmaceuticals), LY333531 (Eli Lilly), Miravant, and RETISERT implant (Bausch & Lomb). All of the references cited above are incorporated herein in their entireties by reference.

Examples of additional second active agents include, but are not limited to, keratolytics, retinoids, α-hydroxy acids, antibiotics, collagen, botulinum toxin, interferon, and immunomodulatory agents. Specific examples include, but are not limited to, 5-fluorouracil, masoprocol, trichloroacetic acid, salicylic acid, lactic acid, ammonium lactate, urea, tretinoin, isotretinoin, antibiotics, collagen, botulinum toxin, interferon, corticosteroid, transretinoic acid and collagens such as human placental collagen, animal placental collagen, Dermalogen, AlloDerm, Fascia, Cymetra, Autologen, Zyderm, Zyplast, Resoplast, and Isolagen.

Examples of additional second active agents include, but are not limited to, anticoagulants, diuretics, cardiac glycosides, calcium channel blockers, vasodilators, prostacyclin analogues, endothelin antagonists, phosphodiesterase inhibitors (e.g., PDE V inhibitors), endopeptidase inhibitors, lipid lowering agents, thromboxane inhibitors, and other therapeutics known to reduce pulmonary artery pressure. Specific examples include, but are not limited to, warfarin (Coumadin®), a diuretic, a cardiac glycoside, digoxin-oxygen, diltiazem, nifedipine, a vasodilator such as prostacyclin (e.g., prostaglandin I2 (PGI2), epoprostenol (EPO, Floran®), treprostinil (Remodulin®), nitric oxide (NO), bosentan (Tracleer®), amlodipine, epoprostenol (Floran®), treprostinil (Remodulin®), prostacyclin, tadalafil (Clalis®), simvastatin (Zocor®), omapatrilat (Vanlev®), irbesartan (Avapro®), pravastatin (Pravachol®), digoxin, L-arginine, iloprost, betaprost, and sildenafil (Viagra®).

Examples of additional second active agents include, but are not limited to, anthracycline, platinum, alkylating agent, oblimersen (Genasense®), cisplatinum, cyclophosphamide, temodar, carboplatin, procarbazine, gliadel, tamoxifen, topotecan, methotrexate, taxotere, irinotecan, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, bleomycin, hyaluronidase, mitomycin C, mepacrine, thiotepa, tetracycline and gemcitabine.

Examples of additional second active agents include, but are not limited to, chloroquine, quinine, quinidine, pyrimethamine, sulfadiazine, doxycycline, clindamycin, mefloquine, halofantrine, primaquine, hydroxychloroquine, proguanil, atovaquone, azithromycin, suramin, pentamidine, melarsoprol, nifurtimox, benznidazole, amphotericin B, pentavalent antimony compounds (e.g., sodium stiboglucuronate), interfereon gamma, itraconazole, a combination of dead promastigotes and BCG, leucovorin, corticosteroids, sulfonamide, spiramycin, IgG (serology), trimethoprim, and sulfamethoxazole.

Examples of additional second active agents include, but are not limited to: antibiotics (therapeutic or prophylactic) such as, but not limited to, ampicillin, clarithromycin, tetracycline, penicillin, cephalosporins, streptomycin, kanamycin, and erythromycin; antivirals such as, but not limited to, amantadine, rimantadine, acyclovir, and ribavirin; immunoglobulin; plasma; immunologic enhancing drugs such as, but not limited to, levami sole and isoprinosine; biologics such as, but not limited to, gammaglobulin, transfer factor, interleukins, and interferons; hormones such as, but not limited to, thymic; and other immunologic agents such as, but not limited to, B cell stimulators (e.g., BAFF/BlyS), cytokines (e.g., IL-2, IL-4, and IL-5), growth factors (e.g., TGF-ÿ), antibodies (e.g., anti-CD40 and IgM), oligonucleotides containing unmethylated CpG motifs, and vaccines (e.g., viral and tumor peptide vaccines).

Examples of additional second active agents include, but are not limited to: a dopamine agonist or antagonist, such as, but not limited to, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, and Symmetrel; a MAO inhibitor, such as, but not limited to, iproniazid, clorgyline, phenelzine and isocarboxazid; a COMT inhibitor, such as, but not limited to, tolcapone and entacapone; a cholinesterase inhibitor, such as, but not limited to, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, and demecarium; an anti-inflammatory agent, such as, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids; and an antiemetic agent, such as, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and a mixture thereof.

Examples of additional second active agents include, but are not limited to, immunomodulatory agents, immunosuppressive agents, antihypertensives, anticonvulsants, fibrinolytic agents, antiplatelet agents, antipsychotics, antidepressants, benzodiazepines, buspirone, amantadine, and other known or conventional agents used in patients with CNS injury/damage and related syndromes. Specific examples include, but are not limited to: steroids (e.g., glucocorticoids, such as, but not limited to, methylprednisolone, dexamethasone and betamethasone); an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone; a cAMP analog including, but not limited to, db-cAMP; an agent comprising a methylphenidate drug, which comprises 1-threo-methylphenidate, d-threo-methylphenidate, dl-threo-methylphenidate, 1-erythro-methylphenidate, d-erythro-methylphenidate, dl-erythro-methylphenidate, and a mixture thereof; and a diuretic agent such as, but not limited to, mannitol, furosemide, glycerol, and urea.

Examples of additional second active agents include, but are not limited to, a tricyclic antidepressant agent, a selective serotonin reuptake inhibitor, an antiepileptic agent (gabapentin, pregabalin, carbamazepine, oxcarbazepine, levitiracetam, topiramate), an antiaryhthmic agent, a sodium channel blocking agent, a selective inflammatory mediator inhibitor, an opioid agent, a second immunomodulatory compound, a combination agent, and other known or conventional agents used in sleep therapy. Specific examples include, but are not limited to, Neurontin, oxycontin, morphine, topiramate, amitryptiline, nortryptiline, carbamazepine, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, Symmetrel, iproniazid, clorgyline, phenelzine, isocarboxazid, tolcapone, entacapone, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, demecarium, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone, benzbromarone, betamethasone and other glucocorticoids, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and a mixture thereof.

Examples of additional second active agents include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; and G-CSF; hydroxyurea; butyrates or butyrate derivatives; nitrous oxide; HEMOXIN™ (NIPRISAN™; see U.S. Pat. No. 5,800,819); Gardos channel antagonists such as clotrimazole and triaryl methane derivatives; Deferoxamine; protein C; and transfusions of blood, or of a blood substitute such as Hemospan™ or Hemospan™ PS (Sangart).

Administration of a Heterocyclic Compound and a second active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for Heterocyclic Compounds is oral. Preferred routes of administration for the second active agents or ingredients of the invention are known to those of ordinary skill in the art. See, e.g., Physicians' Desk Reference, 1755-1760 (56th ed., 2002).

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of a Heterocyclic Compound and any optional additional active agents concurrently administered to the patient.

Further provided herein are methods of reducing, treating and/or preventing adverse or undesired effects associated with conventional therapy including, but not limited to, surgery, chemotherapy, radiation therapy, hormonal therapy, biological therapy and immunotherapy. Heterocyclic Compounds and other active ingredients can be administered to a patient prior to, during, or after the occurrence of the adverse effect associated with conventional therapy.

### 4.5 Pharmaceutical Compositions and Routes of Administration

The Heterocyclic Compounds can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the Heterocyclic Compound in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a Heterocyclic Compound to be administered to a patient is rather widely variable and can be subject to the judgment of a health-care practitioner. In general, the Heterocyclic Compounds can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the Heterocyclic Compound administered will depend on such factors as the solubility of the active component, the formulation used and the route of administration.

In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Heterocyclic Compound to a patient in need thereof.

In another embodiment, provided herein are methods for the treatment or prevention of a disease or disorder comprising the administration of about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a Heterocyclic Compound to a patient in need thereof. In a particular embodiment, the methods disclosed herein comprise the administration of 400 mg/day, 600 mg/day or 800 mg/day of a Heterocyclic Compound to a patient in need thereof.

In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of a Heterocyclic Compound.

In a particular embodiment, provided herein are unit dosage formulation comprising about 100 mg or 400 mg of a Heterocyclic Compound.

In another embodiment, provided herein are unit dosage formulations that comprise 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a Heterocyclic Compound.

A Heterocyclic Compound can be administered once, twice, three, four or more times daily. In a particular embodiment, doses of 600 mg or less are administered as a a once daily dose and doses of more than 600 mg are administered twice daily in an amount equal to one half of the total daily dose.

A Heterocyclic Compound can be administered orally for reasons of convenience. In one embodiment, when administered orally, a Heterocyclic Compound is administered with a meal and water. In another embodiment, the Heterocyclic Compound is dispersed in water or juice (e.g., apple juice or orange juice) and administered orally as a suspension.

The Heterocyclic Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a Heterocyclic Compound without an additional carrier, excipient or vehicle.

In another embodiment, provided herein are compositions comprising an effective amount of a Heterocyclic Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Heterocyclic Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Heterocyclic Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Heterocyclic Compound can be delayed or prolonged by proper formulation. For example, a slowly soluble pellet of the Heterocyclic Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Heterocyclic Compound in oily or emulsified vehicles that allow it to disperse slowly in the serum.

### 5. EXAMPLES

The following Examples are presented by way of illustration, not limitation.

### 5.1 Synthetic Examples

General Synthetic Procedures

General Procedure A (Phthalamide Deprotection):

Hydrazine is added to phthalimide substrate in ethanol and the resulting mixture is heated on a 60° C. oil bath for about 2-18 hours. After cooling to room temperature, the solution is diluted with EtOAc and the resulting white solid by-product is removed by filtration. The filtrate is concentrated under reduced pressure to provide crude product.

General Procedure B (Cyclization):

Substrate is dissolved in methane sulfonic acid and stirred at either room temperature or about 60° C. After stirring about 2-24 hours, the reaction is diluted with water. The resulting acidic solution is neutralized with either ammonium hydroxide or saturated potassium bicarbonate and washed with CH₂Cl₂. Organic fractions are combined and washed with water and saturated sodium chloride, dried over magnesium sulfate, filtered, and solvent is removed under reduced pressure to afford crude product.

General Procedure C (Addition of Di(2-Pyridyloxy)Methane-1-Thione):

A solution of starting amine (or amine-HCl with Et₃N) in THF or 1:1 THF:DMF is added drop wise to a stirred solution of di(2-pyridyloxy)methane-1-thione in THF. The reaction mixture is stirred about 2-6 h at room temperature and then solvent is removed under reduced pressure. The resulting residue is taken up in ethanol, the desired hydrazide is added and the reaction is heated on a 60° C. oil bath overnight. Excess ethanol is removed under reduced pressure to provide crude product.

General Procedure D (Boc Deprotection):

HCl in dioxane (4M) is added to a stirred solution of Boc-protected amine in CH₂Cl₂ and methanol and the resulting reaction is stirred at room temperature about 2-18 hours. Excess solvents are removed under reduced pressure. Et₂O is added and the resulting product suspension is briefly sonicated and then filtered to provide free amine products as HCl salts.

General Procedure E (Phthalimide Addition):

A solution of the desired alcohol and resin bound triphenyl-phosphine in THF is treated phthalimide and diisopropy-azo-dicarboxylate. The resulting solution is allowed to stir at room temperature about 2-18 hours. Resin is removed by filtration and washed in succession with THF, EtOAc, CH₂Cl₂, EtOAc, and methanol. Filtrate is dried with a rotoevaporator and the residue is taken up in EtOAc, washed with water, brine, then dried over magnesium sulfate, filtered and solvent is removed under reduced pressure to provide crude material.

General Procedure F (LiAlH₄ Reduction):

A solution of the desired ester or carboxylic acid in THF is cooled on an ice bath and lithium aluminum hydride is added. The reaction is either stirred at room temperature or heated on a 60° C. oil bath for about 2-18 hours. After complete consumption of starting material, a cooled reaction solution is diluted with Et₂O. Excess lithium aluminum hydride is quenched by the slow addition of a 1:1 mixture of sodium sulfate decahydrate and celite, stirring until the grey color of the solution clears and gas evolution ceases. The solution is filtered, dried and solvent is removed under reduced pressure to afford crude product.

General Procedure G (Boc Protection):

The desired amino acid, tert-butyl, (tert-butoxycarbonyloxy)formate, and sodium hydroxide are combined in water and stirred at room temperature overnight. Upon complete consumption of starting material, reaction mixture is diluted with water, pH adjusted to ˜3 with citric acid and product is extracted with EtOAc. Organic fractions are combined and washed with water and saturated sodium chloride, dried over magnesium sulfate, filtered and solvent removed under reduced pressure to afford crude product.

General Procedure H (Hydrazide Formation):

Hydrazine is added to the desired ester in ethanol and the resulting mixture is heated in a sealed tube on a 100° C. aluminum block about 2-18 hours, until starting material is fully consumed. Ethanol and excess hydrazine are removed under reduced pressure to provide the desired hydrazide product.

General Procedure I (Ester Formation):

A solution of the desired carboxylic acid in methanol is treated with sulfuric acid and heated to reflux overnight. Upon complete consumption of starting material, the cooled reaction mixture is poured into water, pH adjusted to ˜6 with ammonium hydroxide and product is extracted with CH₂Cl₂. Organic extracts are pooled and washed with water and saturated sodium chloride in succession, dried over magnesium sulfate, filtered and solvent removed under reduced pressure to provide the desired ester.

General Procedure J (Acid Formation from Nitrile):

The desired nitrile and concentrated hydrochloric acid are combined and heated on a 130° C. aluminum block overnight. Upon complete consumption of starting material, excess HCl is removed under reduced pressure. The resulting material is diluted with water, and the pH adjusted to ˜6 with ammonium hydroxide. Product is removed by filtration and dried under vacuum.

### 5.1.1 SYNTHESIS OF [2-AMINO-1-(2-PHENYLETHYL)ETHYL](5-(6-ISOQUINOLYL)(1,3,4-THIADIAZOL-2-YL))AMINE

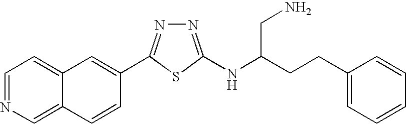

A. [2-Amino-1-(2-phenylethyl)ethyl](5-(6-isoquinolyl)(1,3,4-thiadiazol-2-yl))amine. N-{[({1-[(1,3-Dioxobenzo[c]azolidin-2-yl)methyl]-3-phenylpropyl}amino)thioxomethyl]amino}-6-isoquinolylcarboxamide (120 mg, 0.23 mmol) was cyclized according to General Procedure B to give a yellow solid, (95 mg, 82% yield). The phthalimide group was deprotected according to General Procedure A to give the amine. The crude product was further purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.9% purity, (35 mg, 50%). ¹H NMR (400 MHz, DMSO-d₆) δ 9.27 (s, 1H), 8.49 (d, J=5.6 Hz, 1H), 8.20 (s, 1H), 8.12 (m, 2H), 7.86 (d, J=9.6 Hz, 1H), 7.16 (m, 5H), 3.62 (m, 1H), 2.70 (d, J=5.6 Hz, 2H), 2.62 (m, 2H), 1.89 (m, 1H), 1.77 (m, 1H). MS (ESI) m/z 376.3 [M+1]⁺.

B. N-{[({1-[(1,3-Dioxobenzo[c]azolidin-2-yl)methyl]-3-phenylpropyl}amino)thioxo methyl]amino}-6-isoquinolylcarboxamide. N-[2-Amino-1-(2-phenylethyl)ethyl](tert-butoxy)carboxamide HCl (167 mg, 0.51 mmol) was coupled with isoquinoline-6-carbohydrazide according to General Procedure C. The crude product was precipitated out of ether to give an off-white solid, (240 mg, 86%). MS (ESI) m/z 524.5 [M+1]⁺.

C. N-[2-Amino-1-(2-phenylethyl)ethyl](tert-butoxy)carboxamide HCl. Racemic (tert-butoxy)-N-[2-hydroxy-1-(2-phenylethyl)ethyl]carboxamide (1.3 g, 4.9 mmol) underwent Mitsunobu reaction condition according to General Procedure E to give the phthalimide. The crude reaction was concentrated under reduced pressure to give an oil. The oil was treated with 4N HCl in dioxane and stirred at room temperature for 1 hour. until the Boc group was removed. The solid was triturated with EtOAc, filtered, and dried under vacuum to give a white solid, (0.8 g, 49% yield). MS (ESI) m/z 295.1 [M+1]⁺.

D. (tert-Butoxy)-N-[2-hydroxy-1-(2-phenylethyl)ethyl]carboxamide. 2-[(tert-Butoxy)carbonyl amino]-4-phenylbutanoic acid (5.9 g, 21 mmol) was dissolved in THF (40 mL). Ethyl chloroformate (2 mL, 21 mmol) and N-methylmorpholine (2.2 mL, 21 mmol) was added. The reaction was stirred for 10 minutes follow with addition of NaBH₄ in small portions. Then MeOH (100 mL) was added slowly to the reaction via an addition funnel over 40 minutes. The reaction was concentrated under reduced pressure to give a thick oil. The oil was flash chromatographed with 20%-60% EtOAc-hexane to give the alcohol, (5.5 g, 98% yield).

E. 2-[(tert-Butoxy)carbonylamino]-4-phenylbutanoic acid. DL-Homopheylalanine (5 g, 28 mmol) was added with dichloromethane (100 mL) and potassium hydroxide (12.5 g, 223 mmol) dissolved in water (100 mL). The reaction mixture was stirred vigorously while di-tert-butyl dicarbonate (6.1 g, 28 mmol) dissolved in dichloromethane (20 mL) was added. The biphasic mixture was stirred at room temperature for 2 hours until completion. The reaction was adjusted to pH 3 with 1M HCl and then extracted with dichloromethane. The organic layer was further extracted with water and brine before drying over MgSO₄. The dichloromethane mixture was filtered and dried to give a solid foam, (5.9 g, 76%). MS (ESI) m/z 280.3 [M+1]⁺.

### 5.1.2 SYNTHESIS OF [2-AMINO-1-BENZYLETHYL](5-(6-ISOQUINOLYLY)(1,3,4-THIADIAZOL-2-YL))AMINE

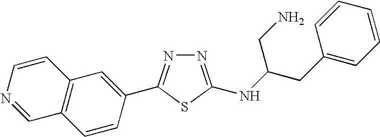

A. [2-Amino-1-benzylethyl](5-(6-isoquinolyl)(1,3,4-thiadiazol-2-yl))amine. A solution of 2-{2-[(5-(6-isoquinolyl)(1,3,4-thiadiazol-2-yl))amino]-3-phenylpropyl}benzo[c]azoline-1,3-dione (0.2 g, 0.4 mmol) and hydrazine (0.1 mL, excess) in ethanol (15 mL) were reacted according to General Procedure A. Product was purified using reverse-phase semi-preparatory HPLC (10-40% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.6% purity, (55 mg, 18%). ¹H NMR (400 MHz, d₆-DMSO) δ 9.33 (s, 1H), 8.55 (d, J=5.65, 1H), 8.24 (s, 1H), 8.14 (ABq, J=8.62, 2H), 7.92 (d, J=5.79, 1H), 7.34-7.16 (overlapping m, 5H), 3.87 (m, 1H), 2.91 (ABX, J_{AB}=13.52, J_{AX}=6.39, J_{BX}=6.91, 2H), 2.71 (ABX, J_{AB}=13.07, J_{AX}=5.20, J_{BX}=6.08, 2H); MS (ESI) m/z 362.0 [M+1]⁺.

B. 2-{2-[(5-(6-isoquinolyl)(1,3,4-thiadiazol-2-yl))amino]-3-phenylpropyl}benzo[c]azoline-1,3-dione. Methane sulfonic acid (1.5 mL, excess) and N-[({[2-(1,3-dioxobenzo[c]azolin-2-yl)-1-benzylethyl]amino}thioxomethyl)amino]-6-isoquinolylcarboxamide (0.25 g, 0.5 mmol) were reacted as described in General Procedure B. Purification using silica gel flash column chromatography (10% methanol in CH₂Cl₂) provided the title compound, (0.213 g, 88%). MS (ESI) m/z 492.5 [M+1]⁺.

C. N-[({[(1R)-2-(1,3-Dioxobenzo[c]azolin-2-yl)-1-benzylethyl]amino}thioxomethyl)amino]-6-isoquinolylcarboxamide. The HCl salt of 2-(2-amino-3-phenylpropyl)benzo[c]azoline-1,3-dione (0.25 g, 0.8 mmol), triethylamine (0.4 mL, 2.8 mmol), di(2-pyridyloxy)methane-1-thione (0.19 g, 0.8 mmol), and isoquinoline-6-carbohydrazide (0.16 g, 0.8 mmol) were reacted according to General Procedure C. Purification using Biotage column chromatography (0-40% methanol in CH₂Cl₂) provided clean product (0.279 g, 69%). MS (ESI) m/z 510.5 [M+1]⁺.

### 5.1.3 SYNTHESIS OF {2-AMINO-1-[(2-CHLOROPHENYL)METHYL]ETHYL}(5-(6-ISOQUINOLYLY)(1,3,4-THIADIAZOL-2-YL))AMINE

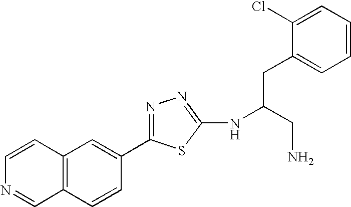

A. {2-Amino-1-[(2-chlorophenyl)methyl]ethyl}(5-(6-isoquinolyl)(1,3,4-thiadiazol-2-yl))amine. A solution of N-{[({2-(1,3-dioxobenzo[c]azolidin-2-yl)-1-[(2-chlorophenyl)methyl]ethyl}amino)thioxomethyl]amino}-6-isoquinolylcarboxamide (488 mg, 0.897 mmol) in methanesulfonic acid (4 mL) was stirred about 18 hours at room temperature. The reaction was diluted with water, chilled in an ice bath, and neutralized with saturated aqueous sodium bicarbonate. The resulting solution was extracted 3 times with dichloromethane. The organic solution was dried over anhydrous sodium sulfate, filtered, and the volatiles evaporated. The resulting crude intermediate (378 mg) was dissolved in DMF (2 mL) and hydrazine monohydrate (3 mL) was added. The reaction was stirred for about 2 hours at room temperature. The volatiles were evaporated and the resulting material was purified using reverse-phase preparatory HPLC (10-60% acetonitrile/water/0.1% TFA, over 30 minutes). Fractions containing clean product were combined and the solution made basic with aqueous ammonium hydroxide. The acetonitrile was evaporated and the resulting solution was extracted 5 times with 10% methanol in dichloromethane. The organic solution was dried over anhydrous sodium sulfate, filtered, and volatiles evaporated. The material was dried under vacuum at 60° C. to give the title compound (141.2 mg, 40%, 2 steps). ¹H NMR (400 MHz, DMSO) δ 9.32 (s, 1H), 8.55 (d, J=5.6, 1H), 8.23 (s, 1H), 8.18 (d, J=8.4, 1H), 8.13 (d, J=8.0, 1H), 7.92 (d, J=5.6, 1H), 7.42 (d, J=5.6, 2H), 7.20-7.30 (m, 2H), 3.95-4.05 (m, 1H), 3.10-3.15 (m, 1H), 2.90-2.96 (m, 1H), 2.70-2.85 (m, 2H); MS (ESI) m/z 396.1 [M+1]⁺.

B. N-{[({2-(1,3-Dioxobenzo[c]azolidin-2-yl)-1-[(2-chlorophenyl)methyl]ethyl}amino)thioxomethyl]amino}-6-isoquinolylcarboxamide. To a suspension of 2-[2-amino-3-(2-chlorophenyl)propyl]benzo[c]azolidine-1,3-dione hydrochloride (530 mg, 1.51 mmol) in anhydrous THF (12 mL) was added anhydrous triethylamine (0.230 mL, 1.66 mmol) and di-2-pyridyl thionocarbonate (367 mg, 1.58 mmol). The reaction was stirred one hour at room temperature and then filtered. The volatiles were evaporated from the filtrate and the residue was dried under vacuum at room temperature. To a solution of the residue in anhydrous ethanol (18 mL) was added isoquinoline-6-carbohydrazide (295 mg, 1.05 mmol) and the mixture heated to 60° C. with stirring for one hour. The reaction mixture was cooled to room temperature and the resulting precipitate was filtered and rinsed with ethyl acetate. The material was dried under vacuum to give the title compound (488 mg, 59%). MS (ESI) m/z 544.3 [M+1]⁺.

C. 2-[2-Amino-3-(2-chlorophenyl)propyl]benzo[c]azolidine-1,3-dione hydrochloride. To a solution of (tert-butoxy)-N-{1-[(2-chlorophenyl)methyl]-2-hydroxyethyl}carboxamide (2.67 g, 9.34 mmol) in anhydrous THF (47 mL) was added phthalimide (1.512 g, 10.27 mmol), polymer-supported triphenylphosphine (3.74 g, 11.21 mmol, 3 mmol P/g). The mixture was chilled in a ice water bath, diisopropyl azodicarboxylate (1.84 mL, 9.34 mmol) was added drop wise, and the mixture was stirred for 24 hours at room temperature. The reaction was filtered and the solids washed in succession with THF, dichloromethane, ethyl acetate, and methanol. The volatiles were evaporated from the filtrate and the residue was dried under vacuum at room temperature. The residue was dissolved in dichloromethane, the solution cooled, and the precipitate filtered. The material was dried under vacuum to give crude intermediate (993 mg) MS (ESI) m/z 415.4 [M+1]⁺. The material was then dissolved in a solution of hydrogen chloride in dioxane (20 mL, 4N) and the reaction was stirred for 3 hours at room temperature. The reaction was diluted with ethyl ether and the resulting precipitate was filtered and rinsed with ethyl ether. The material was dried under vacuum to give the title compound as a white solid (695 mg, 18%). MS (ESI) m/z 315 [M+1]⁺.

D. (tert-Butoxy)-N-{1-[(2-chlorophenyl)methyl]-2-hydroxyethyl}carboxamide. To a ice cold solution of (2R)-2-[(tert-butoxy)carbonylamino]-3-(2-chlorophenyl)propanoic acid (1.5 g, 10.0 mmol) and (2S)-2-[(tert-butoxy)carbonylamino]-3-(2-chlorophenyl)propanoic acid (1.5 g, 10.0 mmol) in anhydrous THF (30 mL) was added 4-methylmorpholine (1.10 mL, 10 mmol) and ethyl chloroformate (0.956 mL, 10 mmol). The mixture was stirred for 10 minutes and then sodium borohydride (1.13 g, 30 mmol) was added in portions, followed by drop wise addition of anhydrous methanol (150 mL) over 30 minutes. The mixture was stirred for an additional 30 minutes and then aqueous potassium hydrogen sulfate (100 mL, 1M), and ethyl acetate (300 mL) were added. The resulting precipitate was filtered and the filtrate was washed with aqueous sodium bicarbonate, water, and brine. The organic phase was dried over anhydrous sodium sulfate, filtered, and volatiles evaporated. The material was dried under vacuum at 60° C. to give the title compound (2.67 g, 93%). MS (ESI) m/z 286.3 [M+1]⁺.

### 5.1.4 SYNTHESIS OF {2-AMINO-1-[(3-CHLOROPHENYL)METHYL]ETHYL}(5-(6-ISOQUINOLYL)(1,3,4-THIADIAZOL-2-YL))AMINE

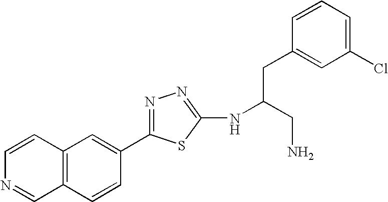

A. {2-Amino-1-[(3-chlorophenyl)methyl]ethyl}(5-(6-isoquinolyl)(1,3,4-thiadiazol-2-yl))amine. The compound was prepared as in Example 5.1.3, Step A, using N-{[({2-(1,3-dioxobenzo[c]azolidin-2-yl)-1-[(3-chlorophenyl)methyl]ethyl}amino)thioxomethyl]amino}-6-isoquinolylcarboxamide (464 mg, 0.852 mmol) to give the title compound (177 mg, 52%, 2 steps). ¹H NMR (400 MHz, DMSO) δ 9.34 (s, 1H), 8.55 (d, J=5.6, 1H), 8.26 (s, 1H), 8.15-8.20 (m, 2H), 7.92 (d, J=5.6, 1H), 7.25-7.37 (m, 4H), 3.85-3.91 (m, 1H), 2.80-3.00 (m, 2H), 2.65-2.75 (m, 2H); MS (ESI) m/z 396.1 [M+1]⁺.

B. N-{[({2-(1,3-Dioxobenzo[c]azolidin-2-yl)-1-[(3-chlorophenyl)methyl]ethyl}amino)thioxomethyl]amino}-6-isoquinolylcarboxamide. The compound was prepared as in Example 5.1.3, Step B, using 2-[2-amino-3-(3-chlorophenyl)propyl]benzo[c]azolidine-1,3-dione hydrochloride (530 mg, 1.51 mmol) to give the title compound (464 mg, 57%). MS (ESI) m/z 544.3 [M+1]⁺.

C. 2-[2-Amino-3-(3-chlorophenyl)propyl]benzo[c]azolidine-1,3-dione hydrochloride. The compound was prepared as in Example 5.1.3, Step C, using (tert-Butoxy)-N-{1-[(3-chlorophenyl)methyl]-2-hydroxyethyl}carboxamide (2.71 g, 9.48 mmol) to give the title compound as a white solid (551 mg, 14%). MS (ESI) m/z 315 [M+1]⁺.

D. (Tert-butoxy)-N-{1-[(3-chlorophenyl)methyl]-2-hydroxyethyl}carboxamide. The compound was prepared as in Example 5.1.3, Step D, using (2R)-2-[(tert-butoxy)carbonylamino]-3-(3-chlorophenyl)propanoic acid (1.5 g, 10.0 mmol) and (2S)-2-[(tert-butoxy)carbonylamino]-3-(3-chlorophenyl)propanoic acid (1.5 g, 10.0 mmol) to give the title compound (2.71 g, 95%). MS (ESI) m/z 286.3 [M+1]⁺.

### 5.1.5 SYNTHESIS OF [3-AMINO-1-BENZYLPROPYL](5-(6-ISOQUINOLYLY)(1,3,4-THIADIAZOL-2-YL))AMINE

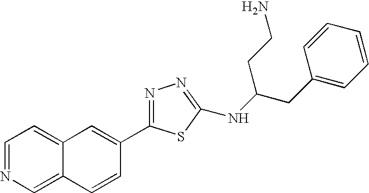

A. [3-amino-1-benzylpropyl](5-(6-isoquinolyl)(1,3,4-thiadiazol-2-yl))amine. To a solution of 2-{3-[(5-(6-isoquinolyl)(1,3,4-thiadiazol-2-yl))amino]-4-phenylbutyl}benzo[c]azoline-1,3-dione (0.730 g., 1.44 mmol) in dimethyl formamide (4 mL) was added hydrazine monohydrate (6 mL). The solution was monitored by thin layer chromatography. After one hour, solution was condensed under reduced pressure and the resultant oil purified via preparative HPLC (10-65% acetonitrile/water, 60 mL/min.) to afford the title compound (0.190 g., 35%). ¹H NMR (400 MHz, CD₃OD) δ 9.29 (s, 1H), 8.51 (d, J=5.99, 1H), 8.24 (s, 1H), 8.20 (s, 2H), 7.92 (d, J=5.59, 1H), 7.30 (m, 4H), 7.20 (m, 1H), 4.23 (m, 1H), 2.98 (d, 2H), 2.82 (m, 2H), 1.91 (m, 1H), 1.77 (m, 1H). MS (ESI) m/z 376 [M+1]⁺.

B. 2-{3-[(5-(6-isoquinolyl)(1,3,4-thiadiazol-2-yl))amino]-4-phenylbutyl}benzo[c azoline-1,3-dione. N-[({[3-(1,3-dioxobenzo[c]azolin-2-yl)-1-benzylpropyl]amino}thioxomethyl)amino]-6-isoquinolylcarboxamide (0.810 g., 1.55 mmol) was dissolved in methanesulfonic acid (10 mL) and allowed to stir at ambient temperature. Starting material consumption was monitored by LCMS. After four hours, the solution was basified with ammonium hydroxide and partitioned with dichloromethane (3×). The organics were dried over magnesium sulfate, filtered and solvent removed to afford the title compound (0.730 g., 93%). MS (ESI) m/z 506 [M+1]⁺.

C. N-[({[3-(1,3-dioxobenzo[c]azolin-2-yl)-1-benzylpropyl]amino}thioxomethyl)amino]-6-isoquinolylcarboxamide. 2-(3-amino-4-phenylbutyl)benzo[c]azolidine-1,3-dione (0.836 g., 2.53 mmol) and isoquinoline-6-carbohydrazide (0.473 g., 2.53 mmol) were reacted according to General Procedure C. Instead of condensing the mixture, the resultant precipitate was filtered and dried under vacuum oven conditions to afford the title compound (0.810 g., 61%). (ESI) m/z 524 [M+1]⁺.

D. 2-(3-amino-4-phenylbutyl)benzo[c]azolidine-1,3-dione. (Tert-butoxy)-N-[3-(1,3-dioxobenzo[c]azolidin-2-yl)-1-benzylpropyl]carboxamide (1.0 g., 2.53 mmol) and 4N HCl/Dioxane were reacted according to General Procedure D to afford the title compound as the HCl salt. (0.880 g., 100%). (ESI) m/z 295 [M+1]⁺.

E. (Tert-butoxy)-N-[3-(1,3-dioxobenzo[c]azolidin-2-yl)-1-benzylpropyl]carboxamide. (tert-butoxy)-N-[3-hydroxy-1-benzylpropyl]carboxamide (1.34 g., 5.05 mmol) and diisopropyl azodicarboxylate (1.53 g., 7.58 mmol) were reacted according to General Procedure E. The crude solid was purified via silica gel chromatography (20-25% ethyl acetate/hexanes) to afford the title compound (1.75 g., 85%). MS (ESI) m/z 395 [M+1]⁺.

F. (Tert-butoxy)-N-[3-hydroxy-1-benzylpropyl]carboxamide. To a solution containing Boc-L-Homophenylalanine (1.0 g, 3.58 mmol) and Boc-D-homophenylalanine in THF (25 mL) was added lithium aluminum hydroxide (0.516 g., 16.12 mmol) portionwise. The reaction was monitored by thin layer chromatography (50% ethyl acetate/hexanes) and potassium permanganate stain. After 15 minutes, the solution was diluted with diethyl ether (25 mL) and neutralized with a mixture of 1:1 sodium sulfate:decahydrate and celite, stirring until grey color of solution cleared and gas evolution ceased. The solution was filtered, dried and solvent removed under reduced pressure to afford crude product. Hexanes were added and the solution sonicated to give a white precipitate. The solution was then condensed under reduced pressure to afford the title compound (1.34 g., 71%). MS (ESI) m/z 266 [M+1]⁺.

### 5.1.6 SYNTHESIS OF (5-(6-ISOQUINOLYL)(1,3,4-THIADIAZOL-2-YL)) [2-(METHYLAMINO)-1-BENZYLETHYL]AMINE

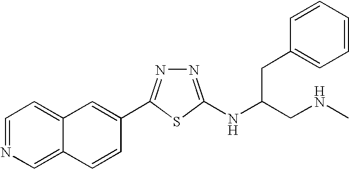

A. {2-[(5-(6-Isoquinolyl)(1,3,4-thiadiazol-2-yl))amino]-3-phenylpropyl}methylamine. 6-Isoquinolyl-N-[({[1-(methylamino)-2-phenylethyl]amino}thioxomethyl)amino]carboxamide (250 mg, 0.5 mmol) was deprotected according to General Procedure D to give the N-Boc product. The solid was cyclized according to General Procedure B. The crude product was further purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 97.8% purity, (150 mg, 79%). ¹H NMR (400 MHz, DMSO-d₆) δ 9.99 (s, 1H), 9.14 (bd, J=24.0 Hz, 1H), 8.72 (d, J=6.4 Hz, 1H), 8.62 (s, 1H), 8.58 (d, J=8.8 Hz, 1H), 8.54 (d, J=6.8 Hz, 1H), 8.46 (dd, J=8.8, 1.6 Hz, 1H), 4.45 (br, 1H), 3.19 (m, 2H), 3.03 (d, J=8.0 Hz, 2H), 2.59 (t, J=5.2 Hz, 3H). MS (ESI) m/z 376.3 [M+1]⁺.

B. 6-Isoquinolyl-N-[({[1-(methylamino)-2-phenylethyl]amino}thioxomethyl)amino]carboxamide. N-(2-Amino-3-phenylpropyl)(tert-butoxy)-N-methylcarboxamide (140 mg, 0.53 mmol) was coupled with isoquinoline-6-carbohydrazide according to General Procedure C. The crude product was precipitated out of ether to give a solid, (180 mg, 69%). MS (ESI) m/z 494.5 [M+1]⁺.

C. N-(2-Amino-3-phenylpropyl)(tert-butoxy)-N-methylcarboxamide. (tert-Butoxy)-N-(2-hydroxy-3-phenylpropyl)-N-methylcarboxamide (1.7 g, 6.4 mmol) was dissolved in THF (40 mL), triphenylphosphine (1.8 g, 7 mmol) and phthalimide (1 g, 7 mmol) were added together. The reaction was cooled in an ice-bath while diisopropyl azodicarboxylate (1.3 mL, 7 mL) was added dropwise via a syringe. The reaction was stirred at room temperature for 16 hours followed by removal of solvent under reduced pressure. The resultant residue was flash chromatographed with 5% EtOAc-hexane to give the crude phthalimide product. The crude product was taken up in EtOH (20 mL) and then added with hydrazine hydrate (2 mL). The reaction was stirred at room temperature for 16 hours followed by removal of volatile solvent at reduced pressure to give an oil. The oil was flash chromatographed with 10% MeOH-EtOAc to give the primary amine as an oil. MS (ESI) m/z 265.0 [M+1]⁺.

D. (tert-Butoxy)-N-(2-hydroxy-3-phenylpropyl)-N-methylcarboxamide. Benzyl ethylene oxide (2 g, 15 mmol), methylamine HCl (3 g, 45 mmol), K₂CO₃ (6 g, 45 mmol), and MeOH (15 mL) were heated together in a microwave (100° C., 10 min.). The reaction was filtered through celite and then concentrated to give a lightly yellow oil. The oil was flash chromatographed with 10% MeOH-EtOAc to isolate the mono-alkylated product, (1.1 g, 44%). MS (ESI) m/z 166.3 [M+1]⁺. The amino alcohol (1.1 g, 6.7 mmol) was taken up in 2-propanol (10 mL), added with di-tert-butyl dicarbonate (1.7 g, 8 mmol), and then stirred at room temperature for 1 h. The reaction was concentrated under reduce pressure and the product was used for the next step without further purification. MS (ESI) m/z 266.0 [M+1]⁺.

### 5.1.7 SYNTHESIS OF [2-(CYCLOPROPYLAMINO)-1-BENZYLETHYL](5-(6-ISOQUINOLYL)(1,3,4-THIADIAZOL-2-YL))AMINE

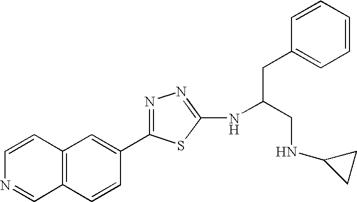

A. [2-(Cyclopropylamino)-1-benzylethyl](5-(6-isoquinolyl)(1,3,4-thiadiazol-2-yl))amine. To a solution of N-(2-amino-3-phenylpropyl)(tert-butoxy)-N-cyclopropylcarboxamide (163 mg, 0.561 mmol) in anhydrous THF (2 mL) was added a solution of di-2-pyridyl thionocarbonate (137 mg, 0.589 mmol) in anhydrous THF (4 mL) followed by anhydrous triethylamine (0.08 mL, 0.561 mmol). The reaction was stirred 20 minutes at room temperature and then the volatiles were evaporated. The residue was dried under vacuum at room temperature. To a solution of the residue in anhydrous ethanol (6 mL) was added isoquinoline-6-carbohydrazide (110 mg, 0.589 mmol) and the mixture heated to 60° C. with stirring for two hours. The reaction mixture was cooled to room temperature, diluted with anhydrous methanol (20 ml), and the solution of N-{[({2-[(tert-butoxy)-N-cyclopropylcarbonylamino]-1-benzylethyl}amino)thioxomethyl]amino}-6-isoquinolylcarboxamide was chilled in ice water. The solution was saturated with anhydrous hydrogen chloride, stirred 20 minutes in the ice bath, and then the volatiles were evaporated. The material was dried under vacuum at 60° C., dissolved in methane sulfonic acid (4 mL) heated to 60° C. for 45 minutes. The reaction mixture was neutralized with saturated aqueous sodium bicarbonate. The resulting solution was extracted seven times with a solution of 5% methanol in dichloromethane. The solvent was evaporated and the resulting material was purified using reverse-phase preparatory HPLC (10-50% acetonitrile/water/0.1% TFA, over 30 min). Fractions containing clean product were combined and the solution made basic with aqueous ammonium hydroxide. The acetonitrile was evaporated and the resulting solution was extracted 3 times with 5% methanol in dichloromethane. The organic solution was dried over anhydrous sodium sulfate, filtered, and volatiles evaporated. The material was dried under vacuum at 60° C. to give the title compound (76 mg, 33%, 4 steps). ¹H NMR (400 MHz, DMSO) δ 9.33 (s, 1H), 8.55 (d, J=6, 1H), 8.22 (s, 1H), 8.10-8.20 (m, 3H), 7.92 (d, J=6.0, 1H), 7.15-7.35 (m, 5H), 4.00-4.15 (m, 1H), 2.70-3.00 (m, 4H), 2.05-2.20 (broad s, 1H), 0.38 (s, 2H), 0.25 (s, 2H); MS (ESI) m/z 402.5 [M+1]⁺.

B. N-(2-Amino-3-phenylpropyl)(tert-butoxy)-N-cyclopropylcarboxamide. To a solution of (tert-Butoxy)-N-[2-(1,3-dioxobenzo[c]azolidin-2-yl)-3-phenylpropyl]-N-cyclopropylcarbox-amide (1.32 g, 3.14 mmol) in anhydrous DMF (6 mL) was added hydrazine monohydrate (9 mL). The reaction was stirred for three hours at room temperature. The volatiles were evaporated. The material was heated and sonicated in a solution of methanol (5 mL) in dichloromethane (20 mL). The resulting precipitate was filtered. The filtrate was concentrated and then purified using reverse-phase preparatory HPLC (10-60% acetonitrile/water/0.1% TFA, over 30 min). Fractions containing clean product were combined and the solution made basic with aqueous ammonium hydroxide. The acetonitrile was evaporated and the resulting solution was extracted five times with 5% methanol in dichloromethane. The organic solution was dried over anhydrous sodium sulfate, filtered, and volatiles evaporated. The material was dried under vacuum at room temperature to give the title compound. (163 mg, 18%). MS (ESI) m/z 291.4 [M+1]⁺.

C. (Tert-butoxy)-N-[2-(1,3-dioxobenzo[c]azolidin-2-yl)-3-phenylpropyl]-N-cyclopropyl carboxamide. To a solution of (tert-butoxy)-N-cyclopropyl-N-(2-hydroxy-3-phenylpropyl)carboxamide (1.93 g, 6.64 mmol) in anhydrous THF (33 mL) was added phthalimide (1.074 g, 7.30 mmol), polymer-supported triphenylphosphine (2.66 g, 7.97 mmol, 3 mmol P/g). The mixture was chilled in a ice water bath, diisopropyl azodicarboxylate (1.31 mL, 6.64 mmol) was added drop wise, and the mixture was stirred for 24 hours at room temperature. The reaction was filtered and the solids washed in succession with THF, dichloromethane, ethyl acetate, and methanol. The volatiles were evaporated from the filtrate and residue was dried under vacuum at room temperature. The residue was dissolved in dichloromethane and the resulting precipitate filtered. The filtrate was concentrated and then purified using chromatography on a normal phase silica gel column with 0 to 20% ethyl acetate in hexanes. Fractions containing clean product were combined and the solvent evaporated. The material was dried under vacuum at room temperature to provide the title compound as a white solid (1.32 g, 47%). MS (ESI) m/z 421.4 [M+1]⁺.

D. (tert-Butoxy)-N-cyclopropyl-N-(2-hydroxy-3-phenylpropyl)carboxamide. To a solution of (2,3-epoxypropyl)benzene (1.84 g, 13.71 mmol) in anhydrous methanol (45 mL) was added cyclopropylamine (9.4 mL, excess). The reaction tube was sealed and the contents stirred at 60° C. for three hours. The volatiles were evaporated and the material was purified using chromatography on a normal phase silica gel column with 3 to 5% methanol in dichloromethane. Fractions containing clean product were combined and the solvent evaporated. The material was dried under vacuum at room temperature. The resulting intermediate oil (1.28 g), MS (ESI) m/z 192.3 [M+1]⁺, was dissolved in anhydrous methanol (30 mL) and di-tert-butyl dicarbonate (1.45 g, 6.64 mmol) was added. The mixture was stirred for 5 hours at room temperature, the volatiles were evaporated and the material was dried under vacuum at room temperature to provide the title compound (1.93 g, 99%). MS (ESI) m/z 292.4 [M+1]⁺.

### 5.1.8 SYNTHESIS OF [(1R)-2-AMINO-1-BENZYLETHYL](5-(6-ISOQUINOLYLY)(1,3,4-THIADIAZOL-2-YL))AMINE

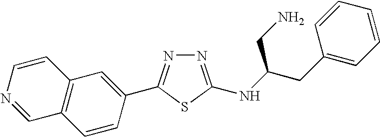

A. [(1R)-2-Amino-1-benzylethyl](5-(6-isoquinolyl)(1,3,4-thiadiazol-2-yl))amine. A solution of 2-{(2R)-2-[(5-(6-isoquinolyl)(1,3,4-thiadiazol-2-yl))amino]-3-phenylpropyl}benzo[c]azoline-1,3-dione (0.1 g, 0.2 mmol) and hydrazine (0.1 mL, excess) in ethanol (6 mL) were reacted according to General Procedure A. Product was purified using reverse-phase semi-preparatory HPLC (10-40% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.7% purity, (38 mg, 52%). ¹H NMR (400 MHz, CD₃OD) δ 9.26 (s, 1H), 8.49 (d, J=5.86, 1H), 8.22 (d, J=6.33, 2H), 8.18 (s, 1H), 7.89 (d, J=5.86, 1H), 7.31-7.18 (overlapping m, 5H), 4.09 (m, 1H), 2.97 (ABX, J_{AB}=14.06, J_{AX}=6.05, J_{BX}=4.19, 2H), 2.87 (ABX, J_{AB}=13.47, J_{AX}=7.42, J_{BX}=8.00, 2H); MS (ESI) m/z 362.0 [M+1]⁺. Anal. Calcd for C₂₀H₁₉N₅S, C, 65.96; H, 5.34; N, 19.23. Found, C, 65.57; H, 4.97; N, 19.23.

B. 2-{(2R)-2-[(5-(6-isoquinolyl)(1,3,4-thiadiazol-2-yl))amino]-3-phenylpropyl}benzo[c]azoline-1,3-dione. Methane sulfonic acid (1 mL, excess) and N—[({[(1R)-2-(1,3-dioxobenzo[c]azolin-2-yl)-1-benzylethyl]amino}thioxomethyl)amino]-6-isoquinolylcarboxamide (0.1 g, 0.2 mmol) were reacted as described in General Procedure B to provide pure material (0.095 g, 99%). MS (ESI) m/z 492.5 [M+1]⁺.

C. N-[({[(1R)-2-(1,3-Dioxobenzo[c]azolin-2-yl)-1-benzylethyl]amino}thioxomethyl)amino]-6-isoquinolylcarboxamide. The HCl salt of 2-((2R)-2-amino-3-phenylpropyl)benzo[c]azoline-1,3-dione (1.0 g, 3.2 mmol), triethylamine (1.0 mL, 7.2 mmol), di(2-pyridyloxy)methane-1-thione (0.74 g, 3.2 mmol), and isoquinoline-6-carbohydrazide (0.58 g, 3.2 mmol) were reacted according to General Procedure C. Purification using Biotage column chromatography (0-100% hexanes in EtOAc) provided clean product (1.15 g, 71%). MS (ESI) m/z 510.5 [M+1]⁺.

D. 2-((2R)-2-Amino-3-phenylpropyl)benzo[c]azoline-1,3-dione hydrochloride. N-[(1R)-2-(1,3-dioxobenzo[c]azolin-2-yl)-1-benzylethyl] (tert-butoxy)carboxamide (1.0 g, 2.6 mmol) and HCl (2 mL, 4M in dioxane, 8.0 mmol) in CH₂Cl₂ (100 mL) and methanol (10 mL) were reacted according to General Procedure D. The resulting white solid was dried under high vacuum with heating overnight to give clean product as an HCl salt (0.84 g, 100%). MS (ESI) m/z 281.1 [free base M+1]⁺.

E. N-[(1R)-2-(1,3-dioxobenzo[c]azolin-2-yl)-1-benzylethyl](tert-butoxy)carboxamide. N-[(1R)-2-hydroxy-1-benzylethyl] (tert-butoxy)carboxamide (2.0 g, 8.0 mmol), resin-bound triphenyl phosphine (4 g, 3 mmol P/g resin), phthalimide (1.6 g, 12 mmol) and diisopropy-azo-dicarboxylate (2.35 mL, 12 mmol) were reacted according to General Procedure E. Purification using Biotage column chromatography (0-50% EtOAc in hexanes) followed by recrystallization from CH₂Cl₂ and hexanes provided clean product (1.6 g, 70%). MS (ESI) m/z 381.5 [M+1]⁺.

F. Isoquinoline-6-carbohydrazide. Methyl isoquinoline-6-carboxylate (0.95 g, 5.1 mmol) and hydrazine (0.4 mL, 12.7 mmol) in ethanol (15 mL) were reacted as described in General Procedure H. The resulting product was dried under high vacuum to provide the title compound as a off white solid (0.90 g, 95%). MS (ESI) m/z 188.3 [M+1]⁺.

G. Methyl isoquinoline-6-carboxylate. A solution of isoquinoline-6-carboxylic acid (1 g, 5.8 mmol) and sulfuric acid (1 mL) in methanol (40 mL) were reacted according to General Procedure I. The resulting product was dried under vacuum to provide the title compound (0.98 g, 91%). MS (ESI) m/z 188.1 [M+1]⁺.

H. Isoquinoline-6-carboxylic acid. Isoquinoline-6-carbonitrile (2.5 g, 16.2 mmol) and HCl (15 mL) were reacted according to General Procedure J. Material was dried in vacuum oven overnight to give the title compound as a white solid (2.3 g, 82%). MS (ESI) m/z 174.2 [M+1]⁺.

### 5.1.9 SYNTHESIS OF [(1S)-2-AMINO-1-BENZYLETHYL](5-(6-ISOQUINOLYL)(1,3,4-THIADIAZOL-2-yl))AMINE

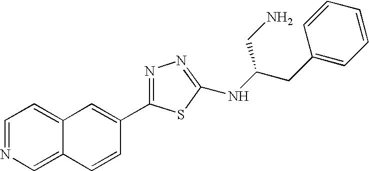

A. [(1S)-2-amino-1-benzylethyl](5-(6-isoquinolyl)(1,3,4-thiadiazol-2-yl))amine. 2-{(2S)-2-[(5-(6-isoquinolyl)(1,3,4-thiadiazol-2-yl))amino]-3-phenylpropyl}benzo[c]azoline-1,3-dione (0.340 g., 0.692 mmol) and hydrazine monohydrate (3 mL) were reacted according to General procedure A and purified via preparative HPLC (10-60% acetonitrile/water, 20 mL/min.) to afford the title compound (0.071 g., 28%). ¹H NMR (400 MHz, CD₃OD) δ 9.29 (s, 1H), 8.51 (d, J=5.59, 1H), 8.25 (s, 1H), 8.21 (s, 1H), 7.93 (d, J=5.19, 1H), 7.33-7.32 (overlapping m, 4H), 7.21 (m, 1H), 4.31 (m, 1H), 3.06-2.80 (overlapping m, 4H). MS (ESI) m/z 361 [M+1]⁺.

B. 2-{(2S)-2-[(5-(6-isoquinolyl)(1,3,4-thiadiazol-2-yl))amino]-3-phenylpropyl}benzo[c]azoline-1,3-dione. N-[({[(1S)-2-(1,3-dioxobenzo[c]azolin-2-yl)-1-benzylethyl]amino}thioxomethyl)amino]-6-isoquinolylcarboxamide (0.374 g., 0.734 mmol) and methanesulfonic acid (8 mL) were reacted according to Procedure B to afford the title compound (0.345 g., 96%). MS (ESI) m/z 492 [M+1]⁺.

C. N-[({[(1S)-2-(1,3-dioxobenzo[c]azolin-2-yl)-1-benzylethyl]amino}thioxomethyl)amino]-6-isoquinolylcarboxamide. 2-((2S)-2-amino-3-phenylpropyl)benzo[c]azoline-1,3-dione:HCl (0.350 g., 1.10 mmol) and isoquinoline-6-carbohydrazide (0.207 g., 1.107) were reacted according to Procedure C and purified via silica gel chromatography (90% ethyl acetate/hexanes) to afford the title compound (0.374 g., 66%). MS (ESI) m/z 510 [M+1]⁺.

D. 2-((2S)-2-amino-3-phenylpropyl)benzo[c]azoline-1,3-dione:HCl. N-[(1S)-2-(1,3-dioxobenzo[c]azolin-2-yl)-1-benzylethyl](tert-butoxy)carboxamide (0.500 g., 1.31 mmol), 4N HCl/Dioxane (4 mL) and MeOH (3 mL) were reacted according to Procedure D to afford the title compound (0.460 g., 100%). MS (ESI) m/z 281 [M+1]⁺.

E. N-[(1S)-2-(1,3-dioxobenzo[c]azolin-2-yl)-1-benzylethyl](tert-butoxy) carboxamide. (s)-(−)-2-(tert-butoxycarbonylamino-3-phenyl-1-propanol (2.5 g., 9.94 mmol) and phthalimide (2.19 g., 14.92 mmol) were reacted according to Procedure E and purified via silica gel chromatography (10-25% ethyl acetate/hexanes) to afford the title compound (3.35 g., 88%). MS (ESI) m/z 381 [M+1]⁺.

### 5.1.10 SYNTHESIS OF [2-AMINO-1-BENZYLETHYL](5-PYRIDINO[3,4-E]PYRIDIN-2-YL(1,3,4-THIADIAZOL-2-YL))AMINE

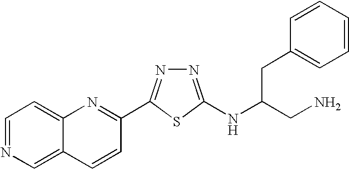

A. [2-Amino-1-benzylethyl](5-(6-isoquinolyl)(1,3,4-thiadiazol-2-yl))amine. N-[({[1-(1,3-Dioxobenzo[c]azolidin-2-yl)-2-phenylethyl]amino}thioxomethyl)amino]-6-isoquinolyl carboxamide (250 mg, 0.5 mmol) was cyclized according to General Procedure B to give a yellow solid. The phthalimide solid was deprotected according to General Procedure A to give the amine. The crude product was further purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.9% purity, (58 mg, 33%). ¹H NMR (400 MHz, DMSO-d₆) δ 9.41 (s, 1H), 8.76 (d, J=5.6 Hz, 1H), 8.64 (d, J=8.4 Hz, 1H), 8.32 (d, J=8.8 Hz, 1H), 7.88 (d, J=5.6 Hz, 1H), 7.29 (d, J=4.4 Hz, 4H), 7.18 (m, 1H), 3.90 (m, 1H), 2.96 (dd, J=13.6, 6.0 Hz, 1H), 2.73 (m, 2H). MS (ESI) m/z 363.0 [M+1]⁺.

B. N-[({[1-(1,3-dioxobenzo[c]azolidin-2-yl)-2-phenylethyl]amino}thioxomethyl)amino]-6-isoquinolylcarboxamide. 2-(2-Amino-3-phenylpropyl)benzo[c]azolidine-1,3-dione HCl (167 mg, 0.53 mmol) was coupled with isoquinoline-6-carbohydrazide according to General Procedure C. The crude product was precipitated out of ether to give an off-white solid, (190 mg, 70%). MS (ESI) m/z 511.4 [M+1]⁺.

C. 2-(2-Amino-3-phenylpropyl)benzo[c]azolidine-1,3-dione HCl.

Racemic (tert-butoxy)-N-[2-hydroxy-1-benzylethyl]carboxamide (1.5 g, 6 mmol) underwent Mitsunobu reaction condition according to General Procedure E to give the phthalimide. The crude reaction was concentrated under reduced pressure to give an oil. The oil was treated with 4N HCl in dioxane and stirred at room temperature for one hour. Then it was triturated with EtOAc to give a white solid. The solid was filtered and dried under vacuum to give the primary amine, (550 mg, 29% yield). MS (ESI) m/z 281.3 [M+1]⁺.

### 5.1.11 SYNTHESIS OF {2-[(5-(6-ISOQUINOLYL)(1,3,4-THIADIAZOL-2-YL))AMINO]-3-PHENYLPROPYL}DIMETHYLAMINE

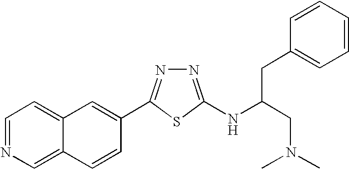

A. {2-[(5-(6-Isoquinolyl)(1,3,4-thiadiazol-2-yl))amino]-3-phenylpropyl}dimethylamine. N-[({[1-(Dimethylamino)-2-phenylethyl]amino}thioxomethyl)amino]-6-isoquinolylcarboxamide (110 mg, 0.27 mmol) was cyclized according to General Procedure B. The crude product was further purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.9% purity, (55 mg, 52%). ¹H NMR (400 MHz, DMSO-d₆) δ 9.99 (br, 1H), 9.76 (s, 1H), 8.75 (d, J=8.4 Hz, 1H), 8.68 (d, J=6.4 Hz, 1H), 8.54 (s, 1H), 8.49 (d, J=8.8 Hz, 1H), 8.39 (m, 2H), 7.30 (m, 4H), 7.20 (tt, J=8.8, 1.6 Hz, 1H), 4.55 (br, 1H), 3.40 (m, 2H), 3.02 (m, 2H), 2.86 (s, 3H), 2.85 (s, 3H). MS (ESI) m/z 390.5 [M+1]⁺.

B. N-[({[1-(Dimethylamino)-2-phenylethyl]amino}thioxomethyl)amino]-6-isoquinolyl carboxamide. (1-Amino-2-phenylethyl)dimethylamine (100 mg, 0.56 mmol) was coupled with isoquinoline-6-carbohydrazide according to General Procedure C. The crude product was precipitated out of ether to give an off-white solid, (110 mg, 48%). MS (ESI) m/z 408.5 [M+1]⁺.

C. (1-Amino-2-phenylethyl)dimethylamine. 1-(Dimethylamino)-2-phenylethan-1-ol was dissolved in THF (40 mL), and then added with triphenylphosphine (1.9 g, 7.4 mmol) and phthalimide (1.1 g, 7.4 mmol). The reaction was cooled in an ice-bath before addition of diisopropyl azodicarboxylate (1.56 mL, 7.4 mL) via a syringe. The reaction was stirred at room temperature for 16 hours followed by removal of solvent under reduced pressure. The resultant residue was flash chromatographed with 50% EtOAc-hexane to give the crude product. The crude product was added with hydrazine hydrate (2 mL) in EtOH (20 mL) and then heated to 60° C. for 16 hours. The reaction was concentrated down to an oil, and then flash chromatographed with 10% MeOH-EtOAc to give the primary amine as an oil, (0.5 g, 42%). MS (ESI) m/z 179.1 [M+1]⁺.

D. 1-(Dimethylamino)-2-phenylethan-1-ol. Benzyl ethylene oxide (1 g, 7.5 mmol), dimethylamine HCl (907 mg, 11 mmol), K₂CO₃ (3.1 g, 22 mmol), and MeOH (10 mL) were heated together in a microwave (100° C., 10 min.). The reaction was filtered through celite and then concentrated to give a lightly yellow oil, (1.2 g, 92%). MS (ESI) m/z 179.9 [M+1]⁺.

### 5.1.12 SYNTHESIS OF [2-AMINO-1-BENZYLETHYL](5-(6-ISOQUINOLYL)IMIDAZOL-2-YL)AMINE

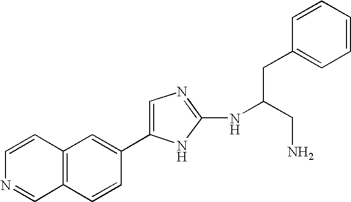

A. [2-Amino-1-benzylethyl](5-(6-isoquinolyl)imidazol-2-yl)amine. A solution of bis[(4-methoxyphenyl)methyl][2-({1-[(3,3-dimethyl-3-silabutoxy)methyl]-5-(6-isoquinolyl)imidazol-2-yl}amino)-3-phenylpropyl]amine (220 mg, 0.308 mmol) in trifluoroacetic acid (1 mL) was heated with stirring for 48 hours at 130° C. and then the volatiles were evaporated. The residue was taken up in methanol and the solution was filtered through 0.45 μm syringe filters, the filters were rinsed with methanol, and the filtrate was evaporated. The resulting material was purified using reverse-phase preparatory HPLC (10-60% acetonitrile/water/0.1% TFA, over 30 min). Fractions containing clean product were combined and the solution made basic with aqueous ammonium hydroxide. The acetonitrile was evaporated and the resulting solution was extracted 3 times with 5% methanol in dichloromethane. The organic solution was washed with water, dried over anhydrous sodium sulfate, filtered, and volatiles evaporated. The material was dried under vacuum to give the title compound (20 mg, 19%). ¹H NMR (400 MHz, DMSO) δ 9.14 (s, 1H), 8.39 (d, J=6, 1H), 8.05 (s, 1H), 7.97 (s, 2H), 7.71 (d, J=6.0, 1H), 7.25-7.35 (m, 5H), 7.15-7.20 (m, 1H), 5.78 (d, J=8.0, 1H), 3.70-3.80 (m, 1H), 2.78-2.90 (m, 2H), 2.58-2.70 (m, 2H); MS (ESI) m/z 344.3 [M+1]⁺.

B. Bis[(4-methoxyphenyl)methyl][2-({1-[(3,3-dimethyl-3-silabutoxy)methyl]-5-(6-isoquinolyl)imidazol-2-yl}amino)-3-phenylpropyl]amine. To a ice cold solution of (2-amino-3-phenylpropyl)bis[(4-methoxyphenyl)methyl]amine (0.617 mg, 1.58 mmol) in anhydrous dioxane (1.5 mL) was added n-butyllithium (0.966 mL, 1.55 mmol, 1.6 M in hexanes) drop wise with stirring. The reaction was stirred in an ice bath for 30 minutes, then an ice cold solution of 1-[(3,3-dimethyl-3-silabutoxy)methyl]-5-(6-isoquinolyl)-2-(phenylsulfonyl)imidazole (400 mg, 0.86 mmol) in anhydrous dioxane (1.0 mL) was added. The reaction was stirred in an ice bath for two hours followed by the addition of methanol, aqueous ammonium hydroxide, and water. The resulting mixture was extracted five times with dichloromethane, the combined organic phases were dried over anhydrous sodium sulfate. The solution was filtered, volatiles evaporated, and the residue purified using reverse-phase preparatory HPLC (30-80% acetonitrile/water/0.1% TFA, over 30 min). Fractions containing clean product were combined and the solution made basic with aqueous ammonium hydroxide. The acetonitrile was evaporated and the resulting solution was extracted five times with dichloromethane. The organic solution was dried over anhydrous sodium sulfate, filtered, and the volatiles were evaporated. The compound was dissolved in toluene and then the solvent was evaporated. The material was dried under vacuum at room temperature to give the title compound (243 mg, 40%). MS (ESI) m/z 714.7 [M+1]⁺.

C. (2-Amino-3-phenylpropyl)bis[(4-methoxyphenyl)methyl]amine. A solution of N-(2-{bis[(4-methoxyphenyl)methyl]amino}-1-benzylethyl)(tert-butoxy)carboxamide (775 mg, 1.58 mmol) in ethyl acetate (20 mL) was chilled in a ice water bath. The solvent was saturated with anhydrous hydrogen chloride gas and then stirred at room temperature for 30-60 minutes. The volatiles were evaporated, the resulting material was dissolved in dichloromethane (5 mL), and the solution was mixed with aqueous potassium carbonate. The organic phase was separated and the aqueous solution extracted three times with dichloromethane. The combined extracts were dried over anhydrous sodium sulfate, filtered, and volatiles evaporated. The compound was dissolved in toluene and then the solvent was evaporated. The material was dried under vacuum at room temperature to give the title compound. (617 mg, 99%). MS (ESI) m/z 391.5 [M+1]⁺.

D. 1-[(3,3-Dimethyl-3-silabutoxy)methyl]-5-(6-isoquinolyl)-2-(phenylsulfonyl)imidazole. To a solution of 6-bromoisoquinoline (73 mg, 0.35 mmol) in anhydrous DMF (0.75 mL) was added copper(II) oxide (28 mg, 0.35 mmol). The mixture was purged with nitrogen, tetrakis(triphenylphosphine)palladium(0) (55 mg, 0.048 mmol) was added, and then the materials were stirred at 100° C. for 5 minutes in a sealed vial. The vial was cooled to room temperature and then a solution of 5-(1,1-dibutyl-1-stannapentyl)-1-[(3,3-dimethyl-3-silabutoxy)methyl]-2-(phenylsulfonyl)imidazole (200 mg, 0.3.19 mmol) in anhydrous DMF (0.25 mL) was added. The mixture was purged with nitrogen then the sealed reaction was stirred at 105° C. for 8 hours. The reaction was cooled to room temperature, filtered through celite, and the celite rinsed with ethyl acetate. The filtrate was diluted with water, extracted three times with dichloromethane, and the organic layers combined. The organic solution was washed with brine, dried over anhydrous magnesium sulfate, filtered, and volatiles evaporated. The material was purified by chromatography on a normal phase silica gel column with 30 to 100% ethyl acetate in hexanes. Fractions containing clean product were combined and the solvent evaporated. The material was dried under vacuum at 60° C. to provide the title compound (1.2 g, 87%). MS (ESI) m/z 466.2 [M+1]⁺.

E. 5-(1,1-Dibutyl-1-stannapentyl)-1-[(3,3-dimethyl-3-silabutoxy)methyl]-2-(phenyl sulfonyl)imidazole. A solution of 1-[(3,3-dimethyl-3-silabutoxy)methyl]-5-(6-isoquinolyl)-2-(phenylsulfonyl)imidazole (1.75 g, 5.17 mmol) in anhydrous THF (51 mL) was chilled in a acetone/dry ice bath and then n-butyllithium (3.81 mL, 6.10 mmol, 1.6 M in hexanes) was added drop wise with stirring. The mixture was stirred at −78° C. for 30 minutes and then tributyltin chloride (1.40 mL, 5.17 mmol) was added drop wise with stirring. The reaction was stirred at room temperature for 4 hours, quenched with water (10 mL), and the volatiles evaporated. The residue was taken up in ethyl ether, washed three times with saturated aqueous ammonium chloride, and once with brine. The organic solution was dried over anhydrous sodium sulfate, filtered, and solvent evaporated. The material was purified using chromatography on a normal phase silica gel column with 0 to 10% ethyl acetate in hexanes. Fractions containing clean product were combined and the solvent evaporated. The material was dried under vacuum at room temperature to provide the title compound as an oil (1.907 g, 59%).

F. 1-[(3,3-Dimethyl-3-silabutoxy)methyl]-5-(6-isoquinolyl)-2-(phenylsulfonyl)imidazole. A solution of 1-(imidazolylmethoxy)-3,3-dimethyl-3-silabutane (1.48 g, 7.50 mmol) in anhydrous THF (75 mL) was chilled in a acetone/dry ice bath and then n-butyllithium (6.0 mL, 9.6 mmol, 1.6 M in hexanes) was added drop wise with stirring. The mixture was stirred at −78° C. for 30 minutes and then a solution of phenyl disulfide (2.1 g, 9.6 mmol) in anhydrous THF (2 mL) was added drop wise with stirring. The reaction was stirred in a ice water a bath for 1 hour and then at room temperature for 1 hour. The reaction was quenched with water (5 mL), the solvent evaporated, and the residue was taken up in ethyl ether. The organic solution was washed three times with aqueous sodium bicarbonate and once with brine. The organic solution was dried over anhydrous sodium sulfate, filtered, and solvent evaporated. The material was purified using chromatography on a normal phase silica gel column with 10 to 20% ethyl acetate in hexanes. Fractions containing clean product were combined and the solvent evaporated. The material was dried under vacuum at room temperature to provide the intermediate 3,3-dimethyl-1-[(2-phenylthioimidazolyl)methoxy]-3-silabutane (1.76 g). A solution of the intermediate in anhydrous dichloromethane (26 mL) was purged with nitrogen, 3-chloroperoxybenzoic acid (3.2 g, 14.28 mmol) was added, and then the reaction was stirred at room temperature. After 18 hours, sodium thiosulfate penta hydrate (1.125 g) was added. The mixture was stirred at room temperature for 15 minutes and filtered. The filtrate was washed three times with a solution of 5% aqueous sodium carbonate and once with brine. The organic solution was dried over anhydrous sodium sulfate, filtered, and volatiles evaporated. The compound was dissolved in toluene and then the solvent was evaporated. The material was dried under vacuum at room temperature to provide the title compound as an oil (1.75 g, 69%, 2 steps). MS (ESI) m/z 339.3 [M+1]⁺.

G. 1-(Imidazolylmethoxy)-3,3-dimethyl-3-silabutane. A suspension of sodium hydride (3.24 g, 81.0 mmol, 60% in mineral oil) in hexanes (150 mL) was stirred for a few minutes at room temperature and then the solids allowed to settle to the bottom of the flask. The hexanes were decanted and discarded. The sodium hydride was washed two more times with hexanes and then dried under vacuum at room temperature. The sodium hydride was dissolved in DMF (150 mL), the solution chilled to −20 to −15° C., and then imidazole (5.00 g, 73.4 mmol) was added, in small portions, with stirring. The mixture was stirred at room temperature for 30 minutes then 2-(trimethylsilyl)ethoxymethyl chloride (13.23 mL, 75 mmol) was added drop wise with stirring. The reaction was stirred at room temperature for 1 hour and then water (10 mL) was added drop wise followed by methanol (2 mL). The volatiles were evaporated and the residue was taken up in ethyl ether. The organic solution was washed five times with and once with brine. The organic solution was dried over anhydrous sodium sulfate, filtered, and solvent evaporated. The compound was dissolved in toluene and then the solvent was evaporated. The material was dried under vacuum at room temperature to provide the title compound as an oil (8.25 g, 57%). MS (ESI) m/z 199 [M+1]⁺.

### 5.1.13 SYNTHESIS OF {2-AMINO-1-[(4-CHLOROPHENYL)METHYL]ETHYL}(5-(6-ISOQUINOLYL)(1,3,4-THIADIAZOL-2-YL))AMINE

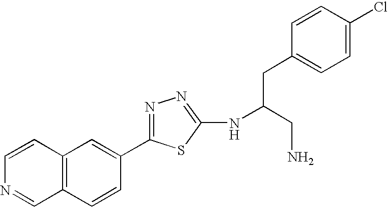

A. {2-Amino-1-[(4-chlorophenyl)methyl]ethyl}(5-(6-isoquinolyl)(1,3,4-thiadiazol-2-yl))amine. The compound was prepared as in Example 5.1.3, Step A, using N-{[({2-(1,3-dioxobenzo[c]azolidin-2-yl)-1-[(4-chlorophenyl)methyl]ethyl}amino)thioxomethyl]amino}-6-isoquinolylcarboxamide (450 mg, 0.827 mmol) to give the title compound (165 mg, 50%, 2 steps). ¹H NMR (400 MHz, DMSO) δ 9.33 (s, 1H), 8.55 (d, J=6, 1H), 8.22 (s, 1H), 8.12-8.20 (m, 2H), 7.92 (d, J=6.0, 1H), 7.30-7.40 (m, 4H), 3.80-3.90 (m, 1H), 2.80-3.00 (m, 2H), 2.65-2.78 (m, 2H); MS (ESI) m/z 396.1 [M+1]⁺.

B. N-{[({2-(1,3-Dioxobenzo[c]azolidin-2-yl)-1-[(4-chlorophenyl)methyl]ethyl}amino)thioxomethyl]amino}-6-isoquinolylcarboxamide. The compound was prepared as in Example 5.1.3, Step B, using 2-[2-amino-3-(4-chlorophenyl)propyl]benzo[c]azolidine-1,3-dione hydrochloride (345 mg, 1.1 mmol) to give the title compound (450 mg, 75%). MS (ESI) m/z 544.5 [M+1]⁺.

C. 2-[2-Amino-3-(4-chlorophenyl)propyl]benzo[c]azolidine-1,3-dione hydrochloride. The compound was prepared as in Example 5.1.3, Step C, using (tert-butoxy)-N-{1-[(4-chlorophenyl)methyl]-2-hydroxyethyl}carboxamide (3.82 g, 13.37 mmol) to give the title compound as a white solid (690 mg, 13%). MS (ESI) m/z 315 [M+1]⁺.

D. (Tert-butoxy)-N-{1-[(4-chlorophenyl)methyl]-2-hydroxyethyl}carboxamide. The compound was prepared as in Example 5.1.3, Step D, using (2R)-2-[(tert-butoxy)carbonylamino]-3-(4-chlorophenyl)propanoic acid (5 g, 33.36 mmol) and (2S)-2-[(tert-butoxy)carbonylamino]-3-(4-chlorophenyl)propanoic acid (5 g, 33.36 mmol) to give the title compound (3.83 g, 40%). MS (ESI) m/z 286.1 [M+1]⁺.

### 5.1.14 SYNTHESIS OF [(1R)-2-AMINO-1-(3-PYRIDYLMETHYL)ETHYL](5-(6-ISOQUINOLYL)(1,3,4-THIADIAZOL-2-YL))AMINE

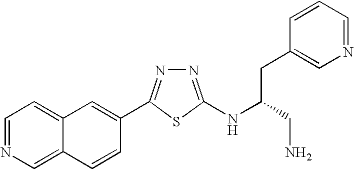

A. [(1R)-2-Amino-1-(3-pyridylmethyl)ethyl](5-(6-isoquinolyl)(1,3,4-thiadiazol-2-yl))amine. N-[({[(1R)-2-(1,3-dioxobenzo[c]azolidin-2-yl)-1-(3-pyridylmethyl)ethyl]amino}thioxomethyl)amino]-6-isoquinolylcarboxamide (190 mg, 0.37 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.0% purity, (70 mg, 52%). ¹H NMR (400 MHz, CD₃OD) δ 9.74 (s, 1H), 8.92 (m, 1H), 8.70 (d, J=5.6 Hz 1H), 8.64 (dt, J=8.4, 1.6 Hz, 1H), 8.36 (m, 5H), 8.00 (dd, J=6.0, 8.4, Hz, 1H), 4.67 (m, J=4.8 Hz, 1H), 3.38 (m, 4H). MS (ESI) m/z 463.4 [M+1]⁺.

B. N-[({[(1R)-2-(1,3-Dioxobenzo[c]azolidin-2-yl)-1-(3-pyridylmethyl)ethyl]amino}thioxomethyl)amino]-6-isoquinolylcarboxamide. (2-((2R)-2-Amino-3-(3-pyridyl)propyl)benzo[c]azolidine-1,3-dione HCl (115 mg, 0.6 mmol) was coupled with isoquinoline-6-carbohydrazide according to General Procedure C. The crude product was precipitated out of ether to give an off-white solid, (190 mg, 61%). MS (ESI) m/z 511.5 [M+1]⁺.

C. (2-((2R)-2-Amino-3-(3-pyridyl)propyl)benzo[c]azolidine-1,3-dione HCl. N-[(1R)-2-Hydroxy-1-(3-pyridylmethyl)ethyl](tert-butoxy)carboxamide (280 mg, 1.1 mmol) was dissolved in THF (10 mL). Then added with triphenylphosphine (320 mg, 1.2 mmol) and phthalimide (180 mg, 1.2 mmol). The reaction was cooled in an ice-bath before addition of diisopropyl azodicarboxylate (247 mg, 1.2 mmol) dropwise via a syringe. The reaction was stirred at room temperature for 16 hours followed by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (214 mg, 55% yield). MS (ESI) m/z 282.3 [M+1]⁺.

D. N-[(1R)-2-Hydroxy-1-(3-pyridylmethyl)ethyl](tert-butoxy)carboxamide. N-Boc-D-3-pyridylalanine (1 g, 3.7 mmol) was added with THF (50 mL) and triethylamine (0.5 mL). The reaction was cooled with an ice-bath and stirred under N₂. Chloroethylformate (358 μL, 3.7 mmol) was added via a syringe and stirred for 30 minutes. Sodium borohydride (428 mg, 11 mmol) was added to the THF solution and then MeOH (25 mL) was dripped into the reaction via an addition funnel over 30 minutes. The reaction was concentrated and then flash chromatographed to give a colorless oil, (280 mg, 29% yield). MS (ESI) m/z 253.4 [M+1]⁺.

### 5.1.15 SYNTHESIS OF 4-(5-{[2-AMINO-1-BENZYLETHYL]AMINO}-1,3,4-THIADIAZOL-2-YL)BENZAMIDE

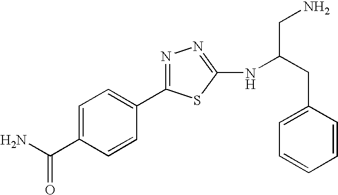

A. 4-(5-{[2-amino-1-benzylethyl]amino}-1,3,4-thiadiazol-2-yl)benzamide. 4-(5-{[2-(1,3-dioxobenzo[c]azolidin-2-yl)-1-benzylethyl]amino}-1,3,4-thiadiazol-2-yl)benzamide (131 mg, 0.27 mmol) was reacted according to General Procedure A. Sample was dissolved in 1:1 DMSO:MeOH and purified using RP-HPLC on a C18 column with 10-70% CH₃CN/H₂O/0.1% TFA to provide the crude title compound (41.3 mg, 43%). ¹H NMR (400 MHz, DMSO) δ 8.08-8.06 (m, 3H), 7.95 (d, J=7.0 Hz), 2H), 7.88 (dd, J=3.0 and 6.0 Hz), 2H); 8.63 (d, J=8.0 Hz, 1H), 7.81 (d, J=8.8 Hz), 2H), 8.34 (s, 1H), 8.07 (m, 1H), 7.36 (m, 2H), 7.29 (m, 2H), 4.37 (m, 1H), 3.26 (dd, J=5.8 and 13.8 Hz, 1H), 3.13 (dd, J=8.0 and 13.8 Hz), 1H); ES-MS (m/z) 354[M+1]⁺.

B. 4-(5-{[2-(1,3-dioxobenzo[c]azolidin-2-yl)-1-benzylethyl]amino}-1,3,4-thiadiazol-2-yl)benzamide. Crude 4-{N-[({[2-(1,3-dioxobenzo[c]azolidine)-1-benzylethyl]amino}thiomethyl)amino]carbamoyl}benzamide (373 mg, 0.74 mmol) was reacted according to General Procedure E and purified using flash chromatography, 98:2 DCM:MeOH afforded the crude title compound (131 mg, 36%). ES-MS (m/z) 484[M+1]⁺

C. 4-{N-[({[2-(1,3-dioxobenzo[c]azolidine)-1-benzylethyl]amino}thiomethyl)amino]carbamoyl}benzamide. 2-(2-amino-3-phenylpropyl)benzo[c]azolidine-1,3-dione (336 mg, 1.2 mmol) and 4-(carbohyrazide) benzamide (226 mg, 1.26 mmol) were reacted according to General Procedure C and purified using flash chromatography, 98:2 DCM:MeOH afforded the crude title compound (373 mg, 62%). ES-MS (m/z) 502[M+1]⁺.

D. 4-(carbohyrazide) benzamide A solution of methyl 4-carbamoylbenzoate (300 mg, 1.67 mmol), hydrazine (2 mL, excess) in EtOH were reacted in a microwave reactor at 130° C. for 30 min. Reaction is concentrated on a rotoevaporator and the product was recrystallized from hot ethanol to afford title compound (225 mg, 75%). ES-MS (m/z) 180[M+1]⁺

### 5.1.16 SYNTHESIS OF ((4S/R,3R/S)-4-PHENYLPYRROLIDIN-3-YL)(5-(6-ISOQUINOLYL)(1,3,4-THIADIAZOL-2-YL))AMINE

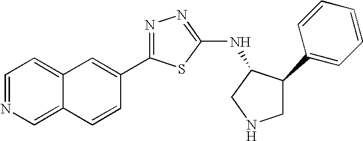

A. ((4S/R,3R/S)-4-phenylpyrrolidin-3-yl)(5-(6-isoquinolyl)(1,3,4-thiadiazol-2-yl))amine. A solution of tert-butyl 3-({[(6-isoquinolylcarbonylamino) amino]thioxomethyl}amino)-4-phenylpyrrolidinecarboxylate (0.27 g, 0.3 mmol) in CH₂Cl₂ (10 mL) and methanol (10 mL) was treated with HCl in dioxane (3 mL, 4M). The resulting reaction was stirred at room temperature overnight. Excess solvents were removed under reduced pressure and the resulting residue (235 mg) was taken up in methane sulfonic acid (1 mL) and stirred at room temperature overnight. The reaction was diluted with water, the resulting acidic solution was neutralized with ammonium hydroxide, and product was extracted with EtOAc. Organic fractions were combined and washed with water and saturated sodium chloride, dried over magnesium sulfate, filtered and solvent removed under reduced pressure to afford crude product. Product was purified using reverse-phase semi-preparatory HPLC (10-40% acetonitrile+0.1% TFA in H₂O+0.1% TFA, over 30 min). Fractions containing clean product were neutralized with potassium carbonate and solvent removed under reduced pressure. The resulting material was taken up in EtOAc, washed with saturated potassium carbonate, water, and saturated sodium chloride in succession, dried over magnesium sulfate, filtered and solvent removed under reduced pressure to afford the title compound, 98.2% purity, (20 mg, 10%). ¹H NMR (300 MHz, CD₃OD) δ 9.26 (s, 1H), 8.48 (d, J=5.7, 1H), 8.22-8.17 (overlapping m, 3H), 7.89 (d, J=5.7, 1H), 7.41-7.24 (overlapping m, 5H), 4.42 (dt, J=7.2, 6.9), 3.62 (dd, J=11.7, 7.2, 1H), 3.51 (dd, J=10.95, 7.95, 1H), 3.36 (m, 1H), 3.07 (dd, J=1.1, 9.0, 1H), 3.02 (dd, J=11.7, 6.0, 1H); MS (ESI) m/z 374.0 [M+1]⁺.

B. (tert-butyl 3-({[(6-isoquinolylcarbonylamino)amino]thioxomethyl}amino)-4-phenyl pyrrolidinecarboxylate. A solution of tert-butyl 3-amino-4-phenylpyrrolidinecarboxylate (0.2 g, 0.76 mmol) in THF (6 mL) was added drop wise to a stirred solution of di(2-pyridyloxy)methane-1-thione (0.18 g, 0.77 mmol) in THF (5 mL). The reaction mixture was stirred 1 hour at room temperature and then solvent removed under reduced pressure. The resulting residue was taken up in ethanol (10 mL) and isoquinoline-6-carbohydrazide (0.14 g, 0.75 mmol) was added and reaction was heated on a 60° C. oil bath overnight. Upon cooling, the product formed as a white precipitate. Material was filtered to provide the title compound, pure, as a white solid (0.278 g, 78%). MS (ESI) m/z 492.1 [M+1]⁺.

### 5.1.17 SYNTHESIS OF {2-AMINO-1-[(3-METHOXYPHENYL) METHYL]ETHYL}(5-(6-ISOQUINOLYL)(1,3,4-THIADIAZOL-2-YL))AMINE

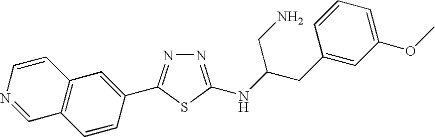

A. {2-amino-1-[(3-methoxyphenyl)methyl]ethyl}(5-(6-isoquinolyl)(1,3,4-thiadiazol-2-yl))amine. A solution of 2-{2-[(5-(6-isoquinolyl)(1,3,4-thiadiazol-2-yl))amino]-3-(3-methoxyphenyl)propyl}benzo[c]azoline-1,3-dione (0.19 g, 0.36 mmol) and hydrazine (0.1 mL, excess) in ethanol (10 mL) was reacted according to General Procedure A. Product was purified using reverse-phase semi-preparatory HPLC (10-40% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.3% purity, (51 mg, 36%). ¹H NMR (300 MHz, CD₃OD) δ 9.25 (s, 1H), 8.48 (d, J=6.04, 1H), 8.20-8.17 (overlapping m, 3H), 7.88 (d, J=5.77, 1H), 7.17 (t, J=8.10, 1H), 6.87 (m, 2H), 6.75 (m, 1H), 4.07 (m, 1H), 3.76 (s, 3H), 2.94 (ABX, J_{AB}=13.74, J_{AX}=6.04, J_{BX}=7.69, 2H), 2.85 (ABX, J_{AB}=13.46, J_{AX}=4.94, J_{BX}=7.69, 2H); MS (ESI) m/z 392.0 [M+1]⁺.

B. 2-{2-[(5-(6-isoquinolyl)(1,3,4-thiadiazol-2-yl))amino]-3-(3-methoxyphenyl)propyl}benzo[c]azoline-1,3-dione. Methane sulfonic acid (2.0 mL, excess) and N-{[({2-(1,3-dioxobenzo[c]azolin-2-yl)-1-[(3-methoxyphenyl)methyl]ethyl}amino)thioxomethyl]amino}-6-isoquinolylcarboxamide (0.235 g, 0.4 mmol) were reacted as described in General Procedure B. Purification using Biotage column chromatography (0-20% methanol in CH₂Cl₂) provided clean product (0.19 g, 83%). MS (ESI) m/z 522.3 [M+1]⁺.

C. N-{[({2-(1,3-dioxobenzo[c]azolin-2-yl)-1-[(3-methoxyphenyl)methyl]ethyl}amino)thioxo methyl]amino}-6-isoquinolylcarboxamide. The HCl salt of 2-[2-amino-3-(3-methoxyphenyl)propyl]benzo[c]azoline-1,3-dione (0.35 g, 1.0 mmol), triethylamine (0.3 mL, 2.1 mmol), di(2-pyridyloxy)methane-1-thione (0.24 g, 1.0 mmol), and isoquinoline-6-carbohydrazide (0.19 g, 1.0 mmol) were reacted according to General Procedure C. Purification using Biotage column chromatography (10-20% methanol in CH₂Cl₂) provided product ˜95% purity (0.25 g, 46%). MS (ESI) m/z 540.5 [M+1]⁺.

D. 2-[2-amino-3-(3-methoxyphenyl)propyl]benzo[c]azoline-1,3-dione hydrochloride. (Tert-butoxy)-N-{2-(1,3-dioxobenzo[c]azolin-2-yl)-1-[(3-methoxyphenyl)methyl]ethyl}carboxamide (0.16 g, 0.4 mmol) and HCl (5 mL, 4M in dioxane, 20.0 mmol) in CH₂Cl₂ (5 mL) and methanol (10 mL) were reacted according to General Procedure D. The resulting white solid was dried under vacuum overnight to give clean product as an HCl salt (0.16 g, 100%). MS (ESI) m/z 311.0 [free base M+1]⁺.

E. (tert-butoxy)-N-{2-(1,3-dioxobenzo[c]azolin-2-yl)-1-[(3-methoxyphenyl)methyl]ethyl}carboxamide. (Tert-butoxy)-N-{2-hydroxy-1-[(3-methoxyphenyl)methyl]ethyl}carboxamide (0.45 g, 1.6 mmol), resin-bound triphenyl phosphine (0.8 g, 3 mmol P/g resin), phthalimide (0.32 g, 2.4 mmol) and diisopropy-azo-dicarboxylate (0.47 mL, 2.4 mmol) were reacted according to General Procedure E. Purification using silica gel flash column chromatography (0-50% EtOAc in hexanes provided the title compound (0.18 g, 27%). MS (ESI) m/z 411.1 [M+1]⁺.

F. (tert-butoxy)-N-{2-hydroxy-1-[(3-methoxyphenyl)methyl]ethyl}carboxamide. Methyl 2-[(tert-butoxy)carbonylamino]-3-(3-methoxyphenyl) propanoate (1.0 g, 3.2 mmol) and lithium aluminum hydride (0.37 g, 9.7 mmol) were reacted at room temperature overnight, as described in General Procedure F. Purification using Biotage column chromatography (10-70% EtOAc in hexanes) provided the title compound (0.5 g, 56%). MS (ESI) m/z 282.3 [M+1]⁺.

G. Methyl 2-[(tert-butoxy)carbonylamino]-3-(3-methoxyphenyl)propanoate. A solution of 2-[(tert-butoxy)carbonylamino]-3-(3-hydroxyphenyl)propanoic acid (1.0 g, 3.6 mmol), lithium hydroxide monohydrate (0.6 g, 14.6 mmol), and dimethyl sulfate (1.0 mL, 10.6 mmol) in THF (25 mL) was stirred overnight at room temperature, followed by a second addition of dimethyl sulfate (1.0 mL) and stirred a further 6 hours. Upon complete conversion of starting material, the reaction was poured into EtOAc, washed with water and saturated sodium chloride in succession, dried over magnesium sulfate, filtered and solvent removed under reduced pressure to afford crude material. Purification using Biotage column chromatography (5-50% EtOAc in hexanes) provided the title compound, after drying under high vacuum overnight, as a colorless oil (1.1 g, 100%). MS (ESI) m/z 310.1 [M+1]⁺.

### 5.1.18 SYNTHESIS OF [2-AMINO-1-BENZYLETHYL](5-(6-ISOQUINOLYLY)(1,3,4-THIADIAZOL-2-YL))AMINE N-OXIDE

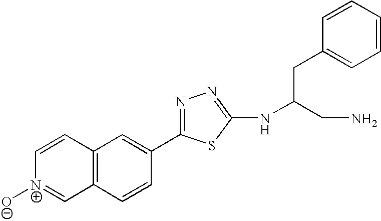

A. [2-Amino-1-benzylethyl](5-(6-isoquinolyl)(1,3,4-thiadiazol-2-yl))amine N-oxide. A solution of N-[({[2-(1,3-dioxobenzo[c]azolin-2-yl)-1-benzylethyl]amino}thioxomethyl)amino]-6-isoquinolylcarboxamide N-oxide (260 mg, 0.450 mmol) in methane sulfonic acid (2 mL) was stirred 1 hour at 60° C. The reaction was cooled, diluted with water, neutralized with saturated aqueous sodium bicarbonate, and then extracted five times with dichloromethane. The organic solution was dried over anhydrous sodium sulfate, filtered, and volatiles evaporated. The material was dried under vacuum, dissolved in DMF (2 mL), and hydrazine monohydrate (2 mL) was added. The reaction was stirred at room temperature for 1 hour and then the volatiles were evaporated. The residue was purified using chromatography on a normal phase silica gel column with 2 to 50% methanol in dichloromethane. Fractions with product were combined and the volatiles were evaporated. The material was further purified on preparatory TLC plates (250 μm, 20×20 cm). The plates were eluted with 15% methanol in dichloromethane and the product was recovered from the silica by extraction with 15% ethyl acetate in hexanes. The solvent was evaporated, the resulting solids were dissolved in 5% ethyl acetate in hexanes, and filtered through a 0.45 μm syringe filter. The material was dried under vacuum at 60° C. to give the title compound (28 mg, 16%, 2 steps). ¹H NMR (400 MHz, DMSO) δ 8.96 (s, 1H), 8.25 (s, 1H), 8.19 (d, J=7.2, 1H), 8.10 (d, J=8.8, 1H), 8.02 (d, J=7.2, 1H), 7.93 (d, J=8.8, 1H), 7.26-7.33 (m, 4H), 7.17-7.22 (m, 1H), 3.93-3.95 (m, 1H), 2.85-2.98 (m, 2H), 2.74-2.85 (m, 2H); MS (ESI) m/z 378.5 [M+1]⁺.

B. N-[({2-(1,3-Dioxobenzo[c]azolin-2-yl)-1-benzylethyl]amino}thioxomethyl)amino]-6-isoquinolylcarboxamide N-oxide. To a solution of 2-(2-amino-3-phenylpropyl)benzo[c]azoline-1,3-dione hydrochloride (297 mg, 1.06 mmol) in dichloromethane (5 mL) was added aqueous sodium carbonate until the mixture was basic to pH paper. The organic phase was separated and the aqueous solution was extracted three times with dichloromethane. The combined organic phases were washed with brine, dried over anhydrous sodium sulfate, and filtered. The solvent was evaporated and the material was dried under vacuum at room temperature. To a solution of the residue in anhydrous THF (4 mL) was added a solution of di-2-pyridyl thionocarbonate (257 mg, 1.11 mmol) in anhydrous THF (8 mL), and anhydrous triethylamine (0.15 mL, 1.06 mmol). The reaction was stirred 1 hour at room temperature and the volatiles were evaporated. To a solution of the resulting residue in anhydrous ethanol (12 mL) was added isoquinoline-6-carbohydrazide N-oxide (225 mg, 1.11 mmol) and the mixture heated to 60° C. with stirring for 90 minutes. The reaction mixture was cooled to room temperature and the resulting precipitate was filtered and rinsed with ethyl acetate. The material was dried under vacuum to give the title compound (260 mg, 46%). MS (ESI) m/z 525.8 [M+1]⁺.

C. Isoquinoline-6-carbohydrazide N-oxide. A solution of isoquinoline-6-carbonitrile N-oxide (1.0 g, 5.88 mmol) in anhydrous methanol (15 mL) was heated to 60° C. and acetyl chloride (8 mL) was added drop wise. The reaction was stirred at 60° C. for 90 minutes and then at room temperature for 90 minutes. To the reaction was added acetyl chloride (12 mL) drop wise in portions over 2 hours and the mixture stirred 18 hours at room temperature. The reaction was diluted with aqueous sodium bicarbonate, extracted 3 times with 5% methanol in dichloromethane, and two times with THF. The solvents were evaporated and the residue purified using reverse-phase preparatory HPLC (10-50% acetonitrile/water/0.1% TFA, over 30 min). Fractions containing clean product were combined and the solution made basic with aqueous ammonium hydroxide. The acetonitrile was evaporated, the solution was saturated with sodium chloride, extracted three times with dichloromethane, and two times with THF. The combined organic phases were dried over anhydrous sodium sulfate, filtered, and volatiles evaporated. The resulting intermediate was stirred with anhydrous methanol (3 mL) and then anhydrous hydrazine (0.1 mL, excess) was added and the mixture heated to 60° C. with stirring for 90 minutes. The reaction mixture was cooled to room temperature and the volatiles evaporated. The material was dried under vacuum to give the title compound (228 mg, 19%). MS (ESI) m/z 204.1 [M+1]⁺.

D. Isoquinoline-6-carbonitrile N-oxide. The title compound was prepared as described in Example 5.1.8, Step D, using 6-bromoisoquinoline N-oxide (5.0 g, 22.32 mmol) to give the title compound (2.41 g, 63%). MS (ESI) m/z 171.3 [M+1]⁺.

E. 6-Bromoisoquinoline N-oxide. A solution of 6-bromoisoquinoline (10.0 g, 48.08 mmol) in anhydrous dichloromethane (475 mL), was chilled in a ice water bath and then 3-chloroperoxybenzoic acid (14.0 g, 62.5 mmol) was added in portions with stirring. The mixture was stirred at room temperature for 18 hours. The reaction was diluted with dichloromethane, washed with aqueous sodium bicarbonate, 10% aqueous sodium thiosulfate, and with brine. The organic solution was dried over anhydrous sodium sulfate, filtered, and volatiles evaporated to give a solid. Impure crystals of product that formed from the combined aqueous washes were filtered and dried. The recovered solids were purified using chromatography on a normal phase silica gel column with 50 to 100% ethyl acetate in hexanes followed by 5 to 15% methanol in dichloromethane. Fractions with pure product were combined and the volatiles were evaporated. The material was dried under vacuum to give the title compound (10.1 g, 93%). MS (ESI) m/z 224.3 [M+1]⁺.

### 5.1.19 SYNTHESIS OF 2-{(2R)-3-(4-FLUOROPHENYL)-2-[(5-(6-ISOQUINOLYL)(1,3,4-THIADIAZOL-2-YL))AMINO]PROPYL}BENZO[c]AZOLIDINE-1,3-DIONE

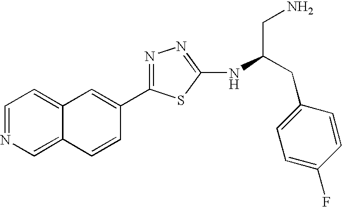

A. 2-{(2R)-3-(4-fluorophenyl)-2-[(5-(6-isoquinolyl)(1,3,4-thiadiazol-2-yl))amino]propyl}benzo[c]azolidine-1,3-dione N-{[({(1R)-2-(1,3-dioxobenzo[c]azolidin-2-yl)-1-[(4-fluoro phenyl)methyl]ethyl}amino)thioxomethyl]amino}-6-isoquinolylcarboxamide was reacted according to General Procedure A. Crystallization from EtOH/Hexane afforded the title compound, (86 mg, 49%). ¹H NMR (400 MHz, CD₃OD) δ 9.16 (s, 1H), 8.39 (d, J=5.6 Hz), 1H), 8.12 (s, 1H); 8.08 (s, 2H), 7.80 (d, J=6.0 Hz), 1H), 7.22-7.19 (m, 2H), 6.90 (t, J=8.8),2H), 3.95 (m, 1H), 2.91 (dd, J=6.4 and 14.0 Hz), 1H); 2.81 (dd, J=7.6 and 14.0 Hz), 2H); 2.69 (s, 1H); ES-MS (m/z) 380[M+1]⁺.

B. N-{[({(1R)-2-(1,3-dioxobenzo[c]azolidin-2-yl)-1-[(4-fluorophenyl)methyl]ethyl}amino)thioxomethyl]amino}-6-isoquinolylcarboxamide. 2-[(2R)-2-amino-3-(4-fluorophenyl)propyl]benzo[c]azolidine-1,3-dione (356 mg, 1.19 mmol) was reacted according to General Procedure C to afford the crude title compound (1.04 g). Crude material was used for next step. ES-MS (m/z) 528[M+1]⁺.

C. 2-[(2R)-2-amino-3-(4-fluorophenyl)propyl]benzo[c]azolidine-1,3-dione. N-{(1R)-2-(1,3-dioxobenzo[c]azolidin-2-yl)-1-[(4-fluorophenyl)methyl]ethyl}(tert-butoxy)carboxamide (388 mg, 0.974 mmol) was reacted according to General Procedure D to afford the title compound as the HCl salt (356 mg, >100%). ES-MS (m/z) 299[M+1]⁺.

D. N-{(1R)-2-(1,3-dioxobenzo[c]azolidin-2-yl)-1-[(4-fluorophenyl)methyl]ethyl}(tert-butoxy)carboxamide N-{(1R)-1-[(4-fluorophenyl)methyl]-2-hydroxyethyl}(tert-butoxy) carboxamide (850 mg, 3.15 mmol) was reacted according to General Procedure E. Purification using flash chromatography, 98:2 DCM:MeOH afforded the crude title compound (388 mg, 30%). ES-MS (m/z) 399[M+1]⁺.

E. N-{(1R)-1-[(4-fluorophenyl)methyl]-2-hydroxyethyl}(tert-butoxy)carboxamide (2R)-2-[(tert-butoxy)carbonylamino]-3-(4-fluorophenyl)propanoic acid (2.0 g, 7.06 mmol) was reacted according to General Procedure E. Purification using flash chromatography, 98:2 DCM:MeOH afforded the crude title compound (850 mg, 45%). ES-MS (m/z) 270[M+1]⁺.

### 5.1.20 SYNTHESIS OF [(1R)-2-AMINO-1-BENZYLETHYL1][5-(3-METHYL(1H-INDAZOL-5-YL))(1,3,4-THIADIAZOL-2-YL)]AMINE

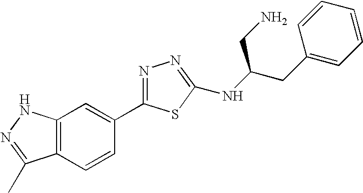

A. [(1R)-2-amino-1-benzylethyl][5-(3-methyl(1H-indazol-5-yl))(1,3,4-thiadiazol-2-yl)]amine. 2-((2R)-2-{[5-(3-methyl(1H-indazol-5-yl))(1,3,4-thiadiazol-2-yl)]amino}-3-phenyl propyl)benzo[c]azoline-1,3-dione (0.680 g., 1.37 mmol) and hydrazine monohydrate were reacted according to General Procedure A and purified via silica gel chromatography (10-20% methanol/methylene chloride) to afford the title compound (0.210 g., 42%). ¹H NMR (400 MHz, CD₃OD) δ 8.05 (q, 1H), 7.87 (d, J=1.59, 8.79, 1H), 7.56 (d, J=0.79, 8.79, 1H), 7.32 (m, 4H), 7.22 (m, 1H), 4.06 (m, 1H), 2.98 (t, 3H), 2.95 (m, 3H), 2.81 (dd, 1H), 2.61 (s, 3H). MS (ESI) m/z 365 [M+1]⁺. Anal. calculated for C₁₉H₂₀N₆S, C, 62.61; H, 5.53; N, 23.06. Found, C, 61.91; H, 5.43; N, 22.91.

B. 2-((2R)-2-{[5-(3-methyl(1H-indazol-5-yl))(1,3,4-thiadiazol-2-yl)]amino}-3-phenylpropyl)benzo[c]azoline-1,3-dione. N-[({[(1R)-2-(1,3-dioxobenzo[c]azolin-2-yl)-1-benzylethyl]amino}thioxomethyl)amino](3-methyl(1H-indazol-5-yl))carboxamide (1.05 g., 2.05 mmol) and methanesulfonic acid (20 mL) were reacted according to General Procedure B and purified via silica gel chromatography (5-7% methanol/methylene chloride) to afford the title compound (0.680 g., 67%). MS (ESI) m/z 495 [M+1]⁺.

C. N-[({[(1R)-2-(1,3-dioxobenzo[c]azolin-2-yl)-1-benzylethyl]amino}thioxomethyl)amino](3-methyl(1H-indazol-5-yl))carboxamide. 2-((2R)-2-amino-3-phenylpropyl)benzo[c]azoline-1,3-dione (0.737 g., 2.63 mmol) and 3-methyl-1H-indazole-5-carbohydrazide (0.500 g., 2.63 mmol) were reacted according to General Procedure C and purified via silica gel chromatography (80-100% ethyl acetate/hexanes) to afford the title compound (1.05 g., 78%). MS (ESI) m/z 513 [M+1]⁺.

D. 3-Methyl-1H-indazole-5-carbohydrazide. Methyl 1-[(tert-butyl)oxycarbonyl]-3-methyl-1H-indazole-5-carboxylate (0.685 g., 2.36 mmol) and hydrazine (0.151 g., 4.72 mmol) were reacted according to General Procedure H to afford the title compound (0.469 g., 100%). MS (ESI) m/z 191 [M+1]⁺.

E. Methyl 1-[(tert-butyl)oxycarbonyl]-3-methyl-1H-indazole-5-carboxylate. To a solution of methyl 3-methyl-1H-indazole-5-carboxylate (1.31 g., 6.89 mmol) and triethylamine (1.39 g., 13.78 mmol) in methylene chloride was added di-tert-butyl dicarbonate (1.73 g., 7.92 mmol). The solution was allowed to stir at ambient conditions for 15 minutes. Additional di-tert-butyl dicarbonate (1.73 g., 7.92 mmol) and triethylamine (0.900 mL) were added to assure complete consumption of starting material via thin layer chromatography (40% ethyl acetate/hexanes). The solution was then condensed under reduced pressure and partitioned between water and ethyl acetate (3×). The organics were dried over magnesium sulfate, filtered and solvent removed under reduced pressure. The resultant solid was purified via silica gel chromatography (40% ethyl acetate/hexanes) to afford a mixture of N1 and N2-Boc protected indazole (1.7 g., 85%). MS (ESI) m/z 291 [M+1]⁺.

F. Methyl 3-methyl-1H-indazole-5-carboxylate. To a solution of a mixture of tert-butyl 5-cyano-3-methyl-1H-indazolecarboxylate (0.500 g., 1.94 mmol) and 3-methyl-1H-indazole-5-carbonitrile (0.487 g., 3.1 mmol) in methanol (20 mL) was added acetyl chloride (15 mL). The solution was allowed to heat to 60° C. for 16 hours. The solution was then condensed under reduced pressure and added to a screw capped flask with conc. HCl (10 mL) and heated to 100° C. The resultant carboxylic acid was then condensed under reduced pressure and diluted with ethanol (50 mL) and sulfuric acid (1 mL). The solution was heated to 85° C. for 16 hours. The resultant solution was condensed under reduced pressure and partitioned between 1.5M K₂CO₃ and ethyl acetate (3×). The organics were dried over magnesium sulfate, filtered and solvent removed under reduced pressure to afford the title compound as a mixture of methyl and ethyl esters (1.31 g., >100%). MS (ESI) m/z 191 [M+1]⁺ and 205 [M+1]⁺ for the methyl and ethyl ester respectively.

G. tert-butyl 5-cyano-3-methyl-1H-indazolecarboxylate and 3-methyl-1H-indazole-5-carbonitrile. A solution of tert-butyl 5-bromo-3-methyl-1H-indazolecarboxylate (1.87 g., 6.01 mmol), zinc cyanide (1.40 g., 12.02 mmol) and tetrakis(triphenylphosphine)palladium(0) in DMF was heated to 95° C. for 16 hours under a nitrogen atmosphere. After complete consumption of starting material, the solution was filtered through celite and washed with additional ethyl acetate. The filtrant was condensed under reduced pressure and the resultant oil partitioned between water and ethyl acetate (3×). The organics were dried over magnesium sulfate, filtered and solvent removed under reduced pressure to afford a yellow solid. The title compounds (0.500 g. and 0.485 g.) Boc protected and des-Boc protected respectively were isolated after purification via silica gel chromatography (20-35% ethyl acetate/hexanes). MS (ESI) m/z 258 [M+1]⁺ and 158 [M+1]⁺ for the N1-boc protected and free indazole respectively.

H. tert-butyl 5-bromo-3-methyl-1H-indazolecarboxylate. 1-(5-bromo-2-fluorophenyl)ethan-1-one (6.7 g., 30.87 mmol) was dissolved in hydrazine (25 mL) and allowed to stir at 110° C. for 16 hours in a screw capped flask. The solution was cooled and poured into ice water. The resultant precipitate was filtered and dried to afford the title compound (5.50 g., 84%). MS (ESI) m/z 210 [M+1]⁺, 212 [M+2]⁺.

I. 1-(5-bromo-2-fluorophenyl)ethan-1-one. To a solution of 1-(5-bromo-2-fluoro phenyl)ethan-1-ol (8.66 g., 39.54 mmol) in 1,4-dioxane (175 mL)was added MnO₂ (20.0 g., 197.7 mmol). The solution was heated to 115° C. under a nitrogen atmosphere for 16 hours. The solution was filtered through celite and condensed under reduced pressure to afford the title compound (7.1 g., 82%). MS (ESI) m/z 216 [M+1]⁺, 218 [M+2]⁺.

J. 1-(5-bromo-2-fluorophenyl)ethan-1-ol. To a solution of 5-Bromo-2-fluorobenzaldehyde (10.0 g., 49.26 mmol) in anhydrous ether (60 mL) at 0° C. was added 1.4M methyl magnesium bromide (THF/Tol. solution) (36.9 mL) dropwise. The solution stirred for 30 minutes and quenched with water (50 mL) followed by 1N HCl (10 mL). The organics were separated and dried over magnesium sulfate, filtered and solvent removed under reduced pressure to afford the title compound (10.8 g., 100%). MS (ESI) m/z 218 [M+1]⁺, 220 [M+2]⁺.

### 5.1.21 SYNTHESIS OF [(1S)-2-AMINO-1-BENZYLETHYL][5-(3-METHYL(1H-INDAZOL-5-YL))(1,3,4-THIADIAZOL-2-YL)]AMINE

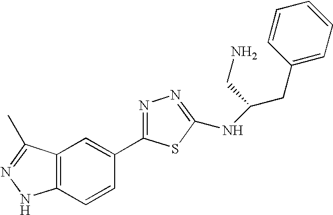

A. [(1S)-2-amino-1-benzylethyl][5-(3-methyl(1H-indazol-5-yl))(1,3,4-thiadiazol-2-yl)]amine. 2-((2S)-2-{[5-(3-methyl(1H-indazol-5-yl))(1,3,4-thiadiazol-2-yl)]amino}-3-phenylpropyl)benzo[c]azoline-1,3-dione (0.248 g., 0.502 mmol) and hydrazine monohydrate (0.4 mL) were reacted according to General Procedure A and purified via silica gel chromatography (10-20% methanol/methylene chloride) to afford the title compound (0.100 g., 55%%). ¹H NMR (400 MHz, CD₃OD) δ 8.05 (s, 1H), 7.87 (d, J=8.79, 1H), 7.56 (d, J=9.59, 1H), 7.31 (m, 4H), 7.21 (m, 1H), 4.06 (m, 1H), 2.98 (t, 3H), 2.95 (m, 3H), 2.82 (dd, 1H), 2.61 (s, 3H). MS (ESI) m/z 365 [M+1]⁺. Anal. calculated for C₁₉H₂₀N₆S, C, 62.61; H, 5.53; N, 23.06. Found, C, 62.16; H, 5.15; N, 22.59.

B. 2-((2S)-2-{[5-(3-methyl(1H-indazol-5-yl))(1,3,4-thiadiazol-2-yl)]amino}-3-phenylpropyl)benzo[c]azoline-1,3-dione. N-[({[(1S)-2-(1,3-dioxobenzo[c]azolin-2-yl)-1-benzylethyl]amino}thioxomethyl)amino](3-methyl(1H-indazol-5-yl))carboxamide (0.305 g., 0.595 mmol) and methanesulfonic acid (8 mL) were reacted according to General Procedure B to afford the title compound (0.248 g., 84%). MS (ESI) m/z 495 [M+1]⁺.

C. N-[({[(1S)-2-(1,3-dioxobenzo[c]azolin-2-yl)-1-benzylethyl]amino}thioxomethyl)amino] (3-methyl(1H-indazol-5-yl))carboxamide. 2-((2S)-2-amino-3-phenylpropyl)benzo[c]azoline-1,3-dione (0.436 g., 1.38 mmol) and 3-methyl-1H-indazole-5-carbohydrazide (0.250 g., 1.31 mmol) were reacted according to General Procedure C and purified via silica gel chromatography (85-100% ethyl acetate/hexanes) to afford the title compound (0.305 g., 82%). MS (ESI) m/z 513 [M+1]⁺.

### 5.1.22 SYNTHESIS OF [(1S)-2-AMINO-1-(2-PYRIDYLMETHYL)ETHYL](5-(6-ISOQUINOLYL)(1,3,4-THIADIAZOL-2-YL))AMINE

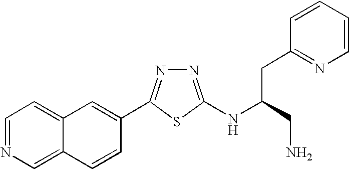

A. [(1S)-2-Amino-1-(2-pyridylmethyl)ethyl](5-(6-isoquinolyl)(1,3,4-thiadiazol-2-yl))amine. N-[({[(1S)-2-(1,3-Dioxobenzo[c]azolidin-2-yl)-1-(2-pyridylmethyl)ethyl]amino}thioxomethyl)amino]-6-isoquinolylcarboxamide (270 mg, 0.53 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.1% purity, (48 mg, 25%). ¹H NMR (400 MHz, CD₃OD) δ 9.22 (s, 1H), 8.44 (m, 2H), 8.17 (s, 1H), 8.13 (s, 2H), 7.85 (d, J=6.0 Hz, 1H), 7.78 (td, J=8.0, 2.0 Hz, 2H), 7.37 (d, J=7.6 Hz, 1H), 7.21 (m, 1H), 4.19 (m, 1H), 3.10 (ddd, J=5.6, 14.0, 36.4 Hz, 2H), 2.84 (ddd, J=5.2, 10.0, 37.2 Hz, 2H). MS (ESI) m/z 363.3 [M+1]⁺.

B. N-[({[(1S)-2-(1,3-Dioxobenzo[c]azolidin-2-yl)-1-(2-pyridylmethyl)ethyl]amino}thioxomethyl)amino]-6-isoquinolylcarboxamide. (2-((2S)-2-Amino-3-(2-pyridyl)propyl)benzo[c]azolidine-1,3-dione HCl (187 mg, 0.5 mmol) was coupled with isoquinoline-6-carbohydrazide according to General Procedure C. The crude product was precipitated out of ether to give an off-white solid. MS (ESI) m/z 511.4 [M+1]⁺.

C. (2-((2S)-2-amino-3-(2-pyridyl)propyl)benzo[c]azolidine-1,3-dione HCl. N-[(1S)-2-hydroxy-1-(2-pyridylmethyl)ethyl](tert-butoxy)carboxamide (0.98 g, 3.9 mmol) was dissolved in THF (30 mL). Then added with triphenylphosphine (1.3 g, 5 mmol) and phthalimide (743 mg, 5 mmol). The reaction was cooled in an ice-bath before diisopropyl azodicarboxylate (1 g, 5 mmol) was added dropwise through a syringe. The reaction was stirred at room temperature for 1 hour. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.66 g, 55% yield). MS (ESI) m/z 282.0 [M+1]⁺.

D. N-[(1S)-2-Hydroxy-1-(2-pyridylmethyl)ethyl](tert-butoxy)carboxamide. N-Boc-L-2-pyridylalanine (1 g, 3.7 mmol) was dissolved in THF (10 mL). Lithium aluminum hydride was added to the THF solution in small portions and then allowed to stirred for 2 hours. The reaction was quenched with saturated aqueous Na₂SO₄ (2 mL), and then triturated in 2-propanol. The 2-propanol mixture was filtered and then concentrated to give a colorless oil, (0.97 g, 99% yield). MS (ESI) m/z 253.1 [M+1]⁺.

### 5.1.23 SYNTHESIS OF [(1R)-2-AMINO-1-(2-PYRIDYLMETHYL)ETHYL](5-(6-ISOQUINOLYL)(1,3,4-THIADIAZOL-2-YL))AMINE

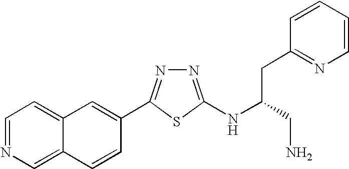

A. [(1R)-2-Amino-1-(2-pyridylmethyl)ethyl](5-(6-isoquinolyl)(1,3,4-thiadiazol-2-yl))amine. N-[({[(1R)-2-(1,3-Dioxobenzo[c]azolidin-2-yl)-1-(2-pyridylmethyl)ethyl]amino}thioxomethyl)amino]-6-isoquinolylcarboxamide (350 mg, 0.71 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.1% purity, (175 mg, 48%). ¹H NMR (400 MHz, CD₃OD) δ 9.21 (s, 1H), 8.44 (m, 2H), 8.31 (d, J=4.8 Hz, 1H), 8.17 (s, 1H), 8.12 (s, 2H), 7.84 (d, J=6.0 Hz, 1H), 7.78 (td, J=8.0, 2.0 Hz, 2H), 7.32 (d, J=7.6 Hz, 1H), 7.21 (m, 1H), 4.19 (m, 1H), 3.10 (ddd, J=5.6, 14.0, 36.4 Hz, 2H), 2.84 (ddd, J=5.2, 10.0, 37.2 Hz, 2H). MS (ESI) m/z 363.3 [M+1]⁺.

B. N-[({[(1R)-2-(1,3-Dioxobenzo[c]azolidin-2-yl)-1-(2-pyridylmethyl)ethyl]amino}thioxomethyl)amino]-6-isoquinolylcarboxamide. (2-((2S)-2-Amino-3-(2-pyridyl)propyl)benzo[c]azolidine-1,3-dione HCl (351 mg, 1 mmol) was coupled with isoquinoline-6-carbohydrazide according to General Procedure C. The crude product was precipitated out of ether to give an off-white solid. MS (ESI) m/z 511.5 [M+1]⁺.

C. (2-((2R)-2-Amino-3-(2-pyridyl)propyl)benzo[c]azolidine-1,3-dione HCl. N-[(1R)-2-Hydroxy-1-(2-pyridylmethyl)ethyl](tert-butoxy)carboxamide (0.98 g, 3.9 mmol) was dissolved in THF (30 mL). Then added with triphenylphosphine (1.3 g, 5 mmol) and phthalimide (743 mg, 5 mmol). The reaction was cooled in an ice-bath before diisopropyl azodicarboxylate (1 g, 5 mmol) was added dropwise through a syringe. The reaction was stirred at room temperature for 1 hour. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.52 g, 47% yield). MS (ESI) m/z 282.4 [M+1]⁺.

D. N-[(1R)-2-Hydroxy-1-(2-pyridylmethyl)ethyl](tert-butoxy)carboxamide. N-Boc-D-2-pyridylalanine (1 g, 3.7 mmol) was dissolved in THF (10 mL). Lithium aluminum hydride (286 mg, 7.5 mmol) was added to the THF solution in small portions and then allowed to stirred for 2 hours. The reaction was quenched with saturated aqueous Na₂SO₄ (2 mL), and then triturated in 2-propanol. The 2-propanol mixture was filtered and then concentrated to give a colorless oil, (0.97 g, 99% yield). MS (ESI) m/z 253.1 [M+1]⁺.

### 5.1.24 SYNTHESIS OF [(1S)-2-AMINO-1-(3-PYRIDYLMETHYL)ETHYL](5-(6-ISOQUINOLYL)(1,3,4-THIADIAZOL-2-YL))AMINE

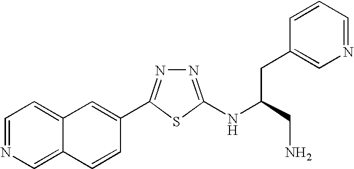

A. [(1S)-2-Amino-1-(3-pyridylmethyl)ethyl](5-(6-isoquinolyl)(1,3,4-thiadiazol-2-yl))amine. N-[({[(1S)-2-(1,3-Dioxobenzo[c]azolidin-2-yl)-1-(3-pyridylmethyl)ethyl]amino}thioxomethyl)amino]-6-isoquinolylcarboxamide (90 mg, 0.25 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent was removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.1% purity, (12 mg, 13%). ¹H NMR (400 MHz, CD₃OD) δ 9.22 (s, 1H), 8.44 (m, 2H), 8.31 (d, J=4.8 Hz, 1H), 8.17 (s, 1H), 8.12 (s, 2H), 7.84 (d, J=6.0 Hz, 1H), 7.78 (dt, J=7.6 Hz, 2H), 7.32 (dd, J=4.8, 8.0 Hz), 4.08 (m, 1H), 3.08 (dd, J=5.2, 18.4 Hz, 1H), 2.88 (m, 2H), 3.08 (dd, J=7.6, 13.6 Hz, 1H). MS (ESI) m/z 463.3 [M+1]⁺.

B. N-[({[(1S)-2-(1,3-Dioxobenzo[c]azolidin-2-yl)-1-(3-pyridylmethyl)ethyl]amino}thioxomethyl)amino]-6-isoquinolylcarboxamide. (2-((2S)-2-Amino-3-(3-pyridyl)propyl)benzo[c]azolidine-1,3-dione HCl (115 mg, 0.6 mmol) was coupled with isoquinoline-6-carbohydrazide according to General Procedure C. The crude product was precipitated out of ether to give an off-white solid. MS (ESI) m/z 511.4 [M+1]⁺.

C. (2-((2S)-2-Amino-3-(3-pyridyl)propyl)benzo[c]azolidine-1,3-dione HCl. N-[(1S)-2-Hydroxy-1-(3-pyridylmethyl)ethyl](tert-butoxy)carboxamide (0.98 g, 3.9 mmol) was dissolved in THF (10 mL). Then added with triphenylphosphine (1.3 g, 5 mmol) and phthalimide (743 mg, 5 mmol). The reaction was cooled in an ice-bath before diisopropyl azodicarboxylate (1 g, 5 mmol) was added dropwise through a syringe. The reaction was stirred at room temperature for 3 hours. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.7 g, 59% yield). MS (ESI) m/z 282.0 [M+1]⁺.

D. N-[(1S)-2-hydroxy-1-(3-pyridylmethyl)ethyl](tert-butoxy)carboxamide. N-Boc-L-3-pyridylalanine (1 g, 3.7 mmol) was dissolved in THF (10 mL). Lithium aluminum hydride was added to the THF solution in small portions and then allowed to stirred for 1 hour. The reaction was quenched with saturated aqueous Na₂SO₄ (2 mL), and then triturated in 2-propanol. The 2-propanol mixture was filtered and then concentrated to give a colorless oil, (0.8 g, 87% yield). MS (ESI) m/z 253.1 [M+1]⁺.

### 5.1.25 SYNTHESIS OF [(1S)-2-AMINO-1-BENZYLETHYL](5-PYRIDINO[3,4-E]PYRIDIN-2-YL(1,3,4-THIADIAZOL-2-YL))AMINE

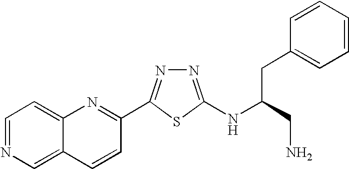

A. [(1S)-2-Amino-1-benzylethyl](5-pyridino[3,4-e]pyridin-2-yl(1,3,4-thiadiazol-2-yl))amine. N-[({[(1S)-2-(1,3-Dioxobenzo[c]azolidin-2-yl)-1-benzylethyl]amino}thioxomethyl)amino]-6-isoquinolylcarboxamide (98 mg, 0.19 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.5% pure, (20 mg, 29%). ¹H NMR (400 MHz, CD₃OD) δ 9.26 (s, 1H), 8.65 (d, J=6.0 Hz, 1H), 8.38 (d, J=8.0 Hz, 1H), 8.33 (d, J=8.8 Hz, 1H), 7.88 (d, J=6.0 Hz, 1H), 7.24 (m, 4H), 7.13 (m, 1H), 4.05 (m, 1H), 2.97 (dd, J=6.0, 13.6 Hz, 1H), 2.88 (m, 2H), 2.74 (dd, J=7.6, 13.6 Hz, 1H). MS (ESI) m/z 363.3 [M+1]⁺.

B. N-[({[(1S)-2-(1,3-Dioxobenzo[c]azolidin-2-yl)-1-benzylethyl]amino}thioxomethyl)amino]-6-isoquinolylcarboxamide. 2-((2S)-2-Amino-3-phenylpropyl)benzo[c]azolidine-1,3-dione HCl (167 mg, 0.5 mmol) and pyridino[4,3-b]pyridine-2-carbohydrazide (100 mg, 0.5 mmol) was coupled according to General Procedure C. The crude product was precipitated out of ether to give an off-white solid, (98 mg, 36% yield). MS (ESI) m/z 511.5 [M+1]⁺.

C. Pyridino[4,3-b]pyridine-2-carbohydrazide. Methyl pyridino[3,4-e]pyridine-2-carboxylate (0.96 g, 5 mmol) was dissolved in EtOH (10 mL) and then added anhydrous hydrazine (2 mL). The reaction was heated in a microwave (130° C., 10 min.) and then concentrated under reduced pressure. The reaction was triturated in ether and then filtered to give an off-white solid, (0.8 g, 83% yield).

D. Methyl pyridino[3,4-e]pyridine-2-carboxylate. Pyridino[4,3-b]pyridine-2-carboxylic acid (1.3 g, 7.5 mmol) was dissolved in MeOH (10 mL) and added with concentrated sulfuric acid (1 mL). The reaction was heated in a microwave (100° C., 10 min.) and then concentrated under reduced pressure. The reaction was then extracted with EtOAc, saturated NaHCO₃, water, and brine. The organic layer was dried over Na₂SO₄, filtered, and concentrated to give a white solid, (0.96 g, 69% yield).

### 5.1.26 SYNTHESIS OF [(1R)-2-AMINO-1-BENZYLETHYL](5-PYRIDINO[3,4-E]PYRIDIN-2-YL(1,3,4-THIADIAZOL-2-YL))AMINE

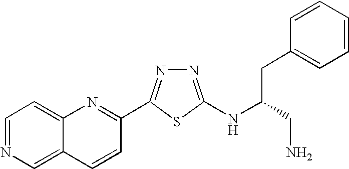

A. [(1R)-2-Amino-1-benzylethyl](5-pyridino[3,4-e]pyridin-2-yl(1,3,4-thiadiazol-2-yl))amine. N-[({[(1R)-2-(1,3-Dioxobenzo[c]azolidin-2-yl) 1-benzylethyl]amino}thioxomethyl)amino]-6-isoquinolylcarboxamide (0.81 g, 1.6 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.5% purity, (467 mg, 81%). ¹H NMR (400 MHz, CD₃OD) δ 9.26 (s, 1H), 8.65 (d, J=6.0 Hz, 1H), 8.38 (d, J=8.0 Hz, 1H), 8.33 (d, J=8.8 Hz, 1H), 7.88 (d, J=6.0 Hz, 1H), 7.24 (m, 4H), 7.13 (m, 1H), 4.05 (m, 1H), 2.97 (dd, J=6.0, 13.6 Hz, 1H), 2.88 (m, 2H), 2.74 (dd, J=7.6, 13.6 Hz, 1H). MS (ESI) m/z 363.4 [M+1]⁺.

B. N-[({[(1R)-2-(1,3-Dioxobenzo[c]azolidin-2-yl)-1-benzylethyl]amino}thioxomethyl)amino]-6-isoquinolylcarboxamide. 2-((2R)-2-Amino-3-phenylpropyl)benzo[c]azolidine-1,3-dione HCl (670 mg, 2 mmol) and pyridino[4,3-b]pyridine-2-carbohydrazide (400 mg, 2 mmol) was coupled according to General Procedure C. The crude product was precipitated out of ether to give an off-white solid, (0.81 g, 74% yield). MS (ESI) m/z 511.3 [M+1]⁺.

### 5.1.27 SYNTHESIS OF [(1R)-2-AMINO-1-(4-PYRIDYLMETHYL)ETHYL](5-(6-ISOQUINOLYL)(1,3,4-THIADIAZOL-2-YL))AMINE

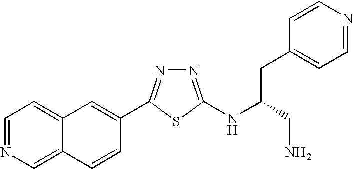

A. [(1R)-2-Amino-1-(4-pyridylmethyl)ethyl](5-(6-isoquinolyl)(1,3,4-thiadiazol-2-yl))amine. N-[({[(1R)-2-(1,3-Dioxobenzo[c]azolidin-2-yl)-1-(4-pyridylmethyl)ethyl]amino}thioxomethyl)amino]-6-isoquinolylcarboxamide (230 mg, 0.45 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 97.6% pure, (13 mg, 8%). ¹H NMR (400 MHz, CD₃OD) δ 9.22 (s, 1H), 8.44 (d, J=6.0 Hz, 1H), 8.38 (d, J=6.4 Hz 2H), 8.18 (s, 1H), 8.14 (s, 2H), 7.85 (d, J=5.2 Hz, 1H), 7.36 (d, J=6.4 Hz, 2H), 4.12 (m, 1H), 3.37 (dd, J=6.0, 13.2 Hz, 1H), 2.83 (m, 3H). MS (ESI) m/z 463.3 [M+1]⁺.

B. N-[({[(1R)-2-(1,3-Dioxobenzo[c]azolidin-2-yl)-1-(4-pyridylmethyl)ethyl]amino}thioxomethyl)amino]-6-isoquinolylcarboxamide. 2-((2R)-2-Amino-3-(4-pyridyl)propyl)benzo[c]azolidine-1,3-dione HCl (100 mg, 0.53 mmol) was coupled with isoquinoline-6-carbohydrazide according to General Procedure C. The crude product was precipitated out of ether to give an off-white solid, (230 mg, 84% yield). MS (ESI) m/z 511.4 [M+1]⁺.

C. 2-((2R)-2-Amino-3-(4-pyridyl)propyl)benzo[c]azolidine-1,3-dione HCl. N-[(1R)-2-Hydroxy-1-(4-pyridylmethyl)ethyl](tert-butoxy)carboxamide (0.98 g, 3.9 mmol) was dissolved in THF (10 mL). Then added with triphenylphosphine (1.3 g, 5-mmol) and phthalimide (743 mg, 5 mmol). The reaction was cooled in an ice-bath before diisopropyl azodicarboxylate (1 g, 5 mmol) was added dropwise through a syringe. The reaction was stirred at room temperature for 3 hours follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.72 g, 60% yield). MS (ESI) m/z 282.0 [M+1]⁺.

D. N-[(1R)-2-Hydroxy-1-(4-pyridylmethyl)ethyl](tert-butoxy)carboxamide. N-Boc-D-4-pyridylalanine (1 g, 3.7 mmol) was dissolved in THF (10 mL). Lithium aluminum hydride was added to the THF solution in small portions and then allowed to stirred for 1 hour. The reaction was quenched with saturated aqueous Na₂SO₄ (2 mL), and then triturated in 2-propanol. The 2-propanol mixture was filtered and then concentrated to give a colorless oil, (0.95 g, 95% yield). MS (ESI) m/z 253.1 [M+1]⁺.

### 5.1.28 SYNTHESIS OF [(1S)-2-AMINO-1-(4-PYRIDYLMETHYL)ETHYL](5-(6-ISOQUINOLYL)(1,3,4-THIADIAZOL-2-YL))AMINE

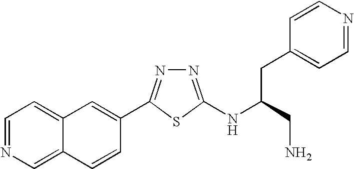

A. [(1S)-2-Amino-1-(4-pyridylmethyl)ethyl](5-(6-isoquinolyl)(1,3,4-thiadiazol-2-yl))amine. N-[({[(1S)-2-(1,3-Dioxobenzo[c]azolidin-2-yl)-1-(4-pyridylmethyl)ethyl]amino}thioxomethyl)amino]-6-isoquinolylcarboxamide (240 mg, 0.47 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.5% purity, (24 mg, 16%). ¹H NMR (400 MHz, CD₃OD) δ 9.22 (s, 1H), 8.44 (d, J=6.0 Hz, 1H), 8.38 (d, J=6.4 Hz 2H), 8.18 (s, 1H), 8.14 (s, 2H), 7.85 (d, J=5.2 Hz, 1H), 7.36 (d, J=6.4 Hz, 2H), 4.12 (m, 1H), 3.37 (dd, J=6.0, 13.2 Hz, 1H), 2.83 (m, 3H). MS (ESI) m/z 363.1 [M+1]⁺.

B. N-[({[(1S)-2-(1,3-Dioxobenzo[c]azolidin-2-yl)-1-(4-pyridylmethyl)ethyl]amino}thioxomethyl)amino]-6-isoquinolylcarboxamide. 2-((2S)-2-Amino-3-(4-pyridyl)propyl)benzo[c]azolidine-1,3-dione HCl (100 mg, 0.53 mmol) was coupled with isoquinoline-6-carbohydrazide according to General Procedure C. The crude product was precipitated out of ether to give an off-white solid, (240 mg, 88% yield). MS (ESI) m/z 511.4 [M+1]⁺.

C. 2-((2S)-2-Amino-3-(4-pyridyl)propyl)benzo[c]azolidine-1,3-dione HCl. N-[(1S)-2-Hydroxy-1-(4-pyridylmethyl)ethyl](tert-butoxy)carboxamide (0.98 g, 3.9 mmol) was dissolved in THF (10 mL). Then added with triphenylphosphine (1.3 g, 5 mmol) and phthalimide (743 mg, 5 mmol). The reaction was cooled in an ice-bath before diisopropyl azodicarboxylate (1 g, 5 mmol) was added dropwise through a syringe. The reaction was stirred at room temperature for 3 hours. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.86 g, 72% yield). MS (ESI) m/z 282.0 [M+1]⁺.

D. N-[(1S)-2-Hydroxy-1-(4-pyridylmethyl)ethyl](tert-butoxy)carboxamide. N-Boc-L-4-pyridylalanine (1 g, 3.7 mmol) was dissolved in THF (10 mL). Lithium aluminum hydride was added to the THF solution in small portions and then allowed to stirred for 1 hour. The reaction was quenched with saturated aqueous Na₂SO₄ (2 mL), and then triturated in 2-propanol. The 2-propanol mixture was filtered and then concentrated to give a colorless oil, (0.95 g, 95% yield). MS (ESI) m/z 253.1 [M+1]⁺.

### 5.1.29 SYNTHESIS OF {2-AMINO-1-[(3-PROPOXYPHENYL)METHYL]ETHYL}(5-(6-ISOQUINOLYL)(1,3,4-THIADIAZOL-2-YL))AMINE

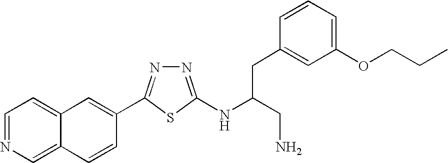

A. {2-Amino-1-[(3-propoxyphenyl)methyl]ethyl}(5-(6-isoquinolyl)(1,3,4-thiadiazol-2-yl))amine. N-{[({2-Amino-1-[(3-propoxyphenyl)methyl]ethyl}amino)thioxomethyl]amino}-6-isoquinolylcarboxamide (340 mg, 0.6 mmol) was cyclized according to General Procedure B to give a yellow solid. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 97.1% purity, (58 mg, 33%). ¹H NMR (400 MHz, CD₃OD) δ 9.22 (s, 1H), 8.44 (d, J=5.6 Hz, 1H), 8.17 (s, 1H), 8.13 (m, 2H), 7.85 (d, J=6.0 Hz, 1H), 7.12 (t, J=7.6 Hz, 1H), 6.81 (m, 3H), 6.68 (d, J=6.0 Hz, 1H), 4.02 (m, J=6.8 Hz, 1H), 3.84 (t, J=6.8 Hz, 2H), 2.88 (m, 3H), 2.74 (dd, J=7.6, 13.6 Hz, 1H), 1.70 (dt, J=7.2 Hz, 2H), 0.94 (t, J=7.2 Hz, 2H). MS (ESI) m/z 420.4 [M+1]⁺.

B. N-{[({2-Amino-1-[(3-propoxyphenyl)methyl]ethyl}amino)thioxomethyl]amino}-6-isoquinolylcarboxamide. (1-Amino-2-phenylethyl) dimethylamine (187 mg, 1 mmol) was coupled with isoquinoline-6-carbohydrazide according to General Procedure C. The crude product was precipitated out of ether to give an off-white solid, (340 mg, 60%). MS (ESI) m/z 568.3 [M+1]⁺.

C. (tert-Butoxy)-N-{2-(1,3-dioxobenzo[c]azolidin-2-yl)-1-[(3-propoxyphenyl)methyl]ethyl}carboxamide. (tert-Butoxy)-N-{2-hydroxy-1-[(3-propoxyphenyl)methyl]ethyl}carboxamide (0.45 g, 1.5 mmol) underwent Mitsunobu reaction condition according to General Procedure E to give the phthalimide. The crude reaction was concentrated under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.41 g, 76% yield). MS (ESI) m/z 339.3 [M+1]⁺.

D. (tert-Butoxy)-N-{2-hydroxy-1-[(3-propoxyphenyl)methyl]ethyl}carboxamide. Propyl 2-[(tert-butoxy)carbonylamino]-3-(3-propoxyphenyl)propanoate (0.55 g, 1.5 mmol) was dissolved in THF (10 mL). Lithium aluminum hydride (60 mg, 1.5 mmol) was added to the THF solution in small portions and then allowed the reaction to stirred for 1 hour. The reaction was quenched with saturated aqueous Na₂SO₄ (2 mL), and then triturated in 2-propanol. The 2-propanol mixture was filtered and then concentrated to give a colorless oil, (0.45 g, 98% yield). MS (ESI) m/z 310.5 [M+1]⁺.

E. Propyl 2-[(tert-butoxy)carbonylamino]-3-(3-propoxyphenyl)propanoate. DL meta-tyrosine was reacted according to General procedure G to give the N-Boc amino acid. 2-[(tert-Butoxy)carbonylamino]-3-(3-hydroxyphenyl)propanoic acid (1.5 g, 5.5 mmol), tetrabutylammonium iodide (50 mg, 0.13 mmol), 1-bromopropane (4 g, 33 mmol), cesium carbonate (2.7 g, 8.3 mmol), and DMF were heated together to 60° C. for 8 hours. Ethyl acetate was added to the reaction and then extracted with water follow with brine. The organic layer was dried over MgSO₄, filtered, and concentrated to give a colorless oil. The oil was flash chromatographed with 10%-20% EtOAc-hexane to give an oil, (0.55 g, 27% yield). MS (ESI) m/z 366.4 [M+1]⁺.

### 5.1.30 SYNTHESIS OF [(1R)-2-AMINO-1-BENZYLETHYL][5-(8-METHOXYPYRIDINO[3,4-e]PYRIDIN-2-YL)(1,3,4-THIADIAZOL-2-YL)]AMINE

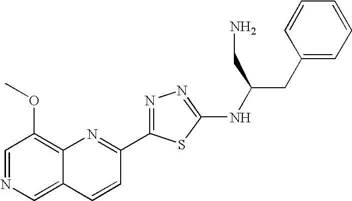

A. [(1R)-2-amino-1-benzylethyl][5-(8-methoxypyridino[3,4-e]pyridin-2-yl)(1,3,4-thiadiazol-2-yl)l amine. 2-((2R)-2-{[5-(8-methoxypyridino[3,4-e]pyridin-2-yl)(1,3,4-thiadiazol-2-yl)]amino}-3-phenylpropyl)benzo[c]azoline-1,3-dione (0.375 g., 0.670 mmol) and hydrazine monohydrate (0.167 g., 3.35 mmol) were reacted according to General Procedure A and purified via preparative HPLC (10-80% acetonitrile/water, 60 mL/min.). Clean fractions were combined and condensed under reduced pressure to give the title compound as the trifluoroacetic acid salt. The solid was dissolved in methanol and 4N HCl/Dioxane was added and the solution condensed under reduced pressure (3×) to give the title compound as the HCl salt (0.155 g., 33%). ¹H NMR (400 MHz, CD₃OD) δ 9.41 (s, 1H), 8.86 (dd, J=2.79, 8.39, 1H), 8.68 (dd, J=2.39, 8.79, 1H), 8.52 (s, 1H), 7.32 (t, 4H), 7.23 (bs, 1H), 4.53 (bm, 1H), 4.27 (s, 3H), 3.08 (m, 2H). MS (ESI) m/z 393 [M+1]⁺.

B. 2-((2R)-2-{[5-(8-methoxypyridino[3,4-e]pyridin-2-yl)(1,3,4-thiadiazol-2-yl)]amino}-3-phenylpropyl)benzo[c]azoline-1,3-dione. N-[({[(1R)-2-(1,3-dioxobenzo[c]azolin-2-yl)-1-benzylethyl]amino}thioxomethyl)amino](8-methoxypyridino[3,4-e]pyridin-2-yl)carboxamide (0.480 g., 0.888 mmol) and methanesulfonic acid (10 mL) were reacted according to General Procedure B to afford the title compound (0.375 g., 81%). MS (ESI) m/z 523 [M+1]⁺.

C. N-[({[(1R)-2-(1,3-dioxobenzo[c]azolin-2-yl)-1-benzylethyl]amino}thioxomethyl)amino](8-methoxypyridino[3,4-e]pyridin-2-yl)carboxamide. 8-methoxypyridino[3,2-c]pyridine-2-carbohydrazide (0.160 g., 0.733 mmol) and 2-((2R)-2-amino-3-phenylpropyl)benzo[c]azoline-1,3-dione:HCl (0.243 g., 0.769 mmol) were reacted according to General Procedure C and triturated with methylene chloride, filtered and dried to afford the title compound (0.481 g., 92%). MS (ESI) m/z 541 [M+1]⁺.

D. 8-methoxypyridino[3,2-c]pyridine-2-carbohydrazide. Methyl 8-bromopyridino[3,4-e]pyridine-2-carboxylate (1.3 g., 4.86 mmol) was reacted with 25% sodium methoxide/methanol and allowed to heat at 80° C. for 16 hours in a screw capped flask. The solution was then decanted into methanol and HCl (g) bubbled in and the mixture heated to 75° C. for 16 hours. The solution was condensed and partitioned between 1.5M K₂CO₃ and (10%) isopropanol/chloroform (3×). The organics were dried over magnesium sulfate, filtered and solvent removed under reduced pressure. The resultant solid was purified via silica gel chromatography (10% methanol/methylene chloride) to afford the title compound (0.310 g., 29%). ¹H NMR (300 MHz, CD₃OD) δ 9.06 (s, 1H), 8.73 (d, 1H), 8.45 (s, 1H), 8.36 (d, 1H), 4.20 (s, 3H), 4.09 (s, 3H). MS (ESI) m/z 219 [M+1]⁺.

E. Methyl 8-bromopyridino[3,4-e]pyridine-2-carboxylate. 8-Bromo-1,6-naphthyridine-2-carboxylic acid (2.0 g., 7.90 mmol) was diluted with methanol and acidified with HCl(g) and heated to 70° C. Starting material consumption was monitored via LCMS. After 2 hours, the solution was condensed under reduced pressure and partitioned between 1.5 M K₂CO₃ and methylene chloride. The organics were dried over magnesium sulfate, filtered and solvent removed under reduced pressure to afford the title compound (1.55 g., 73%). MS (ESI) m/z 267[M+1]⁺, 269 [M+2]⁺.

### 5.1.31 SYNTHESIS OF [(1R)-2-AMINO-1-BENZYLETHYL](5-(6-ISOQUINOLYLY)(1,3-THIAZOL-2-YL))AMINE HYDROCHLORIDE

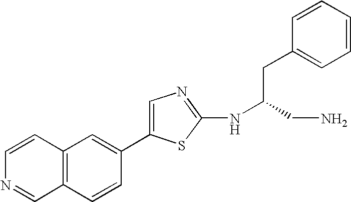

A. [(1R)-2-Amino-1-benzylethyl](5-(6-isoquinolyl)(1,3-thiazol-2-yl))amine hydro chloride. A solution of 2-[(2R)-2-({[(2-(6-isoquinolyl)-2-oxoethyl)amino]thioxomethyl}amino)-3-phenylpropyl]benzo[c]azoline-1,3-dione (crude, 7 mmol) in methane sulfonic acid (25 mL) was stirred 20 hours at 50° C. The reaction was cooled, diluted with water, neutralized with saturated aqueous sodium bicarbonate, and then diluted with dichloromethane. The resulting emulsion was filtered through celite. The organic phase separated, washed with brine, dried over anhydrous sodium sulfate, filtered, and volatiles evaporated. The material was purified using chromatography on a normal phase silica gel column with 60 to 100% ethyl acetate in hexanes. Fractions containing intermediate product were combined and the solvent evaporated. The material was dried under vacuum at 60° C., dissolved in ethanol (10 mL), and then hydrazine monohydrate (5 mL) was added. The reaction was stirred at room temperature for 1 hour and then the volatiles were evaporated. The residue was purified using reverse-phase preparatory HPLC (10-50% acetonitrile/water/0.1% TFA, over 30 min). Fractions containing clean product were combined and the volatiles were evaporated. The material was treated three times by dissolving with a solution of concentrated hydrochloric acid (2 mL) in methanol (4 mL) and then evaporating the volatiles. The resulting solids were dried under vacuum at 60° C. to give the title compound (281 mg, 9%, 2 steps). ¹H NMR (400 MHz, DMSO) δ 9.67 (s, 1H), 8.74 (d, J=8, 1H), 8.57 (d, J=6.4, 1H), 8.40 (d, J=9.2, 1H), 8.33 (d, J=6, 1H), 8.28 (d, J=8.8, 1H), 8.19 (broad s, 3H), 8.08 (s, 1H), 8.07 (s, 1H), 7.26-7.34 (m, 4H), 7.19-7.26 (m, 1H), 4.24-4.34 (m, 1H), 2.95-3.06 (m, 4H); MS (ESI) m/z 361.4 [M+1]⁺.

B. 2-[(2R)-2-({[(2-(6-Isoquinolyl)-2-oxoethyl)amino]thioxomethyl}amino)-3-phenyl propyl]benzo[c]azoline-1,3-dione. To a solution of 2-((2R)-2-amino-3-phenyl propyl)benzo[c]azoline-1,3-dione hydrochloride (2.22 g, 7.0 mmol) in dichloromethane (30 mL) was added aqueous sodium carbonate until the mixture was basic to pH paper. The organic phase was separated and the aqueous solution was extracted three times with dichloromethane. The combined organic phases were washed with brine, dried over anhydrous sodium sulfate, and filtered. The solvent was evaporated and the material was dried under vacuum at room temperature. To a solution of the residue in anhydrous THF (75 mL) was added di-2-pyridyl thionocarbonate (1.633 g, 7.0 mmol). The reaction was stirred 1 hour at room temperature. The solvent was evaporated, the material was dried under vacuum at room temperature, and dissolved in anhydrous ethanol (8 mL). To this solution was added 2-amino-1-(6-isoquinolyl)ethan-1-one hydrochloride (crude, 7.0 mmol) and a solution of anhydrous triethylamine (5.88 mL, 42.18 mmol) in anhydrous ethanol (5 mL). The mixture was heated to 60° C. with stirring for one hour in a sealed tube. The reaction mixture was cooled to room temperature, the volatiles evaporated, and the material was dried under vacuum to give the title compound (crude, 7.0 mmol). MS (ESI) m/z 509.4 [M+1]⁺.

C. 2-Amino-1-(6-isoquinolyl)ethan-1-one hydrochloride. To a solution of 33% hydrogen bromide in acetic acid (26 mL), was added acetic acid (87 mL), 1-(6-isoquinolyl)ethan-1-one (2.14 g, 12.5 mmol), and pyridinium tribromide (4.0 g, 12.5 mmol). The mixture was heated gently with warm water and stirred for 90 minutes. The reaction was diluted with ethyl ether (500 mL), the precipitate was filtered and rinsed with ethyl ether (100 mL). The material was dried under vacuum. To the resulting solids was added acetone (26 mL), water (7 mL), and sodium azide (1.45 g, 22.3 mmol). The mixture was heated to 50° C. for 30 minutes, cooled, and the volatiles evaporated. The residue was diluted with water and extracted three times with a 5% methanol in dichloromethane solution. The combined organic phases were washed with aqueous sodium bicarbonate, dried over anhydrous sodium sulfate, filtered, and the volatiles evaporated. The residue was purified using chromatography on a normal phase silica gel column with 30 to 50% ethyl acetate in hexanes. The intermediate azide (1.5 g, 7.0 mmol) was dried under vacuum and then anhydrous ethanol (35 mL) was added followed by tin(II) chloride dihydrate (4.76 g, 21.1 mmol). The mixture was stirred for 4 hours at room temperature to give the title compound as a crude solution in ethanol. (crude, 7.0 mmol). MS (ESI) m/z 187.3 [M+1]⁺.

### 5.1.32 SYNTHESIS OF [(1R)-2-AMINO-1-(BENZO[B]THIOPHEN-3-YLMETHYL)ETHYL](5-(6-ISOQUINOLYL)(1,3,4-THIADIAZOL-2-YL))AMINE

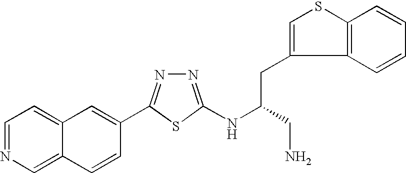

A. [(1R)-2-Amino-1-(benzo[b]thiophen-3-ylmethyl)ethyl](5-(6-isoquinolyl)(1,3,4-thiadiazol-2-yl))amine. N-[({[(1R)-1-(Benzo[b]thiophen-3-ylmethyl)-2-(1,3-dioxobenzo[c]azolidin-2-yl)ethyl]amino}thioxomethyl)amino]-6-isoquinolylcarboxamide (302 mg, 0.53 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.5% pure, (123 mg, 55%). ¹H NMR (400 MHz, DMSO-d₆) δ 9.34 (s, 1H), 8.55 (d, J=5.6 Hz, 1H), 8.26 (s, 1H), 8.15 (m, 3H), 7.97 (d, J=8.0 Hz, 1H), 7.93 (d, J=5.6 Hz, 1H), 7.51 (s, 1H), 7.45 (t, J=8.0 Hz, 1H), 7.38 (t, J=8.0 Hz, 1H), 4.07 (m, 1H), 3.19 (m, 2H), 2.77 (m, 2H). MS (ESI) m/z 418.4 [M+1]⁺.

B. N-[({[(1R)-1-(Benzo[b]thiophen-3-ylmethyl)-2-(1,3-dioxobenzo[cl azolidin-2-yl)ethyl]amino}thioxomethyl)amino]-6-isoquinolylcarboxamide. 2-((2R)-2-Amino-3-benzo[b]thiophen-3-ylpropyl)benzo[c]azolidine-1,3-dione HCl (198 mg, 0.53 mmol) was coupled with isoquinoline-6-carbohydrazide according to General Procedure C. The crude product was precipitated out of ether to give an off-white solid. MS (ESI) m/z 566.3 [M+1]⁺.

C. 2-((2R)-2-Amino-3-benzo[b]thiophen-3-ylpropyl)benzo[c]azolidine-1,3-dione HCl. N-[(1R)-1-(Benzo[b]thiophen-3-ylmethyl)-2-hydroxyethyl](tert-butoxy)carboxamide (0.67 g, 2.4 mmol) was dissolved in THF (10 mL). Then added with triphenylphosphine (740 mg, 2.8 mmol) and phthalimide (421 mg, 2.8 mmol). The reaction was cooled in an ice-bath before diisopropyl azodicarboxylate (572 mg, 2.8 mmol) was added dropwise through a syringe. The reaction was stirred at room temperature for 3 hours. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.68 g, 84% yield). MS (ESI) m/z 337.4 [M+1]⁺.

D. N-[(1R)-1-(Benzo[b]thiophen-3-ylmethyl)-2-hydroxyethyl](tert-butoxy)carboxamide. N-Boc-D-3-benzothienylalanine (1 g, 3.1 mmol) was dissolved in THF (10 mL). Lithium aluminum hydride (237 mg, 6.2 mmol) was added to the THF solution in small portions and then allowed to stirred for 1 hour. The reaction was quenched with saturated aqueous Na₂SO₄ (2 mL), and then triturated in 2-propanol. The 2-propanol mixture was filtered and then concentrated to give a colorless oil, (0.67 g, 68% yield). MS (ESI) m/z 308.4 [M+1]⁺.

### 5.1.33 SYNTHESIS OF [(1S)-2-AMINO-1-(BENZO[B]THIOPHEN-3-YLMETHYL)ETHYL](5-(6-ISOQUINOLYL)(1,3,4-THIADIAZOL-2-YL))AMINE

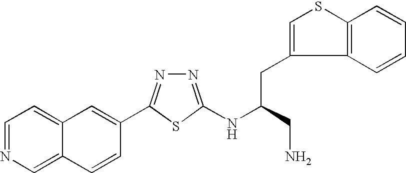

A. [(1S)-2-Amino-1-(benzo[b]thiophen-3-ylmethyl)ethyl](5-(6-isoquinolyl)(1,3,4-thiadiazol-2-yl))amine. N-[({[(1S)-1-(Benzo[b]thiophen-3-ylmethyl)-2-(1,3-dioxobenzo[c]azolidin-2-yl)ethyl]amino}thioxomethyl)amino]-6-isoquinolylcarboxamide (164 mg, 0.29 mmol) was cyclized according to General Procedure B to give an oil. The phthalimide was deprotected according to General Procedure A to give the crude amine. The amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 99.5% purity, (30 mg, 25%). ¹H NMR (400 MHz, DMSO-d₆) δ 9.34 (s, 1H), 8.55 (d, J=5.6 Hz, 1H), 8.26 (s, 1H), 8.15 (m, 3H), 7.97 (d, J=8.0 Hz, 1H), 7.93 (d, J=5.6 Hz, 1H), 7.51 (s, 1H), 7.45 (t, J=8.0 Hz, 1H), 7.38 (t, J=8.0 Hz, 1H), 4.07 (m, 1H), 3.19 (m, 2H), 2.77 (m, 2H). MS (ESI) m/z 418.1 [M+1]⁺.

B. N-[({[(1S)-1-(Benzo[b]thiophen-3-ylmethyl)-2-(1,3-dioxobenzo[c]azolidin-2-yl)ethyl]amino}thioxomethyl)amino]-6-isoquinolylcarboxamide. 2-((2S)-2-Amino-3-benzo[b]thiophen-3-ylpropyl)benzo[c]azolidine-1,3-dione HCl (100 mg, 0.53 mmol) was coupled with isoquinoline-6-carbohydrazide according to General Procedure C. The crude product was precipitated out of ether to give an off-white solid. MS (ESI) m/z 566.3 [M+1]⁺.

C. 2-((2S)-2-Amino-3-benzo[b]thiophen-3-ylpropyl)benzo[c]azolidine-1,3-dione HCl. N-[(1S)-1-(Benzo[b]thiophen-3-ylmethyl)-2-hydroxyethyl](tert-butoxy)carboxamide (0.75 g, 2.4 mmol) was dissolved in THF (10 mL), triphenylphosphine (768 g, 2.9 mmol) and phthalimide (431 mg, 2.9 mmol) was then added. The reaction was cooled in an ice-bath before diisopropyl azodicarboxylate (592 mg, 2.9 mmol) was added dropwise through a syringe. The reaction was stirred at room temperature for 3 hours. follow by removal of solvent under reduced pressure to give an oil. The oil was treated with 4N HCl in dioxane and stirred at room temperature for 1 hour. The reaction was triturated with EtOAc to give a white solid. The solid was filtered and dried under vacuum to give the product salt, (0.85 g, 93% yield). MS (ESI) m/z 337.5 [M+1]⁺.

D. N-[(1S)-1-(Benzo[b]thiophen-3-ylmethyl)-2-hydroxyethyl](tert-butoxy)carboxamide. N-Boc-L-3-benzothienylalanine (1 g, 3.1 mmol) was dissolved in THF (10 mL). Lithium aluminum hydride (237 mg, 6.2 mmol) was added to the THF solution in small portions and then allowed the reaction to stirred for 1 hour. The reaction was quenched with saturated aqueous Na₂SO₄ (2 mL), and then triturated in 2-propanol. The 2-propanol mixture was filtered and then concentrated to give a colorless oil, (0.75 g, 78% yield). MS (ESI) m/z 308.4 [M+1]⁺.

### 5.1.34 SYNTHESIS OF {(1R)-2-AMINO-1-[(2,4-DICHLOROPHENYL)METHYL]ETHYL}(5-(6-ISOQUINOLYL)(1,3,4-THIADIAZOL-2-YL))AMINE

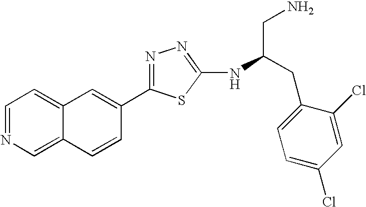

A. {(1R)-2-amino-1-[(2,4-dichlorophenyl)methyl]ethyl}(5-(6-isoquinolyl)(1,3,4-thiadiazol-2-yl))amine 2-[(2R)-2-amino-3-(2,4-dichlorophenyl)propyl]benzo[c]azolidine-1,3-dione (652 mg, 1.16 mmol) was reacted according to General Procedure A to afford a crude residue of the title compound. The sample was dissolved in 1:1 DMSO:MeOH and purified using RP-HPLC on a C18 column with 10-70% CH₃CN/H₂O/0.1% TFA to provide the title compound (340 mg, 68%). ¹H NMR (400 MHz, CD₃OD) δ 9.76 (s, 1H), 8.06-8.47 (m, 5H), 7.42 (s, 1H); 7.38 (d, J=1.2 Hz), 1H) 7.24 (m, 1H), 4.61-4.53 (m, 1H), 3.32 (d, J=7.2), 2H), 3.24-3.11 (m, 2H), 2.91 (m, 2H); ES-MS (m/z) 430[M+1]⁺.

B. 2-{(2R)-3-(2,4-dichlorophenyl)-2-[(5-(6-isoquinolyl)(1,3,4-thiadiazol-2-yl))amino]propyl}benzo[c]azolidine-1,3-dione Crude 2-[(2R)-2-amino-3-(2,4-dichlorophenyl)propyl]benzo[c]azolidine-1,3-dione (832 mg) was reacted according to General Procedure B and purified using flash chromatography, 98:2 DCM:MeOH affords a crude residue of title compound (652 mg, 81%). ES-MS (m/z) 561[M+1]⁺.

C. N-{[({(1R)-1-[(2,4-dichlorophenyl)methyl]-2-(1,3-dioxobenzo[c]azolidin-2-yl)ethyl}amino)thioxomethyl]amino}-6-isoquinolylcarboxamide. 2-[(2R)-2-amino-3-(2,4-dichloro phenyl)propyl]benzo[c]azolidine-1,3-dione (603 mg, 1.73 mmol) was reacted according to General Procedure C to afford a crude residue of the title compound (832 mg) Crude material was used in the next step without further purification. ES-MS (m/z) 579[M+1]⁺.

D. 2-[(2R)-2-amino-3-(2,4-dichlorophenyl)propyl]benzo[c]azolidine-1,3-dione. N-{(1R)-1-[(2,4-dichlorophenyl)methyl]-2-(1,3-dioxobenzo[c]azolidin-2-yl)ethyl}(tert-butoxy)carboxamide was reacted according to General Procedure D to afford the title compound as the HCl salt (603 mg, >100%). ES-MS (m/z) 350[M+1]⁺.

E. N-{(1R)-1-[(2,4-dichlorophenyl)methyl]-2-(1,3-dioxobenzo[c]azolidin-2-yl)ethyl}(tert-butoxy)carboxamide. N-{(1R)-1-[(2,4-dichlorophenyl)methyl]-2-hydroxyethyl}(tert-butoxy) carboxamide (694 mg, 2.17 mmol) was reacted according to General Procedure E. Crystals filtered from the reaction mixture afforded the title compound. (711 mg, 73%). ES-MS (m/z) 450[M+1]⁺.

F. N-{(1R)-1-[(2,4-dichlorophenyl)methyl]-2-hydroxyethyl}(tert-butoxy)carboxamide. (2R)-3-(2,4-dichlorophenyl)-2-[(tert-butoxy)carbonylamino]propanoic acid (2.0 g, 5.98 mmol) was reacted according to General Procedure E. Purification using flash chromatography, 98:2 DCM:MeOH afforded the crude title compound (850 mg, 45%). ES-MS (m/z) 321[M+1]⁺.

### 5.1.35 SYNTHESIS OF ((1R)-2-AMINO-1-{[3-(TRIFLUOROMETHYL)PHENYL]METHYL}ETHYL)(5-(6-ISOQUINOLYL)(1,3,4-THIADIAZOL-2-YL))AMINE

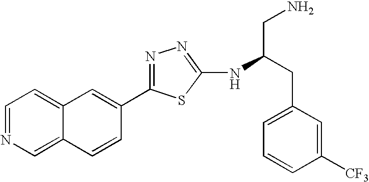

A. ((1R)-2-amino-1-{[3-(trifluoromethyl)phenyl]methyl}ethyl)(5-(6-isoquinolyl)(1,3,4-thiadiazol-2-yl))amine. 2-{(2R)-2-[(5-(6-isoquinolyl)(1,3,4-thiadiazol-2-yl))amino]-3-[3-(trifluoromethyl)phenyl]propyl}benzo[c]azolidine-1,3-dione (329 mg, 0.588 mmol) was reacted according to General Procedure A to afford a crude residue of title compound. The sample was dissolved in 1:1 DMSO:MeOH and purified using RP-HPLC on a C18 column with 10-70% CH₃CN/H₂O/0.1% TFA to provide the title compound (212 mg, 84%). ¹H NMR (400 MHz, CD₃OD) δ 9.74 (s, 1H), 8.59-8.48 (m, 5H), 7.60 (s, br, 1H); 7.57 (m, 1H), 7.48 (m, 1H), 4.49 (m, 1H), 3.24-3.11 (m, 4H); ES-MS (m/z) 430[M+1]⁺.

B. 2-{(2R)-2-[(5-(6-isoquinolyl)(1,3,4-thiadiazol-2-yl))amino]-3-[3-(trifluoromethyl)phenyl]propyl}benzo[c]azolidine-1,3-dione. Crude SN-({[((1R)-2-(1,3-dioxobenzo[c]azolidin-2-yl)-1-{[3-trifluoromethyl)phenyl]methyl}ethyl)amino]thioxomethyl}amino)-6-isoquinolylcarbox-amide (1.1 g, 1.9 mmol) was reacted according to General Procedure B and purified using flash chromatography, 98:2 DCM:MeOH to afford the title compound. (329 mg, 31%). ES-MS (m/z) 560[M+1]⁺.

C. N-({[((1R)-2-(1,3-dioxobenzo[c]azolidin-2-yl)-1-{[3-(trifluoromethyl)phenyl]methyl}ethyl)amino]thioxomethyl}amino)-6-isoquinolylcarboxamide. 2-{(2R)-2-amino-3-[3-(tri-fluoromethyl)phenyl]propyl}benzo[c]azolidine-1,3-dione (397 mg, 1.14 mmol) was reacted according to General Procedure C to afford a crude residue of the title compound (1.1 g). The crude material was used in the next step without purification. ES-MS (m/z) 578[M+1]⁺.

D. 2-{(2R)-2-amino-3-[3-(trifluoromethyl)phenyl]propyl}benzo[c]azolidine-1,3-dione. N-((1R)-2-(1,3-dioxobenzo[c]azolidin-2-yl)-1-{[3-(trifluoromethyl)phenyl]methyl}ethyl)(tert-butoxy)carboxamide (481 mg, 1.07 mmol) was reacted according to General Procedure D to afford the title compound as HCl salt (397 mg, >100%). ES-MS (m/z) 349[M+1]⁺.

E. N-((1R)-2-(1,3-dioxobenzo[c]azolidin-2-yl)-1-{[3-(trifluoromethyl)phenyl]methyl}ethyl)(tert-butoxy)carboxamide. N-((1R)-2-hydroxy-1-{[3-(trifluoromethyl)phenyl]methyl}ethyl)(tert-butoxy)carboxamide (481 mg, 1.51 mmol) was reacted according to General Procedure E and purified using flash chromatography, 98:2 DCM:MeOH to afford a crude residue of the title compound (476 mg, 70%). ES-MS (m/z) 449[M+1]⁺.

F. N-((1R)-2-hydroxy-1-{[3-(trifluoromethyl)phenyl]methyl}ethyl) (tert-butoxy)carbox amide (2R)-2-[(tert-butoxy)carbonylamino]-3-[3-(trifluoromethyl)phenyl]propanoic acid (1 g, 3.0 mmol) was reacted according to General Procedure F and purified using flash chromatography, 98:2 DCM:MeOH to afford the title compound (481 mg, 50%). ES-MS (m/z) 320[M+1]⁺.

### 5.1.36 SYNTHESIS OF {(1S)-2-AMINO-1-[(4-FLUOROPHENYL)METHYL]ETHYL}(5-(6-ISOQUINOLYL)(1,3,4-THIADIAZOL-2-YL))AMINE

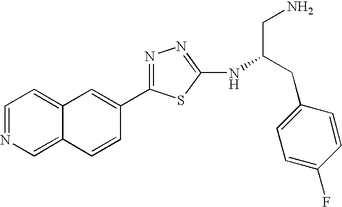

A. {(1S)-2-amino-1-[(4-fluorophenyl)methyl]ethyl}(5-(6-isoquinolyl)(1,3,4-thiadiazol-2-yl)) amine. 2-{(2S)-3-(4-fluorophenyl)-2-[(5-(6-isoquinolyl)(1,3,4-thiadiazol-2-yl))amino]propyl}benzo[c]azolidine-1,3-dione (125 mg, 0.245 mmol). Following General Procedure A afforded a crude residue of title compound. The sample was dissolved in 1:1 MSO:MeOH. Purified by RP-HPLC on a C18 column with 10-70% CH₃CN/H₂O/0.1% TFA to provide the desired product (31 mg, 33%). ¹H NMR (400 MHz, CD₃OD) δ 9.67 (s, 1H), 8.52-8.43 (m, 5H), 7.23 (m, 2H); 7.57 (m, 1H), 6.93 (m, 2H), 4.37 (m, 1H), 3.19-3.14 (m, 2H), 2.97 (d, J=7.2 Hz); ES-MS (m/z) 380[M+1]⁺.

B. 2-{(2S)-3-(4-fluorophenyl)-2-[(5-(6-isoquinolyl)(1,3,4-thiadiazol-2-yl))amino]propyl}benzo[c]azolidine-1,3-dione. Crude N-{[({(1S)-2-(1,3-dioxobenzo[c]azolidin-2-yl)-1-[(4-fluorophenyl)methyl]ethyl}amino)thioxomethyl]amino}-6-isoquinolylcarboxamide (619 mg, 1.17 mmol) was reacted according to General Procedure B and purified using flash chromatography, 98:2 DCM:MeOH to afford the title compound. (125 mg, 21%). ES-MS (m/z) 510[M+1]⁺.

C. N-{[({(1S)-2-(1,3-dioxobenzo[c]azolidin-2-yl)-1-[(4-fluorophenyl)methyl]ethyl}amino)thioxomethyl]amino}-6-isoquinolylcarboxamide. 2-[(2S)-2-amino-3-(4-fluorophenyl)propyl]benzo[c]azolidine-1,3-dione (182 mg, 0.610 mmol) was reacted according to General Procedure C to afford a crude residue of the title compound (619 mg). Crude material was used for the next step without purification. ES-MS (m/z) 528[M+1]⁺.

D. 2-[(2S)-2-amino-3-(4-fluorophenyl)propyl]benzo[c]azolidine-1,3-dione N-{(1S)-2-(1,3-dioxobenzo[c]azolidin-2-yl)-1-[(4-fluorophenyl)methyl]ethyl}(tert-butoxy)carboxamide (208 mg, 0.522 mmol) was reacted according to General Procedure D to afford the title compound as the HCl salt (182 mg). ES-MS (m/z) 299[M+1]⁺.

E. N-{(1S)-2-(1,3-dioxobenzo[c]azolidin-2-yl)-1-(4-fluorophenyl)methyl]ethyl}(tert-butoxy)carboxamide N-{(1S)-1-[(4-fluorophenyl)methyl]-2-hydroxyethyl}(tert-butoxy)carboxamide (197 mg, 0.731 mmol) was reacted according to General Procedure E and purified using flash chromatography, 98:2 DCM:MeOH to afford the title compound. (208 mg, 71%). ES-MS (m/z) 399[M+1]⁺.

F. N-{(1S)-1-[(4-fluorophenyl)methyl]-2-hydroxyethyl}(tert-butoxy)carboxamide (2S)-2-[(tert-butoxy)carbonylamino]-3-(4-fluorophenyl)propanoic acid (1.0 g, 3.53 mmol) was reacted according to General Procedure F and purified using flash chromatography, 98:2 DCM:MeOH to afford the title compound (603 mg, 63%). ES-MS (m/z) 321[M+1]⁺.

### 5.1.37 SYNTHESIS OF [(3R)-2-(5-(6-ISOQUINOLYL)(1,3,4-THIADIAZOL-2-YL))-3-1,2,3,4-TETRAHYDROISOQUINOLYL]METHYLAMINE

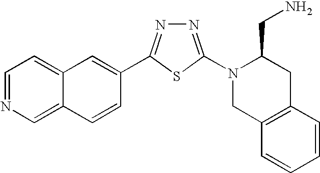

A. [(3R)-2-(5-(6-Isoquinolyl)(1,3,4-thiadiazol-2-yl))-3-1,2,3,4-tetrahydroisoquinolyl]methylamine. 2-{[(3R)-2-(5-(6-Isoquinolyl)(1,3,4-thiadiazol-2-yl))-3-1,2,3,4-tetrahydro-isoquinolyl]methyl}benzo[c]azolidine-1,3-dione (130 mg, 0.26 mmol) was deprotected according to General Procedure A to give the crude amine. The crude amine was purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to afford the title compound, 98.9% purity, (14 mg, 14%). ¹H NMR (400 MHz, CD₃OD) δ 9.23 (s, 1H), 8.46 (d, J=5.6, 1H), 8.29 (s, 1H), 8.20 (m, 2H), 7.87 (d, J=6.0 Hz, 1H), 7.23 (s, 4H), 4.65 (d, J=16.4, 1H), 4.10 (q, J=7.6 Hz, 1H), 3.31 (m, 1H), 3.20 (d, J=6.8 Hz, 1H), 3.04 (dd, J=15.6, 2.0 Hz, 1H), 2.77 (dd, J=7.2, 13.2, 2H); 2.63 (dd, J=7.2, 13.2, 2H) MS (ESI) m/z 374.1 [M+1]⁺.

B. 2-{[(3R)-2-(5-(6-Isoquinolyl)(1,3,4-thiadiazol-2-yl))-3-1,2,3,4-tetrahydro isoquinolyl]methyl}benzo[c]azolidine-1,3-dione. 2-{(2R)-2-[(5-(6-Isoquinolyl)(1,3,4-thia diazol-2-yl)) amino]-3-phenylpropyl}benzo[c]azolidine-1,3-dione (CC0390427, 210 mg, 0.4 mmol) was added with formic acid (4 mL) and 37% aq. formaldehyde (4 mL). The reaction was heated to 90° C. under N₂ for 16 hours. The resulting material was taken up in CH₂Cl₂, washed with saturated sodium bicarbonate, and brine in succession. The organic layer was dried over Na₂SO₄, filtered, and concentrated to give a slightly yellow solid. The solid was flash chromatographed with 5% MeOH-EtOAc to give the title compound, (160 mg, 74%). MS (ESI) m/z 504.4 [M+1]⁺.

### 5.1.38 SYNTHESIS OF {(1S)-2-AMINO-1-[(2,4-DICHLOROPHENYL)METHYL]ETHYL}(5-(6-ISOQUINOLYL)(1,3,4-THIADIAZOL-2-YL))AMINE

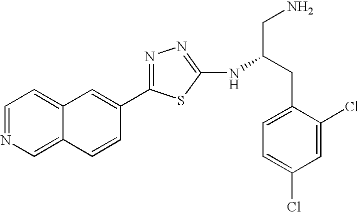

A. {(1S)-2-amino-1-[(2,4-dichlorophenyl)methyl]ethyl}(5-(6-isoquinolyl)(1,3,4-thiadiazol-2-yl))amine. 2-{(2S)-3-(2,4-dichlorophenyl)-2-[(5-(6-isoquinolyl)(1,3,4-thiadiazol-2-yl))amino]propyl}benzo[c]azolidine-1,3-dione (503 mg, 0.897 mmol) was reacted according to General Procedure A to afford a crude residue of the title compound. The sample was dissolved in 1:1 DMSO:MeOH and purified using RP-H PLC on a C18 column with 10-70% CH₃CN/H₂O/0.1% TFA to provide the title compound (322 mg, 84%). ¹H NMR (400 MHz, CD₃OD) δ 9.75 (s, 1H), 8.59-8.48 (m, 5H), 7.43 (d, J=2.4 Hz), 1H); 7.35 (d, J=8.4 Hz), 1H), 7.23 (dd, J=2.4 Hz, 7.2 Hz), 1H), 4.57 (m, 1H), 3.32-3.28 (m, 2H), 3.25-3.10 (m, 2H); ES-MS (m/z) 431[M+1]⁺.

B. 2-{(2S)-3-(2,4-dichlorophenyl)-2-[(5-(6-isoquinolyl)(1,3,4-thiadiazol-2-yl))amino]propyl}benzo[c]azolidine-1,3-dione. Crude N-{[({(1S)-1-[(2,4-dichlorophenyl)methyl]-2-(1,3-dioxobenzo[c]azolidin-2-yl)ethyl}amino) thioxomethyl]amino}-6-isoquinolyl carboxamide (606 mg, 1.05 mmol) was reacted according to General Procedure B and purified using flash chromatography, 98:2 DCM:MeOH to afford the title compound. (503 mg, 86%). ES-MS (m/z) 560[M+1]⁺.

C. N-{[({(1S)-1-[(2,4-dichlorophenyl)methyl]-2-(1,3-dioxobenzo[c]azolidin-2-yl)ethyl}amino)thioxomethyl]amino}-6-isoquinolylcarboxamide. 2-[(2S)-2-amino-3-(2,4-dichloro-phenyl)propyl]benzo[c]azolidine-1,3-dione (518 mg, 1.48 mmol) was reacted according to General Procedure C to afford a crude residue of the title compound. (606 mg). Crude material was used for the next step without purification ES-MS (m/z) 579[M+1]⁺.

D. 2-[(2S)-2-amino-3-(2,4-dichlorophenyl)propyl]benzo[c]azolidine-1,3-dione. N-{(1S)-1-[(2,4-dichlorophenyl)methyl]-2-(1,3-dioxobenzo[c]azolidin-2-yl)ethyl}(tert-butoxy)carboxamide (816 mg, 1.82 mmol) was reacted according to General Procedure D to afford the title compound as the HCl salt. ES-MS (m/z) 350[M+1]⁺.

E. N-{(1S)-1-[(2,4-dichlorophenyl)methyl]-2-(1,3-dioxobenzo[c]azolidin-2-yl)ethyl}(tert-butoxy)carboxamide. N-{(1S)-1-[(2,4-dichlorophenyl)methyl]-2-hydroxyethyl}(tert-butoxy)carboxamide (554 mg, 1.73 mmol) was reacted according to General Procedure E to afford a crude residue of the title compound. The reaction mixture was concentrated and the product was recrystallized from MeOH. ES-MS (m/z) 450[M+1]⁺.

F. N-{(1S)-1-[(2,4-dichlorophenyl)methyl]-2-hydroxyethyl}(tert-butoxy)carboxamide. (2S)-3-(2,4-dichlorophenyl)-2-[(tert-butoxy)carbonylamino]propanoic acid (1.0 g, 2.99 mmol) was reacted according to General Procedure F and purified using flash chromatography, 98:2 DCM:MeOH to afford the title compound (554 mg, 58%). ES-MS (m/z) 321[M+1]⁺.

### 5.1.39 SYNTHESIS OF ((1S)-2-AMINO-1-{[3-(TRIFLUOROMETHYL)PHENYL]METHYL}ETHYL)(5-(6-ISOQUINOLYL)(1,3,4-THIADIAZOL-2-YL))AMINE

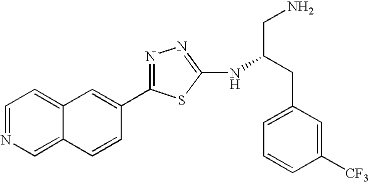

A. ((1S)-2-amino-1-{[3-(trifluoromethyl)phenyl]methyl}ethyl)(5-(6-isoquinolyl)(1,3,4-thiadiazol-2-yl))amine. 2-{(2S)-2-[(5-(6-isoquinolyl)(1,3,4-thiadiazol-2-yl))amino]-3-[3-(trifluoromethyl)phenyl]propyl}benzo[c]azoline-1,3-dione (500 mg, 0.894 mmol) following General Procedure A afforded a crude residue of product. The sample was dissolved in 1:1 DMSO:MeOH and purified using RP-HPLC on a C18 column with 10-70% CH₃CN/H₂O/0.1% TFA to provide the title compound (289 mg, 75%). ¹H NMR (400 MHz, CD₃OD) δ 9.76 (s, 1H), 8.60-8.47 (m, 5H), 7.62 (s, 1H); 7.58 (m, 1H), 7.48 (m, 2H), 4.45 (m, 1H), 3.31-3.29 (m, 2H), 3.16-3.12 (m, 2H); ES-MS (m/z) 430[M+1]⁺.

B. 2-{(2S)-2-[(5-(6-isoquinolyl)(1,3,4-thiadiazol-2-yl))amino]-3-[3-(trifluoromethyl)phenyl]propyl}benzo[c]azoline-1,3-dione. Crude N-({[((1S)-2-(1,3-dioxobenzo[c]azolidin-2-yl)-1-{[3-(trifluoromethyl)phenyl]methyl}ethyl)amino]thioxomethyl}amino)-6-isoquinolyl carboxamide (1.71 g, 2.96 mmol) was reacted according to General Procedure B and purified using flash chromatography, 98:2 DCM:MeOH to afford the title compound. ES-MS (m/z) 560[M+1]⁺.

C. N-({[((1S)-2-(1,3-dioxobenzo[c]azolidin-2-yl)-1-{[3-(trifluoromethyl)phenyl]methyl}ethyl)amino]thioxomethyl)amino)-6-isoquinolylcarboxamide. Following General Procedure D afforded a crude residue of the title compound. Crude material was used in the next step without purification. ES-MS (m/z) 579[M+1]⁺.

D. 2-{(2S)-2-amino-3-[3-(trifluoromethyl)phenyl]propyl}benzo[c]azolidine-1,3-dione. N-((1S)-2-(1,3-dioxobenzo[c]azolidin-2-yl)-1-{[3-(trifluoromethyl)phenyl]methyl}ethyl)(tert-butoxy)carboxamide (659 mg, 1.47 mmol) was reacted according to General Procedure D to afford the title compound as the HCl salt (556 mg). ES-MS (m/z) 349[M+1]⁺.

E. N-((1S)-2-(1,3-dioxobenzo[e]azolidin-2-yl)-1-{[3-(trifluoromethyl)phenyl]methyl}ethyl)(tert-butoxy)carboxamide. N-((1S)-2-hydroxy-1-{[3-(trifluoromethyl)phenyl]methyl}ethyl)(tert-butoxy)carboxamide (697 mg, 2.18 mmol) was reacted according to General Procedure E and purified using flash chromatography, 98:2 DCM:MeOH to afford the title compound (659 mg, 67%). ES-MS (m/z) 449[M+1]⁺.

F. N-((1S)-2-hydroxy-1-{[3-(trifluoromethyl)phenyl]methyl}ethyl) (tert-butoxy)carbox-amide. (2S)-2-[(tert-butoxy)carbonylamino]-3-[3-(trifluoromethyl)phenyl]propanoic acid (1.0 g, 3.0 mmol) was reacted according to General Procedure F and purified using flash chromatography, 98:2 DCM:MeOH to afford the title compound. (697 mg, 73%). ES-MS (m/z) 320[M+1]⁺.

### 5.1.40 SYNTHESIS OF [(1R)-2-AMINO-1-BENZYLETHYL](5-BENZO[d]ISOTHIAZOL-6-YL(1,3,4-THIADIAZOL-2-YL))AMINE HYDROCHLORIDE

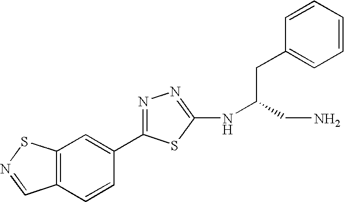

A. [(1R)-2-Amino-1-benzylethyl](5-benzo[d]isothiazol-6-yl(1,3,4-thiadiazol-2-yl))amine hydrochloride. A solution of N-[({[(1R)-2-(1,3-dioxobenzo[c]azolin-2-yl)-1-benzylethyl]amino}thioxomethyl)amino]benzo[d]isothiazol-6-ylcarboxamide (crude, 0.543 mmol) in methanesulfonic acid (3.5 mL) was heated to 50° C. for 18 hours. The reaction was cooled to room temperature, diluted with water, chilled in an ice bath, and neutralized with saturated aqueous sodium bicarbonate. The resulting solution was extracted 3 times with dichloromethane. The organic solution was dried over anhydrous sodium sulfate, filtered, and volatiles evaporated. The resulting crude intermediate was dissolved in ethanol (15 mL) and then hydrazine monohydrate (5 mL) was added. The reaction was stirred for 1 hour at room temperature. The volatiles were evaporated and the resulting material was purified using reverse-phase preparatory HPLC (10-60% acetonitrile/water/0.1% TFA, over 30 min). Fractions containing clean product were combined and the volatiles were evaporated. The material was treated 3 times by dissolving with a solution of concentrated hydrochloric acid (2 mL) in methanol (4 mL) and then evaporating the volatiles. The resulting solids were dried under vacuum at 60° C. to give the title compound (112 mg, 51%, 4 steps). ¹H NMR (400 MHz, DMSO) δ 9.17 (s, 1H), 8.64 (s, 1H), 8.40 (d, J=7.6, 11H), 8.28 (d, J=8.8, 11H), 8.13 (broad s, 3H), 7.96 (d, J=8.4, 11H), 7.27-7.36 (m, 4H), 7.20-7.27 (m, 1H), 4.23-4.33 (m, 1H), 3.20-3.50 (m, 2H), 2.98 (d, J=6.8, 2H); MS (ESI) m/z 368.3 [M+1]⁺.

B. N-[({[(1R)-2-(1,3-Dioxobenzo[c]azolin-2-yl)-1-benzylethyl]amino}thioxomethyl)amino]benzo[d]isothiazol-6-ylcarboxamide. To a solution of 2-((2R)-2-amino-3-phenylpropyl)benzo[c]azoline-1,3-dione hydrochloride (172 mg, 0.543 mmol) in dichloromethane (5 mL) was added aqueous sodium carbonate until the mixture was basic to pH paper. The organic phase was separated and the aqueous solution was extracted three times with dichloromethane. The combined organic phases were washed with brine, dried over anhydrous sodium sulfate, and filtered. The solvent was evaporated and the material was dried under vacuum at room temperature. To a solution of the residue in anhydrous THF (5.5 mL) was added a solution of di-2-pyridyl thionocarbonate (126 mg, 0.543 mmol) in anhydrous THF (8 mL). The reaction was stirred one hour at room temperature and then volatiles were evaporated. To a solution of the resulting residue in anhydrous ethanol (2.75 mL) was added benzo[d]isothiazole-6-carbohydrazide (105 mg, 0.543 mmol) and the mixture heated to 60° C. with stirring for 90 minutes. The reaction mixture was cooled to room temperature and the volatiles were evaporated. The material was dried under vacuum at 60° C. to give the title compound (crude, 0.543 mmol). MS (ESI) m/z 516.3 [M+1]⁺.

C. Benzo[d]isothiazole-6-carbohydrazide. A mixture of benzo[d]isothiazole-6-carbonitrile (116 mg, 0.724 mmol) and concentrated aqueous hydrochloric acid (4 mL) was heated to 90° C. with stirring in a sealed reaction tube for one hour. The reaction mixture was cooled, the volatiles were evaporated, and the material was dried under vacuum at 60° C. The resulting residue was dissolved in anhydrous methanol (10 mL) and then concentrated sulfuric acid (1.0 mL) was added. The reaction was stirred 90 minutes at 80° C. The reaction was cooled to room temperature, diluted with water, and neutralized with aqueous sodium carbonate. The resulting precipitate was filtered, rinsed with water, and dried under vacuum at 60° C. To a solution of the resulting solid in anhydrous methanol (3.0 mL) was added anhydrous hydrazine (0.15 mL) and the mixture heated to 65° C. with stirring for 18 hours. The reaction mixture was cooled to room temperature, the volatiles were evaporated, and the material was dried under vacuum at 60° C. to give the title compound (120 mg, 99%). MS (ESI) m/z 194.1 [M+1]⁺.

D. Benzo[d]isothiazole-6-carbonitrile. To a solution of 6-bromobenzo[d]isothiazole (205 mg, 0.96 mmol) in DMF (5 mL) was added dioxane (1 mL), tetrakis(triphenylphosphine)palladium(0) (167 mg, 0.144 mmol), and zinc cyanide (113 mg, 0.958 mmol). The reaction was heated to 95° C. with stirring for 2 hours, cooled, and the dioxane evaporated. The resulting solution was diluted with water and brine then extracted four times with dichloromethane. The organic solution was dried over anhydrous sodium sulfate, filtered, and volatiles evaporated. The resulting material was purified using chromatography on a normal phase silica gel column with 0 to 10% ethyl acetate in hexanes. Fractions containing clean product were combined and the solvent evaporated. The material was dried under vacuum at room temperature to give the title compound (118 mg, 77%).

E. 6-Bromobenzo[d]isothiazole. To a solution of 4-bromo-2-fluorobenzaldehyde (2.0 g, 9.85 mmol) in 2-methoxyethanol (10 mL) was added sulfur (316 mg, 9.85 mmol), and saturated aqueous ammonium hydroxide (10 mL). The materials were heated to 160° C. with stirring in a sealed reaction tube for 18 hours. The crude reaction was diluted with water and extracted three times with dichloromethane. The combined organic phases were dried over anhydrous sodium sulfate, filtered, and the volatiles evaporated. The residue was purified using chromatography on a normal phase silica gel column with 0 to 5% ethyl acetate in hexanes. Fractions containing clean product were combined and the solvent evaporated. The material was dried under vacuum at room temperature to give the title compound (211 mg, 10%). MS (ESI) m/z (214 and 216) [M+1]⁺.

### 5.1.41 SYNTHESIS OF [(1R)-2-AMINO-1-BENZYLETHYL][5-(4-ETHYL(6-ISOQUINOLYL))(1,3,4-THIADIAZOL-2-YL)]AMINE HYDROCHLORIDE

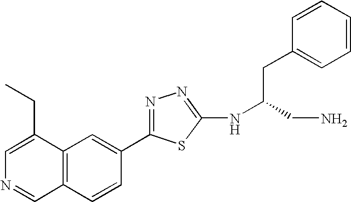

A. [(1R)-2-Amino-1-benzylethyl][5-(4-ethyl(6-isoquinolyl))(1,3,4-thiadiazol-2-yl)]amine hydrochloride. A solution of N-[({[(1R)-2-(1,3-dioxobenzo[c]azolin-2-yl)-1-benzylethyl]amino}thioxomethyl)amino](4-ethyl(6-isoquinolyl))carboxamide (crude, 0.465 mmol) in methanesulfonic acid (2 mL) was heated to 40° C. for 4 hours. The reaction was cooled to room temperature, diluted with water, chilled in an ice bath, and neutralized with saturated aqueous sodium bicarbonate. The resulting solution was extracted three times with dichloromethane. The organic solution was dried over anhydrous sodium sulfate, filtered, and volatiles evaporated. The resulting crude intermediate was dissolved in anhydrous ethanol (5 mL) and then anhydrous hydrazine (0.125 mL, 4.65 mmol) was added. The reaction was stirred for 3 hours at room temperature. The volatiles were evaporated and the resulting material was purified using reverse-phase preparatory HPLC (10-60% acetonitrile/water/0.1% TFA, over 30 min). Fractions containing 90% pure product were combined and the volatiles were evaporated. The resulting material was purified using reverse-phase semi-preparatory HPLC (10-50% acetonitrile/water/0.1% TFA, over 30 min). Fractions containing pure product were combined and the volatiles were evaporated. The material was treated 3 times by dissolving with a solution of concentrated hydrochloric acid (2 mL) in methanol (2 mL) and then evaporating the volatiles. The resulting solids were dried under vacuum at 60° C. to give the title compound (13 mg, 7%, 4 steps). ¹H NMR (400 MHz, DMSO) δ 9.60 (s, 1H), 8.61 (d, J=8.4, 1H), 8.54 (s, 1H), 8.48 (d, J=8.8, 1H), 8.42 (s, 1H), 8.33 (d, J=8.4, 1H), 8.11 (broad s, 3H), 7.26-7.35 (m, 4H), 7.20-7.26 (m, 1H), 4.25-4.35 (m, 1H), 3.22 (q, J=7.6, 2H), 3.09 (t, J=6, 2H), 2.99 (d, J=7.2, 2H), 1.36 (t, J=7.2, 3H); MS (ESI) m/z 390.4 [M+1]⁺.

B. N-[({[(1R)-2-(1,3-Dioxobenzo[c]azolin-2-yl)-1-benzylethyl]amino}thioxomethyl)amino](4-ethyl(6-isoquinolyl))carboxamide. The title compound was prepared as described in Example 5.1.40, Step B using 2-((2R)-2-amino-3-phenylpropyl)benzo[c]azoline-1,3-dione hydrochloride (147 mg, 0.465 mmol) and 4-ethylisoquinoline-6-carbohydrazide (100 mg, 0.465 mmol) to give the title compound (crude, 0.465 mmol). MS (ESI) m/z 538 [M+1]⁺.

C. 4-Ethylisoquinoline-6-carbohydrazide. To a solution methyl 4-vinylisoquinoline-6-carboxylate (130 mg, 0.610 mmol) in anhydrous ethanol (3.0 mL) was added anhydrous hydrazine (0.40 mL) and the mixture heated to 80° C. with stirring for 18 hours. The reaction mixture was cooled to room temperature, the volatiles were evaporated, and the material was dried under vacuum at 60° C. to give the title compound (130 mg, 99%). MS (ESI) m/z 216 [M+1]⁺.

D. Methyl 4-vinylisoquinoline-6-carboxylate. To a solution of methyl 4-[(trifluoromethyl)sulfonyloxy]isoquinoline-6-carboxylate (270 mg, 0.81 mmol) in 1-methyl-2-pyrrolidinone (3.2 mL) was added anhydrous lithium chloride (103 mg, 2.43 mmol), tris(dibenzylideneacetone)dipalladium(0) (74.5 mg, 0.08 mmol), and tri-2-furylphosphine (28.5 mg, 0.123 mmol). The mixture was stirred for 30 seconds then tributyl(vinyl)tin (0.475 mL, 1.62 mmol) was added. The mixture was purged with nitrogen and heated to 60° C. in a sealed reaction tube. After stirring for 90 minutes the reaction was cooled. The crude material was purified by chromatography on a normal phase silica gel column with 20-30% ethyl acetate in hexanes. Fractions containing product were combined and the solvent evaporated. Repeated chromatography could not remove all of the tributyltin byproduct. The material was dried under vacuum at room temperature to give the title compound (133 mg, 80%). MS (ESI) m/z 214.3 [M+1]⁺.

### 5.1.42 SYNTHESIS OF [(1R)-2-AMINO-1-BENZYLETHYL][5-(4-METHOXY(6-ISOQUINOLYL))(1,3,4-THIADIAZOL-2-YL)]AMINE HYDROCHLORIDE

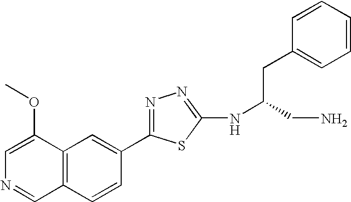

A. [(1R)-2-Amino-1-benzylethyl][5-(4-methoxy(6-isoquinolyl))(1,3,4-thiadiazol-2-yl)]amine hydrochloride. A solution of N-[({[(1R)-2-(1,3-dioxobenzo[c]azolin-2-yl)-1-benzylethyl]amino}thioxomethyl)amino](4-methoxy(6-isoquinolyl))carboxamide (crude, 0.083 mmol) in methanesulfonic acid (0.5 mL) was heated to 60° C. for 90 minutes. The reaction was cooled to room temperature, diluted with water, chilled in an ice bath, and neutralized with saturated aqueous sodium bicarbonate. The resulting solution was extracted 4 times with a solution of 5% methanol in dichloromethane. The organic solution was dried over anhydrous sodium sulfate, filtered, and volatiles evaporated. The resulting crude intermediate was dissolved in ethanol (1.0 mL) and then hydrazine monohydrate (0.20 mL) was added. The reaction was stirred for 1 hour at room temperature. The volatiles were evaporated and the resulting material was purified using reverse-phase semi-preparatory HPLC (0-40% acetonitrile/water/0.1% TFA, over 30 min). Fractions containing pure product were combined and the volatiles were evaporated. The material was treated three times by dissolving with a solution of concentrated hydrochloric acid (1 mL) in methanol (1 mL) and then evaporating the volatiles. The resulting solids were dried under vacuum at 60° C. to give the title compound (21 mg, 58%, 4 steps). ¹H NMR (400 MHz, DMSO) δ 9.28 (s, 1H), 8.53 (d, J=8.4, 1H), 8.28-8.42 (m, 4H), 8.07 (broad s, 3H), 7.28-7.35 (m, 4H), 7.19-7.25 (m, 1H), 4.24-4.34 (m, 1H), 4.16 (s, 3H), 3.02-3.10 (m, 2H), 2.98 (d, J=6.8, 2H); MS (ESI) m/z 392.4 [M+1]⁺.

B. N-[({[(1R)-2-(1,3-Dioxobenzo[c]azolin-2-yl)-1-benzylethyl]amino}thioxomethyl)amino](4-methoxy(6-isoquinolyl))carboxamide. The title compound was prepared as described in Example 5.1.40, Step B using 2-((2R)-2-amino-3-phenylpropyl)benzo[c]azoline-1,3-dione hydrochloride (26 mg, 0.083 mmol) and 4-methoxyisoquinoline-6-carbohydrazide (18 mg, 0.083 mmol) to give the title compound (crude, 0.083 mmol). MS (ESI) m/z 540.4 [M+1]⁺.

C. 4-Methoxyisoquinoline-6-carbohydrazide. To a solution of methyl 4-methoxyisoquinoline-6-carboxylate (33 mg, 0.151 mmol) in anhydrous ethanol (1.5 mL) was added anhydrous hydrazine (0.15 mL) and the mixture heated to 80° C. with stirring for 2 hours. The reaction mixture was cooled in a ice water bath, the resulting precipitate filtered, rinsed with cold ethanol, and dried under vacuum at 60° C. to give the title compound (19 mg, 58%). MS (ESI) m/z 218.4 [M+1]⁺.

D. Methyl 4-methoxyisoquinoline-6-carboxylate. To a solution of 6-bromo-4-methoxyisoquinoline (103 mg, 0.433 mmol) in DMF (1.8 mL) was added dioxane (0.36 mL), tetrakis(triphenylphosphine)palladium(0) (75 mg, 0.065 mmol), and zinc cyanide (51 mg, 0.433 mmol). The reaction was heated to 95° C. with stirring for 90 minutes and cooled. The resulting solution was diluted with dichloromethane and filtered. The volatiles were evaporated from the filtrate. The resulting residue was purified using chromatography on a normal phase silica gel column with 0 to 50% ethyl acetate in hexanes followed by 0 to 5% methanol in dichloromethane. Fractions containing product with triphenylphosphine oxide byproduct were combined and the solvent evaporated. The material was dried under vacuum at room temperature to give 117 mg of a solid. To a solution of the intermediate 4-methoxyisoquinoline-6-carbonitrile in anhydrous methanol (1.0 mL) was added anhydrous acetyl chloride (4.0 mL, excess). The reaction was stirred at room temperature for 4 hours. The volatiles were evaporated, and the material was dried under vacuum. The resulting residue was dissolved in anhydrous methanol (1.0 mL) and then concentrated sulfuric acid (0.04 mL) was added. The reaction was stirred 18 hours at 80° C. The reaction was cooled to room temperature, diluted with water, and neutralized with aqueous sodium carbonate. The solution was extracted three times with dichloromethane. The combined organic phases were washed with brine and the volatiles were evaporated. The residue was purified using reverse-phase preparatory HPLC (10-60% acetonitrile/water/0.1% TFA, over 30 min). Fractions containing product were combined and the solution made basic with aqueous ammonium hydroxide. The acetonitrile was evaporated and the solution was extracted 3 times with dichloromethane. The combined organic phases were dried over anhydrous sodium sulfate, filtered, and volatiles evaporated. The material was dried under vacuum to give the title compound (34 mg, 36%, 3 steps). MS (ESI) m/z 218 [M+1]⁺.

E. 6-Bromo-4-methoxyisoquinoline. To a solution of 6-bromoisoquinolin-4-ol (293 mg, 1.31 mmol) in DMF (6 mL) was added methanol (0.6 mL), and (trimethylsilyl)diazomethane (0.658 mL, 1.31 mmol, 2.0 M in hexanes). The reaction was stirred at room temperature for 90 minutes then additional (trimethylsilyl)diazomethane (1.32 mL, 2.62 mmol, 2.0 M in hexanes) was added. The reaction was stirred at room temperature for 60 minutes, quenched with acetic acid (3 mL), and stirred an additional 20 minutes. The mixture was neutralized with saturated aqueous sodium bicarbonate and extracted three times with dichloromethane. The combined organic layers were washed with brine and dried over anhydrous sodium sulfate. The solution was filtered and the volatiles were evaporated. The resulting material was purified using chromatography on a normal phase silica gel column with 0 to 45% ethyl acetate in hexanes. Fractions containing clean product were combined and the solvent evaporated. The material was dried under vacuum at room temperature to provide the title compound (312 mg, 34%). MS (ESI) m/z 238 [M+1]⁺ and MS (ESI) m/z 238 [M+1]⁺.

### 5.1.43 SYNTHESIS OF (2S)-1-(4-(6-ISOQUINOLYL)(1,2,3-TRIAZOLYL))-3-PHENYLPROP-2-YLAMINE

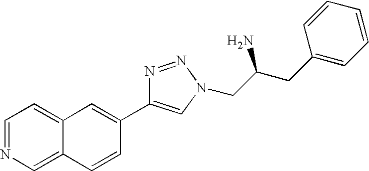

A. (2S)-1-(4-(6-Isoquinolyl)(1,2,3-triazolyl))-3-phenylprop-2-ylamine. A solution of N-[(1S)-2-(4-(6-isoquinolyl)(1,2,3-triazolyl))-1-benzylethyl](tert-butoxy)carboxamide (0.1 g, 0.23 mmol) and trifluoroacetic acid (1 mL) were reacted in dichloromethane (2 mL) for 2 hours at room temperature. The volatiles were removed under reduced pressure and the crude product was purified using reverse-phase semi-preparatory HPLC (10-50% acetonitrile+0.1% TFA in H₂O+0.1% TFA, over 30 min). Fractions containing clean product were pooled and volatiles were removed under reduced pressure, to afford the desired product, 97.6% purity (48 mg, 63%). ¹H NMR (400 MHz, DMSO) δ 13.41 (br, 1H), 9.47 (s, 1H), 8.67 (s, 1H), 8.63 (d, J=8.0 Hz, 1H), 8.48 (m, 2H), 8.34 (s, 1H), 8.07 (m, 1H), 7.36 (m, 2H), 7.29 (m, 2H), 4.37 (m, 1H), 3.26 (dd, J=5.8 and 13.8 Hz, 1H), 3.13 (dd, J=8.0 and 13.8 Hz), 1H); ES-MS (m/z) 330.3[M+1]⁺.

B. N-[(1S)-2-(4-(6-Isoquinolyl)(1,2,3-triazolyl))-1-benzylethyl](tert-butoxy)carboxamide. A solution of N-[(1S)-2-azido-1-benzylethyl](tert-butoxy) carboxamide (0.1 g, 0.36 mmol), 6-ethynylisoquinoline (55 mg, 0.36 mmol), sodium ascorbate (7.1 mg, 0.03 mmol) and copper sulfate-penta hydrate (1 mg, 0.003 mmol) in 1:1 t-butanol:water (2 mL) were reacted according to Example 5.1.51, Step B. The reaction mixture was poured into ice. The precipitate was collected, rinsed with water and dichloromethane, and dried under vacuum to provide the desired product (100 mg, 64%) as a white solid. ES-MS (m/z) 430.2[M+1]⁺.

C. N-[(1S)-2-Azido-1-benzylethyl](tert-butoxy)carboxamide. A solution of N-[(1S)-2-hydroxy-1-benzylethyl](tert-butoxy)carboxamide (2 g, 7.95 mmol) in dichloromethane (200 mL) at 0° C. was treated with triethylamine (1.80 mL, 12.72 mmol) and mesyl chloride (0.925 mL, 11.93 mmol), stirred for 1 hour and washed with water and brine, dried (MgSO₄), filtered and concentrated. The crude product was used without further purification in the next step. To the oil obtained as previously described (1.5 g, 4.57 mmol) in anhydrous dimethylformamide (50 mL) was added sodium azide (1.48 g, 22.86 mmol). The reaction mixture was heated at 60° C. for 18 hours and cooled to room temperature. The solvent was removed under reduced pressure, and the residue was dissolved in ethyl acetate, washed with water and brine, dried (MgSO₄), filtered and concentrated. The residual oil was purified using flash column chromatography on silica gel with 30% ethyl acetate/hexanes to provide the desired product (1.15 g, 91%) as a white solid. ES-MS (m/z) 277.1 [M+1]⁺.

### 5.1.44 SYNTHESIS OF (2S)-4-(4-(6-ISOQUNOLYL)(1,2,3-TRIAZOLYL))-1-PHENYLBUT-2-YLAMINE

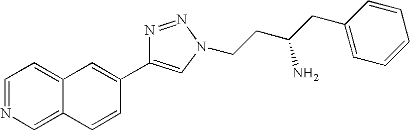

A. (2S)-4-(4-(6-Isoquinolyl)(1,2,3-triazolyl))-1-phenylbut-2-ylamine.

A solution of N-[(1S)-3-(4-(6-isoquinolyl)(1,2,3-triazolyl))-1-benzylpropyl](tert-butoxy)carboxamide (165 mg, 0.37 mmol) and trifluoroacetic acid (1 mL) were reacted in dichloromethane (2 mL) for 3.5 hours at room temperature. The volatiles were removed under reduced pressure and the crude product was purified using reverse-phase semi-preparatory HPLC (10-50% acetonitrile+0.1% TFA in H₂O+0.1% TFA, over 30 min). Fractions containing clean product were neutralized with aqueous sodium bicarbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 96.1% purity (12 mg, 10%). ¹H NMR (400 MHz, DMSO) δ. ES-MS (m/z) 344.4[M+1]⁺.

B. N-[(1S)-3-(4-(6-Isoquinolyl)(1,2,3-triazolyl))-1-benzylpropyl](tert-butoxy)carboxamide. A solution of N-[(1S)-3-azido-1-benzylpropyl](tert-butoxy) carboxamide (0.19 g, 0.65 mmol), 6-ethynylisoquinoline (0.1 g, 0.65 mmol), sodium ascorbate (13 mg, 0.06 mmol) and copper sulfate-penta hydrate (2 mg, 0.006 mmol) in 1:1 t-butanol:water (6 mL) were reacted according to Example 5.1.51, Step B. The reaction mixture was poured into ice. The precipitate was collected, rinsed with water and dichloromethane, and dried under vacuum to provide the desired product (220 mg, 76%) as a white solid. ES-MS (m/z) 444.2[M+1]⁺.

C. N-[(1S)-3-Azido-1-benzylpropyl](tert-butoxy)carboxamide. A solution of N-[(1S)-3-hydroxy-1-benzylpropyl](tert-butoxy)carboxamide (0.57 g, 2.16 mmol), methanesulfonyl chloride (0.18 mL, 2.38 mmol) and triethylamine (0.33 mL, 2.38 mmol) in dichloromethane (10 mL) were reacted according to Example 5.1.54, Step C. The resulting oil was treated with sodium azide (0.7 g, 10.8 mmol) in DMF (10 mL) according to Example 5.1.54, Step C, to yield the title compound (370 mg, 59%). ES-MS (m/z) 290.5[M+1]⁺.

D. N-[(1S)-3-Hydroxy-1-benzylpropyl](tert-butoxy)carboxamide. A solution of methyl (3S)-3-[(tert-butoxy)carbonylamino]-4-phenylbutanoate (0.66 mg, 2.26 mmol) in anhydrous THF (8 mL) at 0° C. was treated with LiBH₄ (150 mg, 6.78 mmol), stirred 1 hour at 0° C. and 2 hours at room temperature, quenched with saturated aqueous NH₄Cl, and extracted twice with ethyl acetate. The combined organic fractions were washed with water and brine, dried (MgSO₄), filtered and concentrated. The crude alcohol (575 mg) was used without purification in the next step. ES-MS (m/z) 266.1 [M+1]⁺.

E. Methyl (3S)-3-[(tert-butoxy)carbonylamino]-4-phenylbutanoate. A mixture of (3S)-3-[(tert-butoxy)carbonylamino]-4-phenylbutanoic acid (1 g, 3.57 mmol), K₂CO₃ (0.54 g, 3.93 mmol) and methyl iodide (290 mL, 4.64 mmol) in acetone (30 mL) was allowed to stir at room temperature for 1 hour, diluted with water and extracted twice with ethyl acetate. The combined organic fractions were washed with water and brine, dried (MgSO4), filtered and concentrated. The residual oil was purified using flash column chromatography on silica gel with 40% ethyl acetate/hexanes to provide the desired product (0.660 g, 63%). ES-MS (m/z) 294.1[M+1]⁺.

### 5.1.45 SYNTHESIS OF 4-(5-(6-ISOQUINOLYL)(1,3,4-THIADIAZOL-2-YL))-1-PHENYLBUT-2-YLAMINE

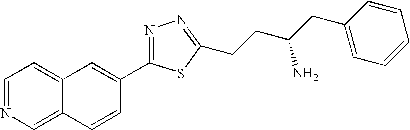

A. 4-(5-(6-isoquinolyl)(1,3,4-thiadiazol-2-yl))-1-phenylbut-2-ylamine To a solution of (4R)-4-amino-N-(6-isoquinolylcarbonylamino)-5-phenylpentanamide (0.240 g, 0.52 mmol) in pyridine (5 mL) was added 1.1 eq of phosphorus pentasulfide (0.276 g, 0.62 mmol) and the reaction was stirred at 100° C. for 2.5 hours. The reaction mixture was poured into 10 mL of water and then extracted into EtOAc (2×10 mL), dried over sodium sulfate and concentrated. The crude mixture was then dissolved in 3 mL of DCM and treated with 2 mL of TFA at 0° C. The reaction was then stirred at room temperature for 30 minutes to remove the Boc group. After concentrating the reaction mixture it was purified using preparatory HPLC to give the title compound 21 mg, (11%). ¹H NMR (DMSO, d₆) δ 9.59 (s, 1H), 8.69-8.64 (m, 2H), 8.43-8.33 (m, 2H), 8.21-8.19 (d, 1H), 7.97 (m, 3H), 7.38-7.27 (m, 4H), 3.62-3.54 (m, 1H), 3.40-3.28 (m, 2H), 3.01-2.89 (m, 2H), 2.08-2.03 (m, 2H); ES-MS (m/z) 361[M+1]⁺.

### 5.1.46 SYNTHESIS OF 4-(4-(6-ISOQUNOLYL)(1,2,3-TRIAZOLYL))-3-BENZYLBUTYLAMINE

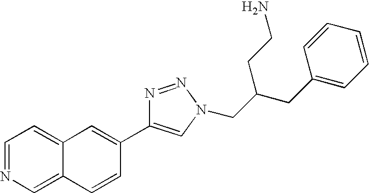

A. 4-(4-(6-Isoquinolyl)(1,2,3-triazolyl))-3-benzylbutylamine. A solution of (tert-butoxy)-N-[4-(4-(6-isoquinolyl)(1,2,3-triazolyl))-3-benzylbutyl]carboxamide (0.25 g, 0.54 mmol) and trifluoroacetic acid (2 mL) in dichloromethane (5 mL) were reacted for 1.5 hours. The volatiles were removed under reduced pressure and the crude product was purified using reverse-phase semi-preparatory HPLC (15-80% acetonitrile+0.1% TFA in H₂O+0.1% TFA, over 30 minutes). Fractions containing clean product were neutralized with aqueous sodium bicarbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.9% purity (95 mg, 48%). ¹H NMR (400 MHz, CD₃OD) δ 9.26 (s, 1H); 8.56 (s, 1H); 8.48 (d, J=8.0 Hz, 1H); 8.42 (s, 1H); 8.22-8.20 (m, 2H); 7.91 (d, J=8.0 Hz, 1H); 7.32-7.20 (m, 5H); 4.49-4.47 (m, 1H); 2.78-2.71 (m, 4H); 2.57-2.54 (m, 2H); 1.59-1.54 (m, 2H). ES-MS (m/z) 358.3[M+1]⁺.

B. (tert-Butoxy)-N-[4-(4-(6-isoquinolyl)(1,2,3-triazolyl))-3-benzylbutyl]carboxamide. A solution of N-[4-azido-3-benzylbutyl](tert-butoxy) carboxamide (0.4 g, 1.3 mmol), 6-ethynylisoquinoline (0.2 g, 1.3 mmol), sodium ascorbate (26 mg, 0.13 mmol) and copper sulfate-penta hydrate (3.3 mg, 0.013 mmol) in 1:1 t-butanol:water (12 mL) were reacted according to Example 5.1.51, Step B. The reaction mixture was poured into ice. The reaction mixture was extracted with EtOAc, and the pooled organic fractions were successively washed with water and brine, dried over magnesium sulfate, filtered and solvent removed under reduced pressure. Purification using silica gel flash column chromatography (100% EtOAc) provided the title compound, (0.25 g, 42%) as a white solid. ES-MS (m/z) 458.0[M+1]⁺.

C. N-[4-Azido-3-benzylbutyl](tert-butoxy)carboxamide. A solution of (tert-butoxy)-N-[4-hydroxy-3-benzylbutyl]carboxamide (0.48 g, 1.7 mmol), methanesulfonyl chloride (0.14 mL, 1.88 mmol) and triethylamine (0.26 mL, 1.88 mmol) in dichloromethane were reacted according to Example 5.1.54, Step C. The resulting oil was treated with sodium azide (0.55 g, 8.55 mmol) in DMF (5 mL) according to Example 5.1.54, Step C, to yield the title compound (400 mg, 77%). ES-MS (m/z) 305.2[M+1]⁺.

D. (tert-Butoxy)-N-[4-hydroxy-3-benzylbutyl]carboxamide. Sodium borohydride (405 mg, 10.7 mmole) was added to a solution tert-butyl 2-oxo-3-benzylpyrrolidinecarboxylate (405 mg, 2.7 mmole) in anhydrous THF (5 mL). After 12 hours, more sodium borohydride was added (405 mg, 2.7 mmole). After 3 additional hours, the reaction was carefully quenched with an aqueous solution of ammonium chloride, extracted with EtOAc, dried (MgSO₄), filtered, and the volatiles were removed under reduced pressure. The crude product was purified using flash chromatography on silica gel (10-50% EtOAc in hexanes) to provide the desired product (510 mg, 68%). MS (ESI) m/z 179.1 [M-Boc].

E. tert-Butyl 2-oxo-3-benzylpyrrolidinecarboxylate. At −78° C., to a solution of LiHMDS (1.0 M in THF, 22.6 mL, 22.6 mmol) in THF (60 mL) was added a solution of tert-butyl 2-oxopyrrolidinecarboxylate (4 g, 21.6 mmol) in THF (30 mL). After 25 minute, benzyl bromide (2.56 mL, 21.6 mmol) was added. Stirring was continued for 2 hours and the temperature was then raised to 0° C. over 2 hours. The reaction mixture was quenched with aqueous NH4Cl and extracted with EtOAc, dried (MgSO₄), filtered, and the volatiles were removed under reduced pressure. The crude product was purified using flash chromatography on silica gel (0-15% EtOAc in hexanes) to provide the desired product (1.23 g, 21%). MS (ESI) m/z 276.2 [M+1]⁺.

### 5.1.47 SYNTHESIS OF 2-(5-(6-ISOQUINOLYL)(1,3,4-THIADIAZOL-2-YL))-3-PHENYLPROPYLAMINE HYDROCHLORIDE

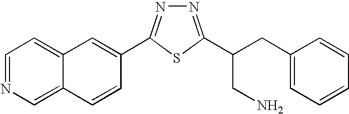

A. 2-(5-(6-Isoquinolyl)(1,3,4-thiadiazol-2-yl))-3-phenylpropylamine HCl. 3-[(tert-Butoxy)carbonylamino]-N-(6-isoquinolylcarbonylamino)-2-benzylpropanamide (672 mg, 1.5 mmol was dissolved in THF (20 mL), Lawesson's Reagent (1.2 g, 3 mmol) was added and the reaction was heated under N₂ for 16 hours. The crude reaction was extracted with EtOAc and water, follow with brine. The organic layer was dried over Na₂SO₄, filtered, and concentrated to give an oil. The oil was loaded onto preparatory thin-layer chromatography plates (2 mm) and developed with 80% EtOAc-hexane (0.5 g/plate). A blue band under the ultraviolet lamp was isolated and washed off the silica gel with EtOAc. The crude product was concentrated and then further purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to a white solid. The solid was dissolved in MeOH (5 mL), added with 4N HCl-dioxane, and then stirred for 2 hours to remove the Boc group. The product was triturated with ether and then filtered to give a white solid, 99.2% purity, (107 mg, 16% yield). ¹H NMR (400 MHz, CD₃OD) δ 9.82 (s, 1H), 8.83 (s, 1H), 8.60 (m, 4H), 7.25 (m, 5H), 4.10 (m, 1H), 3.61 (dd, J=12.8, 9.2 Hz, 1H), 3.46 (dd, J=13.2, 4.8 Hz, 1H), 3.30 (dd, J=14.0, 5.6 Hz, 1H), 3.16 (dd, J=13.6, 8.8 Hz, 1H). MS (ESI) m/z 347.1 [M+1]⁺. Anal. Calcd. for C₂₀H₁₈N₄S-2HCl, C, 56.31; H, 4.91; N, 13.13. Found, C, 56.29; H, 4.58; N, 12.92.

B. 3-[(tert-Butoxy)carbonylamino]-N-(6-isoquinolylcarbonylamino)-2-benzylpropanamide. Isoquinoline-6-carbohydrazide (281 mg, 1.5 mmol), 3-[(tert-butoxy)carbonylamino]-2-benzylpropanoic acid (419 mg, 1.5 mmol), O-(benzotriazol-1-yl)-N,N,N′,N′-tetramethyluronium hexafluorophosphate (HBTU, 569 mg, 1.5 mmol), diisopropylethylamine (581 mg, 4.5 mmol), and DMF (5 mL) were added together and stirred for 3 hours. The crude reaction was added with dichloromethane and extracted with sat. NaHCO₃, water, and brine in succession. The organic layer was dried over Na₂SO₄, filtered, and solvent removed under reduced pressure to give an oil. The oil was flash chromatographed with 5% MeOH-EtOAc. The product fractions were combined and concentrated to give an oil, (320 mg, 48%). MS (ESI) m/z 449.3 [M+1]⁺.

C. 3-[(tert-Butoxy)carbonylamino]-2-benzylpropanoic acid. Ethyl trans-α-cyanocinnamate (2 g, 10 mmol), di-tert-butyl dicarbonate (2.6 g, 12 mmol), Raney Nickel (1 mL slurry), and MeOH (20 mL) were added together and shaken in a Parr hydrogenator under 40 psi of H₂ for 20 hours. The catalyst was filter off through celite and rinsed with MeOH. The filtrate was added with NaOH (4 g, 100 mmol) and then stirred for 2 hours at room temperature to give the acid. The solvent was removed in vacuo to give an oil. The oil was diluted with water (100 mL) and then neutralized with 1N HCl. The aq. solution was extracted with EtOAc. The organic layer was subsequently washed with brine, dried over Na₂SO₄, filtered, and solvent removed under reduced pressure to give the acid as an oil, (2 g, 66%). MS (ESI) m/z 280.3 [M+1]⁺.

### 5.1.48 SYNTHESIS OF 2-[3-(6-ISOQUINOLYL)-5-(4-PHENYLBUTYL)-1,2,4-TRIAZOL-4-YL]ETHYLAMINE

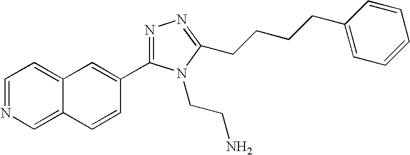

A. 2-[3-(6-isoquinolyl)-5-(4-phenylbutyl)-1,2,4-triazol-4-yl]ethylamine. (Tert-butoxy)-N-{2-[3-(6-isoquinolyl)-5-(4-phenylbutyl)(1,2,4-triazol-4-yl)]ethyl}carboxamide (0.102 g., 0.216 mmol) and 4N HCl/Dioxane were reacted according to General Procedure D to afford the title compound as the di-HCl salt (0.72 g., 76%). ¹H NMR (400 MHz, CDCl₃) δ 9.23 (s, 1H), 8.49 (d, 1H), 8.09 (d, 2H), 7.76 (dd, 2H), 7.21 (t, 2H), 0.12 9m, 3H), 4.10 (t, 2H), 3.30 (s, 2H), 2.81 (t, J=7.19, 2H), 2.75 (t, J=7.19, 2H), 2.65 (t, J=7.19., 2H), 1.86 (m, J=6.79, 2H), 1.75 (m, J=6.79, 2H). MS (ESI) m/z 372[M+1]⁺.

B. (Tert-butoxy)-N-{2-[3-(6-isoquinolyl)-5-(4-phenylbutyl)(1,2,4-triazol-4-yl)]ethyl}carboxamide. To a solution of isoquinoline-6-carbohydrazide (0.175 g., 0.935 mmol) and (tert-butoxy)-N-{2-[(5-phenyl-1-thioxopentyl)amino]ethyl}carboxamide (0.345 g., 1.03 mmol) in methanol was added potassium carbonate (0.258 g., 1.87 mmol). The solution was allowed to heat at 85° C. for 16 hours. The reaction was judged to be complete by thin layer chromatography (10% MeOH/CH₂Cl₂). The solution was condensed under reduced pressure and partitioned between water and ethyl acetate (3×). The organics were dried over magnesium sulfate, filtered and solvent removed under reduced pressure. The resultant oil was purified via preparative HPLC (15-80% acetonitrile/water, 20 mL/min.) to afford the title compound (0.130 g., 29%). MS (ESI) m/z 472 [M+1]⁺.

C. (tert-butoxy)-N-{2-[(5-phenyl-1-thioxopentyl)amino]ethyl}carboxamide. To a solution of N-{2-[(tert-butoxy)carbonylamino]ethyl}-5-phenylpentanamide (3.29 g., 10.28 mmol) in toluene (80 mL) was added Lawesson's reagent (4.57 g., 11.308 mmol). The solution was heated to 60° C. for 16 hours. The solution was filtered through a frit and the filtrant condensed under reduced pressure to give an oil. The oil was partitioned between water and ethyl acetate (3×). The organics were dried over magnesium sulfate, filtered and solvent removed under reduced pressure. The resultant oil was purified via silica gel chromatography (10-20% ethyl acetate/hexanes) to afford the title compound (1.53 g., 44%). MS (ESI) m/z 337 [M+1]⁺.

D. N-{2-[(tert-butoxy)carbonylamino]ethyl}-5-phenylpentanamide. To a solution of 5-phenylvaleric acid (2.5 g., 14.02 mmol) in dimethyl formamide (30 mL) was added EDCI (2.94 g., 15.42 mmol) and hydroxybenzotriazole (2.08 g., 15.42 mmol). The solution was allowed to stir for 15 minutes at ambient temperature. N-Boc-ethylenediamine (2.46 g., 15.42 mmol) in dimethyl formamide (2 mL) was added and stirring continued overnight at ambient temperature. The solution was condensed under reduced pressure and partitioned between 1N NaOH and ethyl acetate (3×). The organics were combined, dried over magnesium sulfate, filtered and solvent removed under reduced pressure. The resultant oil was purified via silica gel chromatography (50-60% ethyl acetate/hexanes) to afford the title compound (3.29 g., 73%). MS (ESI) m/z 321 [M+1]⁺.

### 5.1.49 SYNTHESIS OF 2-(5-(6-ISOQUINOLYL)(1,3,4-THIADIAZOL-2-YL))-1-PHENYLPROP-2-YLAMINE

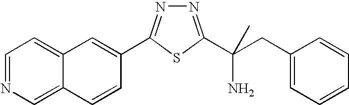

A. 2-(5-(6-Isoquinolyl)(1,3,4-thiadiazol-2-yl))-1-phenylprop-2-ylamine HCl. N-({2-[(tert-Butoxy)carbonylamino]-2-methyl-3-phenyl-1-thioxopropyl}amino)-6-isoquinolylcarboxamide (190 mg, 0.4 mmol was dissolved in THF (10 mL), Lawesson's Reagent (343 mg, 0.8 mmol) was added and then heated to 65° C. in a pressure tube for 3 days. The reaction was concentrated and then purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to give an oil. The oil was treated with 4N HCl-dioxane for 2 hours to remove the Boc group. The product was concentrated, rinsed with ether, and then filtered to give a white solid as the HCl salt, 97.6% pure, (50 mg, 34% yield). ¹H NMR (400 MHz, DMSO-d₆) δ 9.66 (s, 1H), 9.24 (bs, 3H), 8.80 (s, 1H), 8.69 (d, J=6.0 Hz, 1H), 8.48 (d, J=8.8 Hz, 1H), 8.39 (d, J=8.8 Hz, 1H), 8.26 (d, J=6.0 Hz, 1H), 7.29 (m, 3H), 7.10 (m, 2H), 3.47 (dd, J=12.8 Hz, 1H), 1.86 (s, 3H). MS (ESI) m/z 347.1 [M+1]⁺.

B. N-({2-[(tert-Butoxy)carbonylamino]-2-methyl-3-phenyl-1-thioxopropyl}amino)-6-isoquinolylcarboxamide. Isoquinoline-6-carbohydrazide (100 mg, 0.5 mmol), 2-[(tert-Butoxy)carbonylamino]-2-methyl-4-phenylbutanoic acid (148 mg, 0.5 mmol), O-(benzotriazol-1-yl)-N,N,N′,N′-tetramethyluronium hexafluorophosphate (HBTU, 222 mg, 0.6 mmol), diisopropylethylamine (200 μL, 1.6 mmol), and DMF (5 mL) were added together and stirred for 3 hours. The crude reaction was added with dichloromethane and extracted with sat. NaHCO₃, water, and brine in succession. The organic layer was dried over Na₂SO₄, filtered, and solvent removed under reduced pressure to give an oil. The oil was flash chromatographed and eluted with 80% EtOAc-hexane. The product fractions were combined and concentrated down to give a white solid, (190 mg, 80%). MS (ESI) m/z 449.4 [M+1]⁺.

C. 2-[(tert-Butoxy)carbonylamino]-2-methyl-4-phenylbutanoic acid. α-Methyl-DL-phenylalanine (0.5 g, 2.8 mmol), 2-(Boc-oxyimino)-2-phenylacetonitrile (756 mg, 3.1 mmol), triethylamine (0.78 mL, 5.6 mmol), aqueous acetone (1:1, 10 mL), and dimethylformamide (3 drops) were added together and stirred at room temperature for 16 hours. The acetone solvent was removed under reduced pressure and the remaining solution was extracted with EtOAc. The organic layer was extracted with 1N KHSO₄, water, and brine. Then it was dried over MgSO₄, filtered, and concentrated. The residue was flash chromatographed with 30% EtOAc-hexane. The product fractions were combined, concentrated, and then triturated in hexane to give a white solid, (0.5 g, 64% yield). MS (ESI) m/z 280.3 [M+1]⁺.

### 5.1.50 SYNTHESIS OF 1-(5-(6-ISOQUINOLYL)(1,3,4-THIADIAZOL-2-YL))-2-PHENYLETHYLAMINE

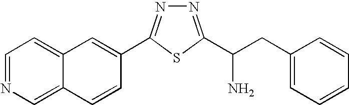

A. 1-(5-(6-Isoquinolyl)(1,3,4-thiadiazol-2-yl))-2-phenylethylamine HCl. N-({2-[(tert-Butoxy)carbonylamino]-3-phenyl-1-thioxopropyl}amino)-6-isoquinolylcarboxamide (180 mg, 0.4 mmol wad dissolved in THF (10 mL), Lawesson's Reagent (335 mg, 0.8 mmol) was added and then heated to 65° C. in a pressure tube for 3 days. The reaction was concentrated and then purified using reverse-phase semi-preparatory HPLC (20-70% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over Na₂SO₄, filtered and solvent removed under reduced pressure to give an oil. The oil was treated with 4N HCl-dioxane for 2 hours to remove the Boc group. The product was concentrated, rinsed with ether, and then filtered to give a white solid with 97.6% pure, (31 mg, 23% yield). ¹H NMR (400 MHz, DMSO-d₆) δ 9.67 (s, 1H), 9.16 (bs, 3H), 8.77 (s, 1H), 8.69 (d, J=6.0 Hz, 1H), 8.46 (d, J=8.8 Hz, 1H), 8.38 (d, J=8.8 Hz, 1H), 8.30 (bs, 1H), 7.30 (m, 5H), 5.46 (bs, 1H), 3.53 (m, 1H), 3.31 (dd, J=10.4, 13.6 Hz, 1H). MS (ESI) m/z 333.4 [M+1]⁺.

B. N-({2-[(tert-Butoxy)carbonylamino]-3-phenyl-1-thioxopropyl}amino)-6-isoquinolyl carboxamide. Isoquinoline-6-carbohydrazide (100 mg, 0.5 mmol), DL N-Boc-phenylalanine (141 mg, 0.5 mmol), O-(benzotriazol-1-yl)-N,N,N′,N′-tetramethyluronium hexafluorophosphate (HBTU, 222 mg, 0.6 mmol), diisopropylethylamine (200 μL, 1.6 mmol), and DMF (5 mL) were added together and stirred for 3 hours. The crude reaction was added with dichloromethane and extracted with sat. NaHCO₃, water, and brine in succession. The organic layer was dried over Na₂SO₄, filtered, and solvent was removed under reduced pressure to give an oil. The oil was flash chromatographed and eluted with 80% EtOAc-hexane. The product fractions were combined and concentrated down to give a white solid, (180 mg, 78%). MS (ESI) m/z 435.3 [M+1]⁺.

### 5.1.51 SYNTHESIS OF 2-(4-(6-ISOQUINOLYL)(1,2,3-TRIAZOLYL))-3-PHENYLPROPYLAMINE

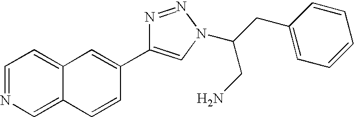

A. 2-(4-(6-isoquinolyl)(1,2,3-triazolyl))-3-phenylpropylamine. A solution of 2-[2-(4-(6-isoquinolyl)(1,2,3-triazolyl))-3-phenylpropyl]benzo[c]azoline-1,3-dione (0.14 g, 0.3 mmol) and hydrazine (0.1 mL, excess) in ethanol (20 mL) were reacted according to General Procedure A. Product was purified using reverse-phase semi-preparatory HPLC (10-40% acetonitrile+0.1% TFA in H₂O+0.1% TFA, over 30 minutes). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.5% purity, (30 mg, 31%). ¹H NMR (300 MHz, d₆-DMSO) δ 9.29 (s, 1H), 8.82 (s, 1H), 8.50 (d, J=5.77, 1H), 8.40 (s, 1H), 7.86 (ABq, J_{AB}=8.24, 2H), 7.24-7.12 (overlapping m, 5H), 4.81 (m, 1H), 3.19 (m 2H), 3.08 (d, J=6.0, 2H); MS (ESI) m/z 330.4 [M+1]⁺.

B. 2-[2-(4-(6-isoquinolyl)(1,2,3-triazolyl))-3-phenylpropyl]benzo[c]azoline-1,3-dione. A solution of 2-[3-diazo-2-benzyl-3-azaprop-3-enyl]benzo[c]azoline-1,3-dione (0.2 g, 0.65 mmol) and 6-ethynylisoquinoline (0.1 g, 0.65 mmol) in 1:1 t-butanol:water (5 mL) was treated with ascorbic acid (0.1 mL, 1M in water), followed by copper sulfate-penta hydrate (2 mg, 0.008 mmol) in water (0.05 mL). After stirring at room temperature overnight, the reaction was poured into water (50 mL), cooled on ice and the resulting solid removed by filtration. The solid was further washed with water and then taken up in CH₂Cl₂, washed with water, and saturated sodium chloride in succession, dried over magnesium sulfate, filtered and solvent removed under reduced pressure. Purification using silica gel flash column chromatography (1:1 EtoAc:hexanes to 5% methanol in EtOAc) provided the title compound, (0.140 g, 47%). MS (ESI) m/z 460.0 [M+1]⁺.

C. 2-[3-diazo-2-benzyl-3-azaprop-3-enyl]benzo[c]azoline-1,3-dione. A solution of 2-(2-hydroxy-3-phenylpropyl)benzo[c]azoline-1,3-dione (0.5 g, 1.8 mmol) in CH₂Cl₂ (10 mL) was treated with triethyl amine (0.5 mL, 3.6 mmol) and methanesulfonyl chloride (0.3 mL, 3.9 mmol). The reaction was stirred at room temperature 30 minutes and additional triethyl amine (0.2 mL) and methanesulfonyl chloride (0.1 mL) were added and the reaction stirred an additional 20 minutes. Excess solvent and reagents were removed under reduced pressure. The intermediate mesylate was taken up in DMSO (10 mL), sodium azide (0.5 g, 7.7 mmol) was added, and the reaction was heated to 130° C. for 480 seconds in a microwave reactor. The solution was poured into water and product was extracted with CH₂Cl₂. Organic fractions were combined and washed with water and saturated sodium chloride, dried over magnesium sulfate, filtered, and solvent was removed under reduced pressure to afford crude product. Purification using Biotage column chromatography (10-50% EtOAc in hexanes) provided the title compound (0.187 g, 35%). MS (ESI) m/z 307.0 [M+1]⁺.

D. 2-(2-hydroxy-3-phenylpropyl)benzo[c]azoline-1,3-dione. A solution of 2-benzyloxirane (1.0 g, 7.5 mmol), phthalimide (1.1 g, 7.5 mmol), and pyridine (0.05 mL) were combined in n-butanol (5 mL) and heated on a 120° C. sand bath overnight. Excess solvent was removed under reduced pressure to afford crude product. Purification using Biotage column chromatography (25-0% hexanes in CH₂Cl₂) provided the title compound (1.39 g, 66%). MS (ESI) m/z 282.1 [M+1]⁺.

### 5.1.52 SYNTHESIS OF 3-(5-(6-ISOQUINOLYL)(1,3,4-THIADIAZOL-2-YL))-4-PHENYLBUTYLAMINE

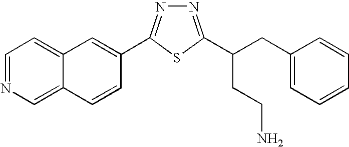

A. 3-(5-(6-Isoquinolyl)(1,3,4-thiadiazol-2-yl))-4-phenylbutylamine HCl. N-({4-[(tert-Butoxy)carbonylamino]-2-benzyl-1-thioxobutyl}amino)-6-isoquinolylcarboxamide (50 mg, 0.1 mmol) was dissolved in THF (0.5 mL), Lawesson's Reagent (87 mg, 0.2 mmol) was added, and the mixture was heated in a microwave (100° C., 10 min.). The reaction was flash chromatographed with 60% EtOAc-hexane to give a slightly yellow solid. The oil was treated with 4N HCl-dioxane for 2 hours to remove the Boc group. The product was concentrated, rinsed with ether, and then filtered to give a white solid, 98.2% pure, (29 mg, 74%). ¹H NMR (400 MHz, CD₃OD) δ 9.68 (s, 1H), 8.73 (s, 1H), 8.69 (d, J=6.0 Hz, 1H), 8.47 (d, J=8.8 Hz, 1H), 8.38 (dd, J=8.8, 1.6 Hz, 1H), 8.29 (d, J=6.0 Hz, 1H), 7.92 (bs, 3H), 7.24 (m, 5H), 3.96 (m, 1H), 3.19 (dd, J=13.6, 6.8 Hz, 1H), 3.09 (dd, J=14.4, 8.4 Hz, 1H), 2.85 (m, 2H), 2.14 (m, 2H). MS (ESI) m/z 344.3 [M+1]⁺.

B. N-({4-[(tert-Butoxy)carbonylamino]-2-benzyl-1-thioxobutyl}amino)-6-isoquinolyl carboxamide. Isoquinoline-6-carbohydrazide (70 mg, 0.4 mmol), racemic 4-[(tert-butoxy)carbonylamino]-2-benzylbutanoic acid (111 mg, 0.5 mmol), O-(benzotriazol-1-yl)-N,N,N′,N′-tetramethyluronium hexafluorophosphate (HBTU, 151 mg, 0.4 mmol), diisopropylethylamine (25 μL, 2 mmol), and DMF (2 mL) were added together and stirred for 3 hours. The crude reaction was treated with dichloromethane and extracted with sat. NaHCO₃, water, and brine in succession. The organic layer was dried over Na₂SO₄, filtered, and solvent was removed under reduced pressure to give an oil. The oil was flash chromatographed and eluted with 80% EtOAc-hexane. The product fractions were combined and concentrated down to give a white solid, (80 mg, 60%). MS (ESI) m/z 464.5 [M+1]⁺.

### 5.1.53 SYNTHESIS OF 2-(6-ISOQUINOLYL)-5-[4-BENZYL(4-PIPERIDYL)]-1,3,4-THIADIAZOLE

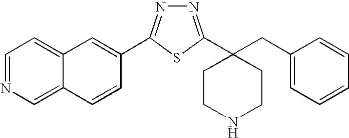

A. 2-(6-Isoquinolyl)-5-[4-benzyl(4-piperidyl)]-1,3,4-thiadiazole HCl. N-({4-[(tert-Butoxy)carbonylamino]-2-benzyl-1-thioxobutyl}amino)-6-isoquinolylcarboxamide (340 mg, 0.7 mmol) was dissolved in THF (3 mL), Lawesson's Reagent (580 mg, 1.4 mmol) was added, and the mixture was heated in a microwave (60° C., 10 min.). The reaction was flash chromatographed with 60% EtOAc-hexane to give an oil. The oil was dissolved in MeOH (5 mL), treated with 4N HCl-dioxane, and then stirred for 2 hours to remove the Boc group. The product was concentrated, rinsed with ether, and then filtered to give a white solid as the HCl salt with 95.9% purity. ¹H NMR (400 MHz, CD₃OD) δ 9.84 (s, 1H), 8.89 (s, 1H), 8.62 (m, 4H), 7.19 (m, 3H), 6.93 (m, 2H), 3.44 (bd, J=12.8 Hz, 2H), 3.17 (s, 2H), 3.07 (t, J=10.4 Hz, 2H), 2.61 (bd, J=14.8 Hz, 2H), 2.26 (td, J=14.0, 4.0 Hz, 2H). MS (ESI) m/z 378.4 [M+1]⁺.

B. tert-Butyl 4-{[(6-isoquinolylcarbonylamino)amino]thioxomethyl}-4-benzylpiperidine carboxylate. Isoquinoline-6-carbohydrazide (188 mg, 1 mmol), 1-[(tert-butyl)oxycarbonyl]-4-benzylpiperidine-4-carboxylic acid (319 mg, 1 mmol), O-(benzotriazol-1-yl)-N,N,N′,N′-tetramethyluronium hexafluorophosphate (HBTU, 379 mg, 1 mmol), triethylamine (0.5 mL, 5 mmol), and DMF (3 mL) were added together and stirred for 3 hours. The crude reaction was treated with dichloromethane and extracted with sat. NaHCO₃, water, and brine in succession. The organic layer was dried over Na₂SO₄, filtered, and solvent removed under reduced pressure to give an oil. The oil was flash chromatographed with 80% EtOAc-hexane. The product fractions were combined and concentrated down to give a white solid, (340 mg, 70%). MS (ESI) m/z 464.5 [M+1]⁺.

C. 1-[(tert-Butyl)oxycarbonyl]-4-benzylpiperidine-4-carboxylic acid. Ethyl isonipecotate (5 g, 32 mmol), di-tert-butyl dicarbonate (7.6 g, 35 mmol), triethylamine (6 mL), and MeOH were added together at 0° C., and then stirred for 16 hours. The reaction was concentrated under reduced pressure to give an oil. Then the oil (1 g, 3.9 mmol) was taken up in THF (3 mL) followed by the addition of 1M lithium bis(trimethylsilyl)amide (3.9 mL, 3.9 mmol) in THF via a syringe at −78° C. The reaction was allowed to warm to room temperature and stirred for 1 hour. The reaction was cooled to −78° C. again using a dry-ice bath, followed by addition of benzyl bromide (0.55 mL, 3.9 mmol) in THF (3 mL). Then the dry-ice bath was removed allowing the reaction to warm to room temperature and the reaction was stirred for 1 hour. The reaction was extracted with saturated ammonium chloride and EtOAc. The organic layer was concentrated under reduced pressure to give an oil. The benzylated ester was hydrolyzed by addition of NaOH (1.6 g, 40 mmol) in MeOH (40 mL) and then heated in the microwave (100° C., 10 min). The reaction was concentrated under reduced pressure and then extracted with EtOAc and water. The organic layer containing the product was extracted with 1N HCl, water, and brine in succession. The organic layer was dried over MgSO₄, filtered, and concentrated to give a white solid, (0.77 g, 64% yield). MS (ESI) m/z 320.1 [M+1]⁺.

### 5.1.54 SYNTHESIS OF 3-(4-(6-ISOQUINOLYL)(1,2,3-TRIAZOLYL))-2-BENZYLPROPYLAMINE HYDROCHLORIDE

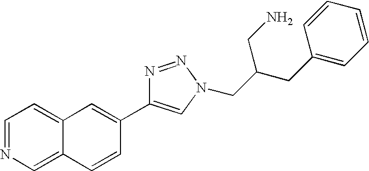

A. 3-(4-(6-isoquinolyl)(1,2,3-triazolyl))-2-benzylpropylamine hydrochloride. A solution of (tert-butoxy)-N-[3-(4-(6-isoquinolyl)(1,2,3-triazolyl))-2-benzylpropyl]carboxamide (65 mg, 0.15 mmol) in CH₂Cl₂/MeOH (˜4.0 mL) was treated with HCl (2.0 mL, excess, 1 M in Et₂O). The reaction was stirred at room temperature until starting material was completely consumed. The solution was concentrated under reduced pressure to afford the product as an HCl salt, 99.5% purity, (45 mg, 89%). ¹H NMR (300 MHz, CD₃OD) δ 9.68 (bs, 1H), 8.75 (s, 1H), 8.71 (s, 1H), 8.55-8.43 (overlapping m, 4H), 7.36-7.21 (overlapping m, 5H), 4.63 (dABq, J=5.63, J_{AB}=8.24, 2H), 3.07 (m 2H), 2.87 (m, 2H), 2.75 (m, 1H); MS (ESI) m/z 344.1 [M+1]⁺.

B. (tert-butoxy)-N-[3-(4-(6-isoquinolyl)(1,2,3-triazolyl))-2-benzylpropyl]carboxamide. A solution of N-[3-azido-2-benzylpropyl](tert-butoxy)carboxamide (0.28 g, 0.96 mmol) and 6-ethynylisoquinoline (0.15 g, 0.98 mmol) in 1:1 t-butanol:water (10 mL) was treated with sodium ascorbate (0.1 mL, 1 M in water), followed by copper sulfate-penta hydrate (2.4 mg, 0.01 mmol) in water (0.12 mL). After stirring at room temperature overnight, the reaction was poured into water (50 mL), cooled on ice and the resulting solid removed by filtration. The solid was further washed with water and then taken up in CH₂Cl₂, washed with water and saturated sodium chloride in succession, dried over magnesium sulfate, filtered, and solvent was removed under reduced pressure. Purification using silica gel flash column chromatography (10% methanol in CH₂Cl₂) provided the title compound, (0.069 g, 17%). MS (ESI) m/z 444.3 [M+1]⁺.

C. N-[3-azido-2-benzylpropyl](tert-butoxy)carboxamide. A solution of (tert-butoxy)-N-[3-hydroxy-2-benzylpropyl]carboxamide (0.56 g, 2.1 mmol) in CH₂Cl₂ (15 mL) was treated with triethyl amine (0.38 mL, 2.7 mmol) and methanesulfonyl chloride (0.21 mL, 2.7 mmol). The reaction was stirred at room temperature 60 minutes. Excess solvent and reagents were removed under reduced pressure. The intermediate mesylate was taken up in DMF (15 mL), sodium azide (0.41 g, 6.3 mmol) was added and the reaction was heated on a 65° C. oil bath overnight. DMF was removed under reduced pressure. Purification using Biotage column chromatography (10-60% EtOAc in hexanes) provided the title compound (0.39 g, 63%). MS (ESI) m/z 291.0 [M+1]⁺.

D. (tert-butoxy)-N-[3-hydroxy-2-benzylpropyl]carboxamide. A solution of ethyl 3-[(tert-butoxy)carbonylamino]-2-benzylpropanoate (1.4 g, 4.6 mmol) and lithium aluminum hydride (0.52 g, 13.8 mmol) in THF (50 mL) were reacted at room temperature overnight according to General Procedure F to provide crude material. Purification using Biotage column chromatography (0-40% methanol in CH₂Cl₂) provided the title compound (0.58 g, 50%). MS (ESI) m/z 266.0 [M+1]⁺.

### 5.1.55 SYNTHESIS OF [(3R,4R)-1,4-BISBENZYL(3-PIPERIDYL)](5-(6-ISOQUINOLYL)(1,3,4-THIADIAZOL-2-YL))AMINE

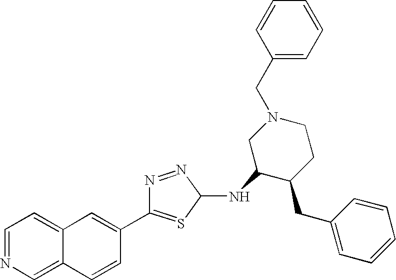

A. [(3R,4R)-1,4-bisbenzyl(3-piperidyl)] (5-(6-isoquinolyl)(1,3,4-thiadiazol-2-yl))amine. N-[({[(3R,4R)-1,4-bisbenzyl(3-piperidyl)]amino}thioxomethyl)amino]-6-isoquinolylcarboxamide (105 mg, 0.206 mmol) following General Procedure A affords a crude residue of product. The sample was dissolved in 1:1 DMSO:methanol and purified using reverse phase HPLC on a C18 column with 10-70% acetonitrile/H₂O/0.1% TFA to provide the title compound (20 mg, 20%). ¹H NMR (400 MHz, CD₃OD) δ 9.25 (s, 1H), 8.47 (d, J=5.6, 1H), 8.25 (s, 1H); 8.10 (m, 2H), 7.9 (d, J=6.0, 5H), 7.46 (m, 5H), 7.22 (m, 5H), 4.36 (m, 1H); 4.24 (m, 1H); 4.04 (brd s, 1H); 3.81 (brd s, 1H); 3.51 (brd s, 1H); 3.10 (brd m, 2H); 2.79 (brd m, 2H); 2.30 (brd s, 1H); 2.03-1.88 (m, 2H); MS (ESI) m/z 492[M+1]⁺.

B. N-[({[(3R,4R)-1,4-bisbenzyl(3-piperidyl)]amino}thioxomethyl)amino]-6-isoquinolyl carboxamide. Isoquinoline-6-carbohydrazide (70.3 mg, 0.375 mmol) was reacted with (3R,4R)-1,4-bisbenzyl-3-piperidylamine (100.0 mg, 0.357 mmol) according to General Procedure D to afford a crude residue of title compound. No purification was performed and the crude material was used in next step. MS (ESI) m/z 510[M+1]⁺.

C. (3S,4R)-4-benzyl-3-piperidylamine. (3R,4R)-1,4-Bisbenzyl-3-piperidylamine (100 mg, 0.356 mmol) was added to 4 mL of methanol and 10% Pd/C (100 mg, 1:1, wt.). All oxygen was evacuated from the reaction vessel and replaced with hydrogen at 1 atm. The reaction was stirred for 18 hours and then filtered and evaporated. The resulting residue was placed in 5 mL HCl/Dioxane and stirred for 30 min. ¹H NMR (trans) (400 MHz, CD₃OD) δ 7.25 (m, 5H), 3.67-3.635 (dd, J=12, 3.6, 1H), 3.45-3.38 (ddd, J=12, 3.6, 1H), 3.35-3.29 (m, 1H), 3.20-3.17 (dd, J=13.4, 3.6, 1H), 3.09-3.03 (t, J=12, 1H), 2.92-2.85 (m, 1H), 1.52-1.41 (m, 1H); MS (ESI) m/z 264[M+1]⁺.

D. (3R,4R)-1,4-bisbenzyl-3-piperidylamine. 1-Benzyl-4-(phenylmethylene)-3-piperidyl hydrazine (2.26 g, 7.74 mmol) was added to 100 mL of methanol followed by the addition of Raney Nickel:H₂O slurry 3 mL. The reaction was placed in a Parr Hydrogenator at 60 psi for 4 days. The reaction was filtered and concentrated. Residue was purified using flash chromatography CH₂Cl₂:methanol (9:1) to give two diastereomers, cis and trans. MS (ESI) m/z 282[M+1]⁺.

E. 1-Benzyl-4-(phenylmethylene)-3-piperidylhydrazine. To a slurry of triphenylbenzyl phosphine chloride (4.03 g, 10.4 mmol) in THF was added potassium tertbutoxide and the mixture was stirred for 30 min. A solution of (tert-butoxy)-N-{(tert-butoxy)-N-[4-oxo-1-benzyl(3-piperidyl)]carbonylamino}carboxamide (2.9 g, 6.91 mmol) in THF was added and the reaction was stirred at room temperature for 20 hours. The reaction was then quenched with 1M HCl to pH 7.0 and concentrated. Residue was extracted with ethyl acetate and the organic layers collected and dried over MgSO₄. The resulting material was purified using flash chromatography (hexane:ethyl acetate 8:2) to give 2.6 g white solid, 76%. MS (ESI) m/z 494[M+1]⁺. The solid was treated with CH₂Cl₂:TFA (1:1) 10 mL and stirred for 3 hours. The reaction was concentrated to give 2.26 g white solid as the trifluoroacetate salt. MS (ESI) m/z 293[M+1]⁺.

F. (tert-butoxy)-N-{(tert-butoxy)-N-[4-oxo-1-benzyl(3-piperidyl)]carbonylamino}carboxamide 1-Benzylpiperidin-4-one (5.0 g, 26.4 mmol) was added to 50 mL THF and cooled to −78° C. LDA was added drop wise and the reaction was stirred for 1 hour. A solution of DBAD and THF was added dropwise and the reaction was stirred for 2 hours. After 2 hours, the reaction was quenched with acetic acid (3.74 mL, 66.0 mmol). The reaction was then extracted with ethyl acetate, the organic layers were collected and dried over MgSO₄. The resulting material was purified using flash chromatography (hexane:ethyl acetate 8:2) to give a yellow oil, (10 g, 90%). MS (ESI) m/z 420[M+1]⁺.

### 5.1.56 [2-AMINO-1-BENZYLETHYL](5-PYRIDINO[3,4-B]PYRIDIN-3-YL(1,3,4-THIADIAZOL-2-YL))AMINE

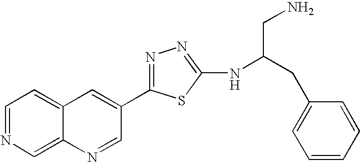

A. [2-amino-1-benzylethyl] (5-pyridino[3,4-b]pyridin-3-yl(1,3,4-thiadiazol-2-yl))amine. 2-{3-phenyl-2-[(5-pyridino[3,4-b]pyridin-3-yl(1,3,4-thiadiazol-2-yl))amino]propyl}benzo[c]azolidine-1,3-dione (211 mg, 0.428 mmol), following General Procedure A to afford a crude residue of product. Sample was dissolved in 1:1 DMSO:methanol and purified using RP-HPLC on a C18 column with 10-70% acetonitrile/H₂O/0.1% TFA to provide the title compound (41 mg, 26%). ¹H NMR (400 MHz, CDCl₃) δ 9.54 (d, 2H, J=2.8), 8.68 (d, 1H, J=5.6), 8.37 (d, 1H, J=2.4), 7.70 (d, 1H, J=5.6), 7.33-7.25 (m, 5H), 3.96 (m, 1H), 3.15 (dd, J=6.4 and 13.6), 2.96 (m, 3H); MS (ESI) m/z 363[M+1]⁺.

B. 2-{3-phenyl-2-[(5-pyridino[3,4-b]pyridin-3-yl(1,3,4-thiadiazol-2-yl))amino]propyl}benzo[c]azolidine-1,3-dione. N-[({[2-(1,3-dioxobenzo[c]azolidin-2-yl)-1-benzylethyl]amino}thioxo methyl)amino]pyridino[3,4-b]pyridin-3-ylcarboxamide (282 mg, crude, 1.14 mmol) was reacted according to General Procedure B. No purification was performed and the crude material was used in next step. MS (ESI) m/z 493[M+1]⁺.

C. N-[({[2-(1,3-dioxobenzo[c]azolidin-2-yl)-1-benzylethyl]amino}thioxomethyl)amino]pyridino[3,4-b]pyridin-3-ylcarboxamide. Pyridino[3,4-b]pyridine-3-carbohydrazide (215 mg, 1.14 mmol) was reacted with 2-(2-amino-3-phenylpropyl)benzo[c]azolidine-1,3-dione (332.8 mg, 1.18 mmol) were reacted according to General Procedure D to afford a crude residue of the title compound. No purification was performed and the crude material was used in next step. MS (ESI) m/z 511[M+1]⁺.

### 5.1.57 SYNTHESIS OF 2-AMINO-1-BENZYLETHYL-[5-(8-BROMOPYRIDINO[3,4-e]PYRIDIN-2-YL)(1,3,4-THIADIAZOL-2-YL)]AMINE

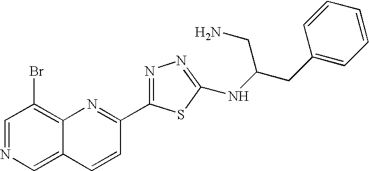

A. 2-Amino-1-benzylethyl-[5-(8-bromopyridino[3,4-e]pyridin-2-yl)(1,3,4-thiadiazol-2-yl)]amine. A solution of 2-(2-{[5-(8-bromopyridino[3,4-e]pyridin-2-yl)(1,3,4-thiadiazol-2-yl)]amino}-3-phenylpropyl)benzo[c]azolidine-1,3-dione (0.17 g, 0.29 mmol) and hydrazine (0.09 mL, excess) in ethanol (5 mL) were reacted according to General Procedure A. Purification was performed using reverse-phase semi-preparatory HPLC (10-40% acetonitrile+0.1% TFA in H₂O+0.1% TFA, over 30 min). Fractions containing clean product were neutralized with potassium carbonate and solvent was removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 97.0% purity, (32 mg, 24%). ¹H NMR (400 MHz, DMSO-d₆) δ 9.37 (d, J=1.2, 1H); 9.02 (d, J=1.2, 1H); 8.69 (dd, J=8.8, 1.2, 1H); 8.39 (dd, J=8.8, 1.2, 1H); 7.29-7.28 (m, 5H); 7.20-7.17 (m, 2H); 3.92-3.89 (m, 1H); 2.97 (dd, J=12.8, 6.4, 1H); 2.85 (dd, J=12.8, 6.4, 1H); 2.77-2.68 (m, 2H). MS (ESI) m/z 443.5 [M+1]⁺.

B. 2-(2-{[5-(8-Bromopyridino[3,4-e]pyridin-2-yl)(1,3,4-thiadiazol-2-yl)]amino}-3-phenyl propyl)benzo[c]azolidine-1,3-dione. Methane sulfonic acid (1.0 mL, excess) and N-[({[2-(1,3-dioxobenzo[c]azolidin-2-yl)-1-benzylethyl]amino}thioxomethyl)amino](8-bromopyridino[3,4-e]pyridin-2-yl)carboxamide (0.37 g, 0.62 mmol) were reacted as described in General Procedure B to provide pure material (0.36 g, 47%). MS (ESI) m/z 573.3 [M+1]⁺.

C. N-[({[2-(1,3-dioxobenzo[c]azolidin-2-yl)-1-benzylethyl]amino}thioxomethyl)amino](8-bromopyridino[3,4-e]pyridin-2-yl)carboxamide. The hydrochloride salt of 2-(2-amino-3-phenylpropyl)benzo[c]azolidine-1,3-dione (0.29 g, 1.0 mmol), triethylamine (0.15 mL, 1.0 mmol), di(2-pyridyloxy)methane-1-thione (0.24 g, 1.0 mmol), and 8-bromopyridino[3,2-c]pyridine-2-carbohydrazide (0.27 g, 1.0 mmol) were reacted according to General Procedure C. The residual solid was purified using flash column chromatography on silica gel with 100% hexanes to provide the desired product (0.37 g, 54%). MS (ESI) m/z 591.2 [M+1]⁺.

### 5.1.58 SYNTHESIS OF [(1R)-2-AMINO-1-BENZYLETHYL][5-(8-BROMOPYRIDINO[3,4-e]PYRIDIN-2-YL)(1,3,4-THIADIAZOL-2-YL)]AMINE

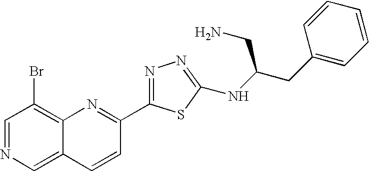

A. [(1R)-2-amino-1-benzylethyl][5-(8-bromopyridino[3,4-e]pyridin-2-yl)(1,3,4-thia-diazol-2-yl)]amine. A solution of 2-((2R)-2-{[5-(8-bromopyridino[3,4-e]pyridin-2-yl)(1,3,4-thiadiazol-2-yl)]amino}-3-phenylpropyl)benzo[c]azolidine-1,3-dione (0.1 g, 0.17 mmol) and hydrazine (0.3 mL, excess) in ethanol (10 mL) were reacted according to General Procedure A. Purification was performed using reverse-phase semi-preparatory HPLC (10-40% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 97.7% purity, (60 mg, 77%). ¹H NMR (400 MHz, DMSO-d₆) δ 9.37 (d, J=1.2, 1H); 9.02 (d, J=1.2, 1H); 8.69 (dd, J=8.8, 1.2, 1H); 8.39 (dd, J=8.8, 1.2, 1H); 7.29-7.28 (m, 5H); 7.20-7.17 (m, 2H); 3.92-3.89 (m, 1H); 2.97 (dd, J=12.8, 6.4, 1H); 2.85 (dd, J=12.8, 6.4, 1H); 2.77-2.68 (m, 2H). (ESI) m/z 443.5 [M+1]⁺.

B. 2-((2R)-2-{[5-(8-Bromopyridino[3,4-e]pyridin-2-yl)(1,3,4-thiadiazol-2-yl)]amino}-3-phenylpropyl)benzo[c]azolidine-1,3-dione. Methane sulfonic acid (4.5 mL, excess) and N-[({[(1R)-2-(1,3-dioxobenzo[c]azolidin-2-yl)-1-benzylethyl]amino}thioxomethyl)amino](8-bromopyridino[3,4-e]pyridin-2-yl)carboxamide (1.3 g, 2.2 mmol) were reacted as described in General Procedure B to provide pure material (1.1 g, 88%). MS (ESI) m/z 573.3 [M+1]⁺.

C. N-[({[(1R)-2-(1,3-Dioxobenzo[c]azolidin-2-yl)-1-benzylethyl]amino}thioxomethyl)amino](8-bromopyridino[3,4-e]pyridin-2-yl)carboxamide. The hydrochloride salt of 2-((2R)-2-amino-3-phenylpropyl)benzo[c]azolidine-1,3-dione (1.8 g, 6.4 mmol), triethylamine (0.9 mL, 6.4 mmol), di(2-pyridyloxy)methane-1-thione (1.4 g, 6.4 mmol), and 8-bromopyridino[3,2-c]pyridine-2-carbohydrazide (1.7 g, 6.4 mmol) were reacted according to General Procedure C. The residual solid was purified using flash column chromatography on silica gel with 100% EtOAc to provide the desired product (1.2 g, 31%). MS (ESI) m/z 591.2 [M+1]⁺.

D. 8-bromopyridino[3,2-c]pyridine-2-carbohydrazide. Methyl 8-bromopyridino[3,4-e]pyridine-2-carboxylate (2.0 g, 7.4 mmol) and hydrazine (0.70 mL, 22.5 mmol) in ethanol (15 mL) were reacted as described in General Procedure H. The resulting product was dried under high vacuum to provide the title compound as an off white solid (2.0 g, 100%). MS (ESI) m/z 269.2 [M+1]⁺.

E. Methyl 8-bromopyridino[3,4-e]pyridine-2-carboxylate. A solution of 8-bromopyridino[4,3-b]pyridine-2-carboxylic acid (4 g, 15.8 mmol) and hydrochloric acid (1 mL) in methanol (15 mL) were reacted according to General Procedure I. The resulting product was dried under vacuum to provide the title compound (4.2 g, 100%). MS (ESI) m/z 269.2[M+1]⁺.

### 5.1.59 SYNTHESIS OF [(1R)-2-AMINO-1-BENZYLETHYL][5-(8-ETHYLPYRIDINO[3,4-e]PYRIDIN-2-YL)(1,3,4-THIADIAZOL-2-YL)]AMINE

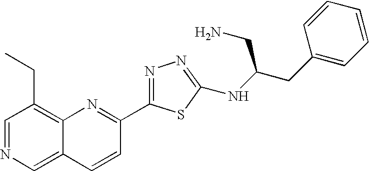

A. [(1R)-2-Amino-1-benzylethyl][5-(8-ethylpyridino[3,4-e]pyridin-2-yl)(1,3,4-thiadiazol-2-yl)l amine. A solution of 2-((2R)-3-phenyl-2-{[5-(8-vinylpyridino[3,4-e]pyridin-2-yl)(1,3,4-thiadiazol-2-yl)]amino}propyl)benzo[c]azolidine-1,3-dione (0.08 g, 0.16 mmol) and hydrazine (0.05 mL, excess) in ethanol (2 mL) were reacted according to General Procedure A. Purification was performed using reverse-phase semi-preparatory HPLC (20-80% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.9% purity, (15 mg, 24%). ¹H NMR (400 MHz, DMSO-d₆) δ 9.58 (s, 1H); 8.82-8.76 (m, 2H); 8.45 (d, J=8.4, 1H); 8.15 (bs, 2H); 7.31-7.30 (m, 5H); 7.21-7.20 (m, 1H); 4.13-4.12 (m, 1H); 3.22-3.16 (m, 2H); 3.10-3.06 (m, 2H); 3.01-2.98 (m, 2H); 1.37 (t, J=7.6, 3H). MS (ESI) m/z 391.6 [M+1]⁺.

B. 2-((2R)-3-phenyl-2-{[5-(8-vinylpyridino[3,4-e]pyridin-2-yl)(1,3,4-thiadiazol-2-yl)]amino}propyl)benzo[c]azolidine-1,3-dione. In a sealed tube, a solution of 2-((2R)-2-{[5-(8-Bromopyridino[3,4-e]pyridin-2-yl)(1,3,4-thiadiazol-2-yl)]amino}-3-phenylpropyl)benzo[c]azo-lidine-1,3-dione (0.23 g, 0.41 mmol) in anhydrous N,N-dimethylformamide was treated with tributylvinyltin (0.17 g, 0.53 mmol) and Pd(PPh₃)₂Cl₂ (17 mg, 0.024 mmol) for 1 hour at 100° C. After completion of the reaction, water was added and the reaction mixture was extracted with ethyl acetate. The pooled organic fractions were washed with water and brine, dried over magnesium sulfate, filtered and the volatiles were removed under reduced pressure. The residual oil was purified using flash chromatography on silica gel (25:75 EtOAc/hexanes) to afford the desired product (0.21 g, 99%). MS (ESI) m/z 519.4 [M+1]⁺.

### 5.1.60 SYNTHESIS OF [(1R)-3-AMINO-1-BENZYLPROPYL](5-(6-ISOQUINOLYL)(1,3,4-THIADIAZOL-2-YL))AMINE

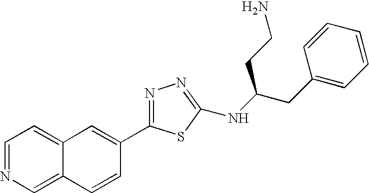

A. [(1R)-3-Amino-1-benzylpropyl](5-(6-isoquinolyl)(1,3,4-thiadiazol-2-yl))amine. A solution of 2-{(3R)-3-[(5-(6-isoquinolyl)(1,3,4-thiadiazol-2-yl))amino]-4-phenylbutyl}benzo[c]azolidine-1,3-dione (0.48 g, 0.40 mmol) and hydrazine (0.3 mL, excess) in ethanol (5.0 mL) were reacted according to General Procedure A. Purification was performed using reverse-phase semi-preparatory HPLC (10-80% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 99.9% purity, (40 mg, 11%). ¹H NMR (400 MHz, DMSO-d₆) δ 9.33 (s, 1H); 8.55 (d, J=5.6, 1H); 8.24 (s, 1H); 8.16 (q, J=9.2, 2H); 7.91 (d, J=5.2, 1H); 7.29-7.26 (m, 5H); 7.19-7.17 (m, 1H); 4.09-4.06 (m, 1H); 2.91-2.87 (m, 2H); 2.71-2.67 (m, 2H); 1.72-1.69 (m, 2H). MS (ESI) m/z 376.5 [M+1]⁺.

B. 2-{(3R)-3-[(5-(6-Isoquinolyl)(1,3,4-thiadiazol-2-yl))amino]-4-phenylbutyl}benzo[c]azo-lidine-1,3-dione. Methane sulfonic acid (5.0 mL, excess) and N-[({[(1R)-3-(1,3-dioxobenzo[c]azolidin-2-yl)-1-benzylpropyl]amino}thioxomethyl)amino]-6-isoquinolylcarbox-amide (0.54 g, 1.03 mmol) were reacted as described in General Procedure B to provide pure material (0.48 g, 92%). MS (ESI) m/z 506.4 [M+1]⁺.

C. N-[({[(1R)-3-(1,3-Dioxobenzo[c]azolidin-2-yl)-1-benzylpropyl]amino}thioxomethyl)amino]-6-isoquinolylcarboxamide. The hydrochloride salt of 2-((3R)-3-amino-4-phenylbutyl)benzo[c]azolidine-1,3-dione (0.53 g, 1.6 mmol), triethylamine (0.22 mL, 1.6 mmol), di(2-pyridyloxy)methane-1-thione (0.37 g, 1.6 mmol), and isoquinoline-6-carbohydrazide (0.30 g, 1.6 mmol) were reacted according to General Procedure C. Purification was performed using Biotage column chromatography (0-100% hexanes in EtOAc) provided clean product (1.15 g, 67%). MS (ESI) m/z 524.2 [M+1]⁺.

D. 2-((3R)-3-Amino-4-phenylbutyl)benzo[c]azolidine-1,3-dione. N-[(1R)-3-(1,3-dioxo benzo[c]azolidin-2-yl)-1-benzylpropyl](tert-butoxy)carboxamide (1.1 g, 2.8 mmol) and hydrochloride (3 mL, 4M in dioxane, 11.2 mmol) in CH₂Cl₂ (100 mL) and methanol (10 mL) were reacted according to General Procedure D. The resulting white solid was dried under high vacuum with heating overnight to give clean product as a hydrochloride salt (0.53 g, 58%). MS (ESI) m/z 295.5 [free base M+1]⁺.

E. N-[(1R)-3-(1,3-Dioxobenzo[c]azolidin-2-yl)-1-benzylpropyl](tert-butoxy)carboxamide. N-[(1R)-3-Hydroxy-1-benzylpropyl](tert-butoxy)carboxamide (0.58 g, 2.18 mmol), resin-bound triphenyl phosphine (1.1 g, 3 mmol P/g resin), phthalimide (0.48 g, 3.28 mmol) and diisopropy-azo-dicarboxylate (0.63 mL, 3.28 mmol) were reacted according to General Procedure E. Purification performed using Biotage column chromatography (0-50% hexanes in EtOAc) provided the desired product (1.1 g, >100%), contaminated with diisopropy-azo-dicarboxylate. MS (ESI) m/z 395.5 [M+1]⁺.

F. N-[(1R)-3-Hydroxy-1-benzylpropyl](tert-butoxy)carboxamide. At −5° C., a solution of (3R)-3-[(tert-butoxy)carbonylamino]-4-phenylbutanoic acid (1 g, 3.57 mmol) in anhydrous tetrahydrofuran (10 mL) was treated with ethyl chloroformate (0.37 mL, 3.93 mmol) and triethylamine (0.55 mL, 3.93 mmol). After 45 min, the cloudy reaction mixture was filtered through celite, and rinsed with THF (10 mL). In a different flask, iodine (0.45 mg, 1.78 mmol) was added to a suspension of sodium borohydride (0.16 g, 4.28 mmol) in dry tetrahydrofuran (15 mL), and the resulting mixture was cooled at 0° C. After 10 min, the filtrate containing the mixed anhydride was transferred via cannula into the sodium borohydride solution. The reaction mixture was allowed to warm to room temperature and stir for 12 hours. Water was then added, and the reaction mixture was extracted with EtOAc, dried over magnesium sulfate, filtered and the volatiles were removed under reduced pressure. The crude oil was purified using chromatography on silica gel (25:75, EtOAc/hexanes) to yield the desired product (0.58 g, 62%). MS (ESI) m/z 266.1 [M+1]⁺.

### 5.1.61 SYNTHESIS OF [(1S)-3-AMINO-1-BENZYLPROPYL](5-(6-ISOQUINOLYL)(1,3,4-THIADIAZOL-2-YL))AMINE

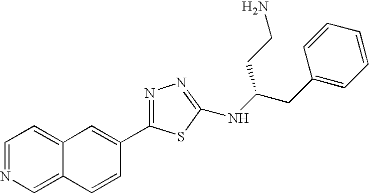

A. [(1S)-3-Amino-1-benzylpropyl](5-(6-isoquinolyl)(1,3,4-thiadiazol-2-yl))amine. A solution of 2-{(3S)-3-[(5-(6-isoquinolyl)(1,3,4-thiadiazol-2-yl))amino]-4-phenylbutyl}benzo[c]azolidine-1,3-dione (0.15 g, 0.29 mmol) and hydrazine (0.1 mL, excess) in ethanol (1.0 mL) were reacted according to General Procedure A. Purification was performed using reverse-phase semi-preparatory HPLC (20-80% acetonitrile+0.1% TFA in H₂O+0.1% TFA, over 30 min). Fractions containing clean product were neutralized with potassium carbonate and solvent was removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 98.1% purity, (98 mg, 89%). ¹H NMR (400 MHz, DMSO-d₆) δ 9.33 (s, 1H); 8.55 (d, J=5.6, 1H); 8.24 (s, 1H); 8.16 (q, J=9.2, 2H); 7.91 (d, J=5.2, 1H); 7.29-7.26 (m, 5H); 7.19-7.17 (m, 1H); 4.09-4.06 (m, 1H); 2.91-2.87 (m, 2H); 2.71-2.67 (m, 2H); 1.72-1.69 (m, 2H). MS (ESI) m/z 376.5[M+1]⁺.

B. 2-{(3S)-3-[(5-(6-Isoquinolyl)(1,3,4-thiadiazol-2-yl))amino]-4-phenylbutyl}benzo[c]azo-lidine-1,3-dione. Methane sulfonic acid (1.5 mL, excess) and N-[({[(1S)-3-(1,3-dioxobenzo[c]azolidin-2-yl)-1-benzylpropyl]amino}thioxomethyl)amino]-6-isoquinolyl carboxamide (0.42 g, 1.03 mmol) were reacted as described in General Procedure B to provide pure material (0.15 g, 37%). MS (ESI) m/z 506.4[M+1]⁺.

C. N-[({[(1S)-3-(1,3-Dioxobenzo[c]azolidin-2-yl)-1-benzylpropyl]amino}thioxomethyl)amino]-6-isoquinolylcarboxamide. The hydrochloride salt of 2-((3S)-3-amino-4-phenylbutyl)benzo[c]azolidine-1,3-dione (0.29 g, 0.87 mmol), triethylamine (0.12 mL, 0.87 mmol), di(2-pyridyloxy)methane-1-thione (0.20 g, 0.87 mmol), and isoquinoline-6-carbohydrazide (0.16 g, 0.87 mmol) were reacted according to General Procedure C. Purification performed using Biotage column chromatography (0-100% hexanes in EtOAc) provided clean product (0.42 g, 92%). MS (ESI) m/z 524.2[M+1]⁺.

D. 2-((3S)-3-Amino-4-phenylbutyl)benzo[c]azolidine-1,3-dione. N-[(1S)-3-(1,3-dioxobenzo[c]azolidin-2-yl)-1-benzylpropyl](tert-butoxy)carboxamide (1.2 g, 3.0 mmol) and HCl (15 mL, 4M in dioxane, excess) in CH₂Cl₂ (10 mL) and methanol (10 mL) were reacted according to General Procedure D. The resulting white solid was dried under high vacuum with heating overnight to give clean product as a hydrochloride salt (0.9 g, 100%). MS (ESI) m/z 295.5[free base M+1]⁺.

E. N-[(1S)-3-(1,3-Dioxobenzo[c]azolidin-2-yl)-1-benzylpropyl](tert-butoxy)carboxamide. N-[(1S)-3-hydroxy-1-benzylpropyl](tert-butoxy)carboxamide (1.3 g, 4.90 mmol), resin-bound triphenyl phosphine (2.45 g, 3 mmol P/g resin), phthalimide (1.08 g, 7.35 mmol) and diisopropy-azo-dicarboxylate (1.42 mL, 7.35 mmol) were reacted according to General Procedure E. Purification performed using Biotage column chromatography (0-50% hexanes in EtOAc) provided the desired product (1.2 g, 63%), contaminated. MS (ESI) m/z 395.5[M+1]⁺.

F. N-[(1S)-3-Hydroxy-1-benzylpropyl](tert-butoxy)carboxamide. At −5° C., a solution of (3S)-3-[(tert-butoxy)carbonylamino]-4-phenylbutanoic acid (2 g, 7.15 mmol) in anhydrous tetrahydrofuran (15 mL) was treated with ethyl chloroformate (0.75 mL, 7.87 mmol) and triethylamine (1.1 mL, 7.87 mmol). After 45 min, the cloudy reaction mixture was filtered through celite, and rinsed with THF (15 mL). In a different flask, iodine (0.91 mg, 3.57 mmol) was added to a suspension of sodium borohydride (0.32 g, 8.58 mmol) in dry tetrahydrofuran (20 mL), and the resulting mixture was cooled at 0° C. After 10 min, the filtrate containing the mixed anhydride was transferred via cannula into the sodium borohydride solution. The reaction mixture was allowed to warm to room temperature and stir for 12 hours. Water was then added, and the reaction mixture was extracted with EtOAc, dried over magnesium sulfate, filtered and the volatiles were removed under reduced pressure. The crude oil was purified using chromatography on silica gel (25:75, EtOAc/hexanes) to yield the desired product (1.3 g, 68%). MS (ESI) m/z 266.1 [M+1]⁺.

### 5.1.62 SYNTHESIS OF 2-(5-{[(1R)-2-AMINO-1-BENZYLETHYL]AMINO}-1,3,4-THIADIAZOL-2-YL)PYRIDINO[3,2-c]PYRIDINE-8-CARBONITRILE

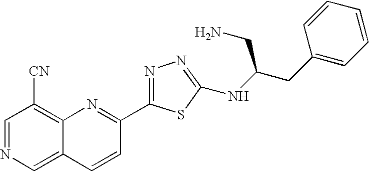

A. 2-(5-{[(1R)-2-Amino-1-benzylethyl]amino}-1,3,4-thiadiazol-2-yl)pyridino[3,2-c]pyridine-8-carbonitrile. A solution of 2-(5-{[(1R)-2-(1,3-dioxobenzo[c]azolidin-2-yl)-1-benzylethyl]amino}-1,3,4-thiadiazol-2-yl)pyridino[3,2-c]pyridine-8-carbonitrile (0.21 g, 0.40 mmol) and hydrazine (0.06 mL, excess) in ethanol (1.5 mL) were reacted according to General Procedure A. Purification was performed using reverse-phase semi-preparatory HPLC (10-80% acetonitrile+0.1% TFA in H₂O+0.1% TFA, over 30 min). Fractions containing clean product were pooled and treated with 1N HCl in ethyl ether to afford the title compound as the hydrochloride salt, 98.5% purity, (92 mg, 59%). ¹H NMR (400 MHz, DMSO-d₆) δ 9.63 (d, J=2, 1H); 9.25 (d, J=2, 1H); 8.76 (dd, J=2, 8.4, 1H); 8.42 (dd, J=2, 8.4, 1H); 8.03 (bs, 2H); 7.26-7.24 (m, 5H); 7.16-7.14 (m, 1H); 3.06-3.03 (m, 2H); 2.95-2.91 (m, 2H). MS (ESI) m/z 388.2 [M+1]⁺.

B. 2-(5-{[(1R)-2-(1,3-Dioxobenzo[c]azolidin-2-yl)-1-benzylethyl]amino}-1,3,4-thiadiazol-2-yl)pyridino[3,2-c]pyridine-8-carbonitrile. In a sealed tube, a solution of 2-((2R)-2-{[5-(8-Bromopyridino[3,4-e]pyridin-2-yl)(1,3,4-thiadiazol-2-yl)]amino}-3-phenylpropyl)benzo[c]azolidine-1,3-dione (0.52 g, 1.9 mmol) in anhydrous N,N-dimethylformamide (5 mL) was treated with copper cyanide (0.24 g, 2.72 mmol) at 140° C. for 12 hours. The reaction mixture was quenched by adding 5 ml of a solution of ammonium hydroxide/ammonium chloride (1:1), allowed to stir for 30 min and extracted with EtOAc. The pooled organic fractions were washed with a solution of ammonium hydroxide/ammonium chloride (1:1) and brine, dried over magnesium sulfate, filtered and the volatiles were removed under reduced pressure. The residual oil was purified using chromatography on silica gel (1:9, EtOAc/hexanes) to yield the clean product (0.46 g, 45%). MS (ESI) m/z 519.4[M+1]⁺.

### 5.1.63 SYNTHESIS OF [(1R)-2-AMINO-1-BENZYLETHYL][5-(8-VINYLPYRIDINO[3,4-e]PYRIDIN-2-YL)(1,3,4-THIADIAZOL-2-YL)]AMINE

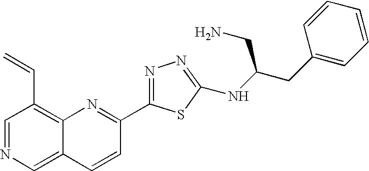

A. [(1R)-2-Amino-1-benzylethyl][5-(8-vinylpyridino[3,4-e]pyridin-2-yl)(1,3,4-thiadiazol-2-yl)l amine. A solution of 2-((2R)-3-phenyl-2-{[5-(8-vinylpyridino[3,4-e]pyridin-2-yl)(1,3,4-thiadiazol-2-yl)]amino}propyl)benzo[c]azolidine-1,3-dione (0.2 g, 0.38 mmol) and methylhydrazine (0.06 mL, excess) in ethanol (4 mL) were reacted according to General Procedure A. Purification was performed using reverse-phase semi-preparatory HPLC (20-60% acetonitrile+0.1% TFA in H₂O+0.1% TFA, over 30 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound, 97.5% pure, (40 mg, 27%). MS (ESI) m/z 389.5 [M+1]⁺.

### 5.1.64 SYNTHESIS OF [(1R)-2-AMINO-1-BENZYLETHYL]{5-[8-(METHYLETHYL)PYRIDINO[3,4-e]PYRIDIN-2-YL](1,3,4-THIADIAZOL-2-YL)}AMINE

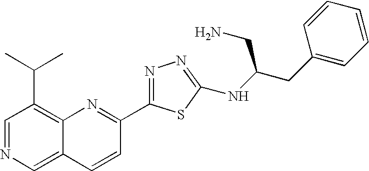

A. [(1R)-2-Amino-1-benzylethyl] (5-[8-(methylethyl)pyridino[3,4-e]pyridin-2-yl](1,3,4-thiadiazol-2-yl))amine. A solution of 2-[(2R)-2-({5-[8-(methylethyl)pyridino[3,4-e]pyridin-2-yl](1,3,4-thiadiazol-2-yl)}amino)-3-phenylpropyl]benzo[c]azolidine-1,3-dione (0.11 g, 0.40 mmol) and hydrazine (0.03 mL, excess) in ethanol (1.0 mL) were reacted according to General Procedure A. Purification was performed using reverse-phase semi-preparatory HPLC (10-100% acetonitrile+0.1% TFA in H₂O+0.1% TFA, over 30 min). Fractions containing clean product were pooled and treated with 1N HCl in ethyl ether to afford the title compound, 98.5% purity, (25 mg, 59%) as the hydrochloride salt. MS (ESI) m/z 405.4[M+1]⁺.

B. 2-[(2R)-2-({5-[8-(Methylethyl)pyridino[3,4-e]pyridin-2-yl](1,3,4-thiadiazol-2-yl)}amino)-3-phenylpropyl]benzo[c]azolidine-1,3-dione. Methane sulfonic acid (2.0 mL, excess) and N-[({[(1R)-2-(1,3-dioxobenzo[c]azolidin-2-yl)-1-benzylethyl]amino}thioxomethyl)amino][8-(methylethyl)pyridino[3,4-e]pyridin-2-yl]carboxamide (0.36 g, 0.65 mmol) were reacted as described in General Procedure B to provide pure material (0.16 g, 46%). MS (ESI) m/z 535.3[M+1]⁺.

C. N-[({[(R)-2-(1,3-Dioxobenzo[c]azolidin-2-yl)-1-benzylethyl]amino}thioxomethyl)amino][8-(methylethyl)pyridino[3,4-e]pyridin-2-yl]carboxamide. The hydrochloride salt of 2-((2R)-2-amino-3-phenylpropyl)benzo[c]azolidine-1,3-dione (0.30 g, 0.96 mmol), triethylamine (0.13 mL, 0.96 mmol), di(2-pyridyloxy)methane-1-thione (0.22 g, 0.96 mmol), and 8-(methylethyl)pyridino[3,2-c]pyridine-2-carbohydrazide (0.32 g, 0.96 mmol) were reacted according to General Procedure C. The residual solid was purified using flash column chromatography on silica gel (100% EtOAc) to provide the desired product (0.36 g, 68%). MS (ESI) m/z 553.5[M+1]⁺.

D. 8-(Methylethyl)pyridino[3,2-c]pyridine-2-carbohydrazide. Methyl 8-(1-methylvinyl)pyridino[3,4-e]pyridine-2-carboxylate (0.22 g, 0.96 mmol) and hydrazine (0.15 mL, 4.81 mmol) in ethanol (1 mL) were reacted as described in General Procedure H. The resulting product was dried under high vacuum to provide the title compound as an off white solid (0.22 g, 100%). MS (ESI) m/z 231.4 [M+1]⁺.

E. Methyl 8-(1-methylvinyl)pyridino[3,4-e]pyridine-2-carboxylate. In a sealed tube, cesium fluoride (0.62 g, 4.11 mmol) was added to a solution of methyl 8-bromopyridino[3,4-e]pyridine-2-carboxylate (0.50 g, 1.87 mmol), 3,3-dibutyl-2-methyl-3-stannahept-1-ene (1.24 g, 3.74 mmol) and palladium bis(tri-tertbutyphosphine) (0.06 g, 0.112 mmol) in anhydrous dioxane (15 mL). The resulting reaction mixture was heated at 100° C. for 1.5 hours, diluted with water, extracted with ethyl acetate, dried over magnesium sulfate, filtered and the volatiles were removed under reduced pressure. The residual oil was purified using chromatography on silica gel (3:7 EtOAc:Hexanes) to afford the clean product (0.22 g, 52%). MS (ESI) m/z 231.4 [M+1]⁺.

F. 3,3-Dibutyl-2-methyl-3-stannahept-1-ene. At −78° C., a solution of isopropenylmagnesium bromide (0.5 M in THF, 18.5 mL, 9.2 mmol) was slowly added to a solution of tributyltin chloride (3.0 g, 9.2 mmol) in anhydrous THF (30 mL). After 1 hour, the reaction was quenched with an aqueous solution of ammonium chloride, extracted with EtOAc, dried over magnesium sulfate, filtered and the volatiles were removed under reduced pressure. The crude oil was used without further purification.

### 5.1.65 SYNTHESIS OF [(1R)-2-AMINO-1-BENZYLETHYL][5-(3-ETHYL(1H-INDAZOL-5-YL))(1,3,4-THIADIAZOL-2-YL)]AMINE

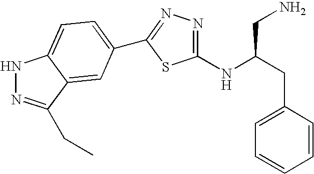

A. [(1R)-2-Amino-1-benzylethyl [5-(3-ethyl(1H-indazol-5-yl))(1,3,4-thiadiazol-2-yl)]amine. A solution of 2-((2R)-2-{[5-(3-ethyl(1H-indazol-5-yl))(1,3,4-thiadiazol-2-yl)]amino}-3-phenylpropyl)benzo[c]azolidine-1,3-dione (0.187 g, 0.367 mmol) and hydrazine (0.1 mL, excess) in ethanol (6 mL) were reacted according to General Procedure A. Product was purified using column chromatography (SiO₂, 10% methanol in methylene chloride) to yield 120 mg of the title compound (89% yield, 98.1% purity). ¹H NMR (400 MHz, DMSO-d₆) δ 8.00 (q, J=0.78, 1H), 7.78 (dd, J=8.78, 1.56, 1H), 7.53 (dd, J=8.59, 0.78, 1H), 7.29 (m, 4H), 7.19 (m, 1H), 3.84 (m, 1H), 2.92 (overlapping m, 4H), 2.72 (m, 2H), 1.32 (t, J=7.61, 3H). MS (ESI) m/z 379.6 [M+1]⁺.

B. 2-((2R)-2-{[5-(3-Ethyl(1H-indazol-5-yl))(1,3,4-thiadiazol-2-yl)]amino}-3-phenylpropyl)benzo[c]azolidine-1,3-dione. Methane sulfonic acid (1.5 mL, excess) and N-[({[(1R)-2-(1,3-dioxobenzo[c]azolidin-2-yl)-1-benzylethyl]amino}thioxomethyl)amino](3-ethyl(1H-indazol-5-yl))carboxamide (0.361 g, 0.68 mmol) were reacted as described in General Procedure B to provide pure material (0.187 g, 54%) after column chromatography (SiO₂, 2% methanol in methylene chloride). MS (ESI) m/z 509.3 [M+1]⁺.

C. N-[({[(1R)-2-(1,3-Dioxobenzo[c]azolidin-2-yl)-1-benzylethyl]amino}thioxomethyl)amino](3-ethyl(1H-indazol-5-yl))carboxamide. The hydrochloride salt of 2-((2R)-2-amino-3-phenylpropyl)benzo[c]azoline-1,3-dione (example 1.D) (0.215 g, 0.68 mmol), triethylamine (0.379 mL, 2.72 mmol), di(2-pyridyloxy)methane-1-thione (0.158 g, 0.68 mmol), and 3-ethyl-1H-indazole-5-carbohydrazide (0.139 g, 0.68 mmol) were reacted according to General Procedure C. Purification using column chromatography (SiO₂, 2% methanol in methylene chloride) provided clean product (0.361 g, 100%). MS (ESI) m/z 527.5 [M+1]⁺.

D. 3-Ethyl-1H-indazole-5-carbohydrazide. Methyl 3-ethyl-1H-indazole-5-carboxylate (0.223 g, 1.09 mmol) and hydrazine (0.1 mL, 3.28 mmol) in ethanol (3 mL) were reacted as described in General Procedure H. The resulting product was dried under high vacuum to provide the title compound as an off white solid (0.220 g, 98%). MS (ESI) m/z 205.1 [M+1]⁺.

E. Methyl 3-ethyl-1H-indazole-5-carboxylate. A solution of 3-ethyl-1H-indazole-5-carboxylic acid (0.233 g, 1.2 mmol) and sulfuric acid (0.2 mL) in methanol (5 mL) were reacted according to General Procedure I. The resulting product was purified using column chromatography (SiO₂, 7:3 n-Hexanes:ethyl acetate) to provide the title compound (0.223 g, 89%). MS (ESI) m/z 205.1 [M+1]⁺.

F. 3-Ethyl-1H-indazole-5-carboxylic acid. 3-Ethyl-1H-indazole-5-carbonitrile (0.216 g, 1.26 mmol) and HCl (3 mL) were reacted according to General Procedure J. The resulting product was purified using column chromatography (SiO₂, 10% methanol in methylene chloride) to provide the title compound (0.233 g, 97%). MS (ESI) m/z 191.2 [M+1]⁺.

G. 3-Ethyl-1H-indazole-5-carbonitrile. 4-Fluoro-3-propanoyl-benzenecarbonitrile (0.268 g, 1.51 mmol) was dissolved in toluene (2 mL) and hydrazine was added (0.1 mL, 3.33 mmol). The reaction mixture was heated in the microwave reactor at 11 0° C. for 30 min. Solvent was removed under reduced pressure and the resulting crude material was purified using column chromatography (SiO₂, 1:1 n-Hexanes:ethyl acetate) to provide the title compound (0.230 g, 89%). MS (ESI) m/z 172.1 [M+1]⁺.

H. 4-Fluoro-3-propanoylbenzenecarbonitrile. 4-Fluorobenzonitrile (1.14 g, 9.44 mmol) and N-methoxy-N-methylpropanamide (1.32 g, 11.32 mmol) were dissolved in THF (200 mL). The mixture was cooled to −78° C. and LDA (1.8 M in THF/heptane/ethylbenzene, 10.48 mL, 18.88 mmol) was added dropwise. The reaction was stirred at −78° C. for 2 hours and quenched by the addition of water. The reaction was extracted with ethyl acetate (3×100 mL), the organic layers combined and dried over magnesium sulfate. Solvent was removed under reduced pressure and the crude material was purified using column chromatography (SiO₂, 8:2 n-Hexanes:ethyl acetate) to provide the title compound (0.268 g, 16%). ¹H NMR (300 MHz, DMSO-d₆) δ 8.30 (dd, J=6.59, 2.20, 1H), 8.16 (m, 1H), 7.61 (dd, J=10.71, 8.79, 1H), 3.02 (dddd, J=7.14, 7.14, 7.14, 2.47, 2H), 1.07 (t, J=7.14, 3H).

I. N-Methoxy-N-methylpropanamide. To a solution of N,O-dimethylhydroxylamine hydrochloride (5.61 g, 57.5 mmol) and triethylamine (17.6 mL, 126.5 mmol) in methylene chloride (100 mL) was added propionyl chloride (5 mL, 57.5 mmol) dropwise. The reaction was stirred at room temperature overnight. The reaction was then washed with water (100 mL) and the organic layer was dried over magnesium sulfate. Solvent was removed under reduced pressure and the crude material was purified using column chromatography (SiO₂, n-Hexanes to 8:2 n-Hexanes:ethyl acetate) to provide the title compound (1.72 g, 25.6%). ¹H NMR (300 MHz, CD₃OD) δ 3.71 (s, 3H), 3.17 (s, 3H), 2.47 (broad m, 2H), 1.09 (t, J=7.42, 3H).

### 5.1.66 SYNTHESIS OF [(1R)-2-AMINO-1-BENZYLETHYL]{5-[3-(METHYLETHYL)(1H-INDAZOL-5-YL)](1,3,4-THIADIAZOL-2-YL)}AMINE

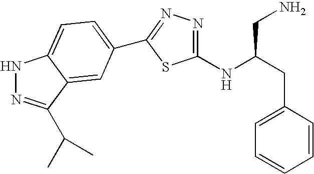

A. [(1R)-2-Amino-1-benzylethyl]{5-[3-(methylethyl)(1H-indazol-5-yl)](1,3,4-thiadiazol-2-yl)}amine. A solution of 2-[(2R)-2-({5-[3-(methylethyl)(1H-indazol-5-yl)](1,3,4-thiadiazol-2-yl)}amino)-3-phenylpropyl]benzo[c]azolidine-1,3-dione (0.256 g, 0.49 mmol) and hydrazine (0.1 mL, excess) in ethanol (6 mL) were reacted according to General Procedure A. Product was purified using column chromatography (SiO₂, 10% methanol in methylene chloride) to yield 187 mg of the title compound (97% yield, 97.3% purity). ¹H NMR (400 MHz, CD₃OD) δ 8.10 (q, J=0.78, 1H), 7.80 (dd, J=8.78, 1.56, 1H), 7.53 (dd, J=8.79, 0.78, 1H), 7.28 (m, 4H), 7.18 (m, 1H), 4.07 (m, 1H), 3.45 (m, 1H), 2.96 (m, 3H), 2.82 (dd, J=13.28, 7.81, 1H), 1.45 (d, J=7.03, 6H). MS (ESI) m/z 393.2 [M+1]⁺.

B. 2-[(2R)-2-({5-[3-(Methylethyl)(1H-indazol-5-yl)] (1,3,4-thiadiazol-2-yl)}amino)-3-phenyl propyl]benzo[c]azolidine-1,3-dione. Methane sulfonic acid (1.5 mL, excess) and N-[({[(1R)-2-(1,3-dioxobenzo[c]azolidin-2-yl)-1-benzylethyl]amino}thioxomethyl)amino][3-(methylethyl)(1H-indazol-5-yl)]carboxamide (0.267 g, 0.49 mmol) were reacted as described in General Procedure B to provide pure material (0.256 g, 99%) after column chromatography (SiO₂, 2% methanol in methylene chloride). MS (ESI) m/z 523.4 [M+1]⁺.

C. N-[({[(R)-2-(1,3-dioxobenzo[c]azolidin-2-yl)-1-benzylethyl]amino}thioxomethyl)amino][3-(methylethyl)(1H-indazol-5-yl)]carboxamide. The hydrochloride salt of 2-((2R)-2-amino-3-phenylpropyl)benzo[c]azoline-1,3-dione (0.363 g, 1.15 mmol), triethylamine (0.639 mL, 4.59 mmol), di(2-pyridyloxy)methane-1-thione (0.267 g, 1.15 mmol), and 3-(methylethyl)-1H-indazole-5-carbohydrazide (0.250 g, 1.15 mmol) were reacted according to General Procedure C. Purification using column chromatography (SiO₂, 2% methanol in methylene chloride) provided clean product (0.267 g, 43%). MS (ESI) m/z 541.5 [M+1]⁺.

D. 3-(Methylethyl)-1H-indazole-5-carbohydrazide. Methyl 3-(methylethyl)-1H-indazole-5-carboxylate (0.500 g, 2.29 mmol) and hydrazine (0.216 mL, 6.88 mmol) in ethanol (5 mL) were reacted as described in General Procedure H. The resulting product was dried under high vacuum to provide the title compound as an off white solid (0.5 g, 100%). MS (ESI) m/z 219.4 [M+1]⁺.

E. Methyl 3-(methylethyl)-1H-indazole-5-carboxylate. A solution of 3-(methylethyl)-1H-indazole-5-carboxylic acid (1 g, 4.9 mmol) and sulfuric acid (0.2 mL) in methanol (20 mL) were reacted according to General Procedure I. The resulting product was purified using column chromatography (SiO₂, 7:3 n-Hexanes:ethyl acetate) to provide the title compound (0.768 g, 72%). MS (ESI) m/z 219.4 [M+1]⁺.

F. 3-(Methylethyl-1H-indazole-5-carboxylic acid. 3-(Methylethyl)-1H-indazole-5-carbonitrile (1.17 g, 6.3 mmol) and HCl (5 mL) were reacted according to General Procedure J. The resulting product was purified using column chromatography (SiO₂, 10% methanol in methylene chloride) to provide the title compound (1 g, 78%). MS (ESI) m/z 205.1 [M+1]⁺.

G. 3-(Methylethyl)-1H-indazole-5-carbonitrile. 4-Fluoro-3-(2-methylpropanoyl)benzene carbonitrile (1.39 g, 7.28 mmol) was dissolved in toluene (8 mL) and hydrazine was added (0.5 mL, 16 mmol). The reaction mixture was heated in the microwave reactor at 110° C. for 30 min. Solvent was removed under reduced pressure and crude was purified using column chromatography (SiO₂, n-hexanes to 9:1 n-Hexanes:ethyl acetate) to provide the title compound (1.35 g, 100%). MS (ESI) m/z 186.2 [M+1]⁺.

H. 4-Fluoro-3-(2-methylpropanoyl)benzenecarbonitrile. 4-Fluorobenzonitrile (2.31 g, 19.08 mmol) and N-methoxy-2-methyl-N-methylpropanamide (3 g, 22.9 mmol) were dissolved in THF (300 mL). The mixture was cooled to −78° C. and LDA (1.8 M in THF/heptane/ethylbenzene, 23.3 mL, 41.98 mmol) was added dropwise. The reaction was stirred at −78° C. for 2 hours and quenched by the addition of water. The reaction was extracted with ethyl acetate (3×100 mL), the organic layers combined and dried over magnesium sulfate. Solvent was removed under reduced pressure and the crude material was purified using column chromatography (SiO₂, 8:2 n-Hexanes:ethyl acetate) to provide the title compound (1.39 g, 38%). ¹H NMR (300 MHz, CD₃OD) δ 8.14 (dd, J=6.59, 2.20, 1H), 7.97 (m, 1H), 7.44 (dd, J=10.44, 8.51, 1H), 3.40 (m, 1H), 1.17 (d, J=6.87, 6H).

I. N-Methoxy-2-methyl-N-methylpropanamide. To a solution of N,O-dimethyl hydroxylamine hydrochloride (5.61 g, 57.5 mmol) and triethylamine (17.6 mL, 126.5 mmol) in methylene chloride (100 mL) was added isobutyryl chloride (6.02 mL, 57.5 mmol) dropwise. The reaction was stirred at room temperature overnight. The reaction was washed with water (100 mL) and the organic layer was dried over magnesium sulfate. Solvent was removed under reduced pressure and crude purified using column chromatography (SiO₂, n-Hexanes to 9:1 n-Hexanes:ethyl acetate) to provide the title compound (5.55 g, 74%). ¹H NMR (300 MHz, DMSO-d₆) δ 3.72 (s, 3H), 3.31 (m, 1H), 3.18 (s, 3H), 1.09 (d, J=6.87, 6H).

### 5.1.67 SYNTHESIS OF [5-(3-METHYL(1H-INDAZOL-5-YL))(4H-1,2,4-TRIAZOL-3-YL)](2-PHENYLETHYL)AMINE

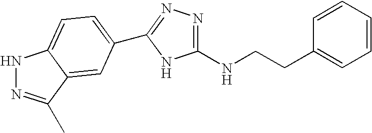

A. [5-(3-Methyl(1H-indazol-5-yl))(4H-1,2,4-triazol-3-yl)](2-phenylethyl)amine. A solution of (iminomethylthiomethyl)(2-phenylethyl)amine, hydroiodide salt, (0.186 g, 0.579 mmol) and 3-methyl-1H-indazole-5-carbohydrazide

(0.110 g, 0.579 mmol) in pyridine (2 mL) were refluxed overnight. Solvent was removed under reduced pressure and the crude material was purified using column chromatography (SiO₂, 10% methanol in methylene chloride) and reverse-phase semi-preparatory HPLC (20-100% acetonitrile+0.1% TFA in H₂O+0.1% TFA, over 20 min). Fractions containing clean product were neutralized with potassium carbonate and solvent removed under reduced pressure. The resulting material was taken up in CH₂Cl₂, washed with saturated potassium carbonate, water, and saturated sodium chloride in succession, dried over magnesium sulfate, filtered and solvent removed under reduced pressure to afford the title compound (22 mg 12% yield, 99.6% purity). ¹H NMR (400 MHz, CD₃OD) δ 8.28 (s, 1H), 7.96 (bd, J=8.79, 1H), 7.49 (bd, J=8.79, 1H), 7.29 (m, 4H), 7.20 (m, 1H), 3.57 (bt, 2H), 2.95 (t, J=7.1, 2H), 2.59 (s, 3H). MS (ESI) m/z 319.2 [M+1]⁺

B. (Iminomethylthiomethyl)(2-phenylethyl)amine, hydroiodide salt. 2-Phenylethylthiourea (0.5 g, 2.77 mmol) was dissolved in methanol (5 mL) and methyl iodide (0.172 mL, 2.77 mmol) was added. The reaction mixture was refluxed for 2 hours. Solvent was removed under reduced pressure and the resulting crude material was triturated with ethyl ether. The solid was isolated by filtration and dried to yield 0.875 g of the title product (98% yield). MS (ESI) m/z 195.2 [M+1]⁺.

### 5.1.68 SYNTHESIS OF [(1S)-2-AMINO-1-BENZYLETHYL]{5-[3-(METHYLETHYL)(1H-INDAZOL-5-YL)](1,3,4-THIADIAZOL-2-YL)}AMINE

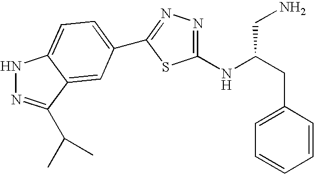

A. [(1S)-2-Amino-1-benzylethyl]{5-[3-(methylethyl)(1H-indazol-5-yl)](1,3,4-thiadiazol-2-yl)}amine. A solution of 2-[(2S)-2-({5-[3-(methylethyl)(1H-indazol-5-yl)](1,3,4-thiadiazol-2-yl)}amino)-3-phenylpropyl]benzo[c]azolidine-1,3-dione (0.380 g, 0.73 mmol) and hydrazine (0.2 mL, excess) in ethanol (6 mL) was reacted according to General Procedure A. Product was purified using column chromatography (SiO₂, 10% methanol in methylene chloride) to yield 191 mg of the title compound (67% yield, 95.6% purity). ¹H NMR (400 MHz, CD₃OD) δ8.10 (q, J=0.78, 1H), 7.80 (dd, J=8.78, 1.56, 1H), 7.53 (dd, J=8.79, 0.98, 1H), 7.28 (m, 4H), 7.18 (m, 1H), 4.04 (m, 1H), 3.45 (m, 1H), 2.94 (m, 3H), 2.79 (dd, J=13.47, 7.81, 1H), 1.45 (d, J=7.03, 6H). MS (ESI) m/z 393.4 [M+1]⁺.

B. 2-[(2S)-2-({5-[3-(Methylethyl)(1H-indazol-5-yl)](1,3,4-thiadiazol-2-yl)}amino)-3-phenyl propyl]benzo[c]azolidine-1,3-dione. Methane sulfonic acid (1 mL, excess) and N-[({[(1S)-2-(1,3-dioxobenzo[c]azolidin-2-yl)-1-benzylethyl]amino}thioxomethyl)amino][3-(methylethyl)(1H-indazol-5-yl)]carboxamide (0.523 g, 0.967 mmol) were reacted as described in General Procedure B to provide pure material (0.380 g, 75%) after column chromatography (SiO₂, 2% methanol in methylene chloride). MS (ESI) m/z 523.6 [M+1]⁺.

C. N-[({[(1S)-2-(1,3-Dioxobenzo[c]azolidin-2-yl)-1-benzylethyl]amino}thioxomethyl)amino][3-(methylethyl)(1H-indazol-5-yl)]carboxamide. The hydrochloride salt of 2-((2S)-2-amino-3-phenylpropyl)benzo[c]azoline-1,3-dione (0.385 g, 1.2 mmol), triethylamine (0.69 mL, 4.9 mmol), di(2-pyridyloxy)methane-1-thione (0.284 g, 1.2 mmol), and 3-(methylethyl)-1H-indazole-5-carbohydrazide (0.267 g, 1.2 mmol) were reacted according to General Procedure C. Purification using column chromatography (SiO₂, 2% methanol in methylene chloride) provided clean product (0.523 g, 79%). MS (ESI) m/z 541.6 [M+1]⁺.

### 5.1.69 SYNTHESIS OF [5-(3-METHYL-1H-INDAZOL-5-YL)-1,3,4-THIADIAZOL-2-YL]-PHENETHYL-AMINE

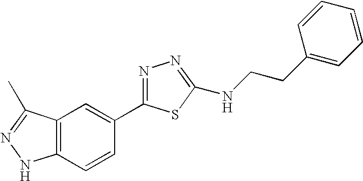

A. [5-(3-Methyl-1H-indazol-5-yl)-1,3,4-thiadiazol-2-yl]-phenethyl-amine. Methane sulfonic acid (0.80 mL, excess) and (3-methyl(1H-indazol-5-yl))-N-({[(2-phenylethyl)amino]thioxomethyl}amino)carboxamide (0.30 g, 1.6 mmol) were reacted as described in General Procedure B to provide pure material (0.072 g, 14%). ¹H NMR (400 MHz, CD₃OD) δ 8.01 (m, 1H), 7.83 (dd, 1H), 7.49 (dd, 1H), 7.25 (m, 4H), 7.17 (m, 1H). 3.63 (t, 2H), 2.96 (t, 2H), 2.55 (s, 3H); MS (ESI) m/z 336.1 [M+1]⁺; mp=175-178° C.

B. (3-Methyl(1H-indazol-5-yl))-N-({[(2-phenylethyl)amino]thioxomethyl}amino)carboxamide 3-Methyl-1H-indazole-5-carbohydrazide (0.30 g, 1.6 mmol) and phenethyl isothiocyanate (0.31 g, 1.9 mmol) were dissolved in ethanol (25 mL) and heated to 70° C. overnight. The next day the resulting off white solid was washed with methanol (20 mL) an excess H₂O. The solid was then concentrated to dryness under reduced pressure to give the desired product (0.072 g, 13%). MS (ESI) m/z 354.4 [M+1]⁺.

C. 3-Methyl-1H-indazole-5-carbohydrazide. Methyl 3-methyl-1H-indazole-5-carboxylate (0.65 g, 3.4 mmol) and hydrazine (3.0 mL, 95 mmol) in ethanol (15 mL) were reacted as described in General Procedure H. The resultant product was dried under high vacuum to provide the title compound as an off white solid (0.57 g, 3.0 mmol, 88%). MS (ESI) m/z 191.2 [M+1]⁺.

D. Methyl 3-methyl-1H-indazole-5-carboxylate. A solution of 3-Methyl-1H-indazole-5-carboxylic acid (1.0 g, 5.7 mmol) and sulfuric acid (2 mL) in methanol (100 mL) were reacted according to General Procedure I. The resulting product was dried under vacuum to provide the title compound (0.69 g, 3.6 mmol, 64%). MS (ESI) m/z 191.2 [M+1]⁺.

E. 3-Methyl-1H-indazole-5-carboxylic acid. 3-Methyl-1H-indazole-5-carbonitrile (1.5 g, 9.5 mmol) and 12M HCl (5 mL) were reacted according to General Procedure J. The resulting material was dried in a vacuum oven overnight to give the title compound as a white solid (1.1 g, 6.2 mmol, 65%). MS (ESI) m/z 177.2 [M+1]⁺.

F. 3-Methyl-1H-indazole-5-carbonitrile. Sodium nitrite (3.6 g, 53 mmol), dissolved in H₂O (2 mL), was gradually added to a solution of 4-amino-3-ethyl-benzonitrile (7.0 g, 48 mmol) in glacial acetic acid (150 mL). After 16 hours, the reaction mixture was concentrated under reduced pressure to one-third the original volume and H₂O (200 mL) was added. The aqueous phase was then adjusted to pH=10 with saturated NaHCO₃. The aqueous phase was extracted with ethyl acetate (3×200 mL) and the resulting organic extracts were combined and dried over MgSO₄. Following the concentration of the organic phase under reduced pressure the crude residue was purified using flash chromatography on silica gel (0-45% ethyl acetate in hexanes). The title compound was isolated as an orange solid (3.3 g, 21 mmol, 30%). MS (ESI) m/z 158.2 [M+1]⁺.

### 5.2 BIOLOGICAL EXAMPLES

### 5.2.1 AKT Assay (³³P Protocol)

The following assay protocol can be used to determine the AKT inhibitory activity of a Heterocyclic Compound.

The following reagents are used: Akt1 (active) (Vendor: Upstate; 0.4 mg/ml); DPHAkt1 (Vendor: Upstate; 0.1 mg/ml); DPHAkt2 (Vendor: Upstate; 0.1 mg/ml); SGK1 (Vendor: Upstate; 0.04 mg/ml); KKGGRARTSSFAEPG (substrate peptide; Vendor: Upstate; 5 mg/ml). The following reagents are combined: Test Compound/100% DMSO (5 μl/assay); AKT substrate-501 g/ml final—Stock 5 mg/ml (substrate solution at 125 μg/ml) (40 μl/assay); and enzyme solutions (Akt1 at 1.714 μg/ml, DPHAkt1 at 0.214 μg/ml, Akt2 at 0.857 μg/ml, SGK at 0.214 μg/ml) (35 μl/assay). The final concentration of DMSO in the assay is 5%.

The reaction mixture is pre-incubated for 15 min at room temperature. The assay is initiated with 20 μl kinase buffer (60 μM ATP final +1.5 μCi/reaction). The mixture is incubated at room temperature for 2 min (on shaker) and incubated for 28 min at room temperature. 100 μl of 3% H₃PO₄ (final concentration 1.5% H₃PO₄) is added. A Millipore filter plate is pre-wet with 200 μl of 0.75% H₃PO₄ and allowed to sit for 5 min. The buffer is vacuumed from the plate. 150 μl of the reaction mixture is transferred into the filter plate and the reaction is allowed to sit in the plate for 5 min before vacuuming. The plate is washed on the vacuum manifold 5X 200 μl with 0.75% H₃PO₄. The plate is placed under a heat lamp to dry. 50 μl of scintillation fluid is added and the plate is read using a liquid scintillation counter.

### 5.2.2 AKT Assay (IMAP Protocol)

The following assay protocol can be used to determine the AKT inhibitory activity of a Heterocyclic Compound.

The following reagents are used: DPHAkt1 (Vendor: Upstate; 0.1 mg/ml); 5FAM-GRPRTSSFAEG-COOH (IMAP Crosstide substrate; Vendor: Molecular Devices).

The following reagents are combined: Test Compound/20% DMSO (5 μl/assay); Crosstide substrate (400 nM solution) (100 nM final) (5 μl/assay); and enzyme solution (0.050 ng/ml) (250 pg/well final) (5 μl/assay). The final concentration of DMSO in the assay is 5%.

The reaction mixture is pre-incubated for 15 min at room temperature (1 min on shaker and 14 min in the dark). The assay is initiated with 5 μl of 65 uM ATP (final). The mixture is incubated for 1 min (on shaker) and incubated 59 min at room temperature in the dark. 60 μl of Binding Buffer is added to stop the kinase reaction. The mixture is allowed to incubate for at least 30 min in the dark for the “regular” binding buffer system (1× buffer 1:400 beads). The mixture is allowed to incubate for at least 2 hours when using Progressive buffer binding system (50% A and 50% B 1:700 Beads). The fluorescent polarization is read using an Analyst instrument.

The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

A number of references have been cited, the disclosures of which are incorporated herein by reference in their entirety.

What is claimed is:

1. A compound having the formula: 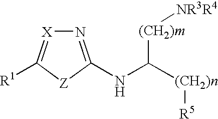
or a pharmaceutically acceptable salt thereof, wherein:
X is N or CH;
Z is NH or S;
R¹ is substituted or unsubstituted C₃₋₁₀heteroaryl;
R³ and R⁴ are independently H, substituted or unsubstituted C₁₋₈alkyl, substituted or unsubstituted aryl or substituted or unsubstituted C₃₋₁₀heteroaryl;
R⁵ is substituted or unsubstituted aryl or substituted or unsubstituted C₃₋₁₀heteroaryl;
m is an integer from 1-3; and
n is an integer from 0-3.

2. A compound of claim 1, wherein X is N.

3. A compound of claim 1, wherein Z is S.

4. A compound of claim 1, wherein X is N and Z is S.

5. A compound of claim 1, wherein R¹ is substituted C₃₋₁₀heteroaryl.

6. A compound of claim 1, wherein R³ and R⁴ are both H.

7. A compound of claim 1, wherein m and n are both 1.

8. A compound of claim 1, wherein X is N, Z is S, R³ and R⁴ are both H, and m and n are both 1.

9. A compound having the formula: 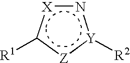
or a pharmaceutically acceptable salt thereof, wherein:
X is N or CH;
Y is N or C;
Z is CH or S;
R¹ is substituted or unsubstituted C₃₋₁₀heteroaryl; and
R² is substituted or unsubstituted C₁₋₈ alkyl.

10. A compound of claim 9, wherein X is N and Y is N.

11. A compound of claim 9, wherein Z is CH.

12. A compound of claim 9, wherein Y is N and Z is CH.

13. A compound of claim 9, wherein R¹ is substituted C₃₋₁₀heteroaryl.

14. A compound of claim 9, wherein R² is substituted C₁₋₈ alkyl.

15. A method for treating or preventing cancer, an inflammatory condition, an immunological condition or a metabolic condition comprising administering to a patient in need thereof an effective amount of a compound of claim 1.

16. The method of claim 15, wherein the cancer is of the head, neck, eye, mouth, throat, esophagus, bronchus, larynx, pharynx, chest, bone, lung, colon, rectum, stomach, prostate, urinary bladder, uterine, cervix, breast, ovaries, testicles, skin, thyroid, blood, lymph nodes, kidney, liver, pancreas, brain or central nervous system.

17. The method of claim 15, wherein the inflammatory condition is asthma, allergic rhinitis, bronchitis, chronic obstructive pulmonary disease, cystic fibrosis, inflammatory bowel disease, irritable bowel syndrome, Crohn's disease, mucous colitis, ulcerative colitis, diabetes or obesity.

18. The method of claim 15, wherein the immunological condition is rheumatoid arthritis, rheumatoid spondylitis, osteoarthritis, multiple sclerosis, lupus, inflammatory bowel disease, ulcerative colitis, Crohn's disease, myasthenia gravis, Grave's disease or diabetes.

19. The method of claim 15, wherein the metabolic condition is obesity or diabetes.

20. A pharmaceutical composition comprising a compound of claim 1, and a pharmaceutically acceptable carrier, excipient or diluent.

21. The pharmaceutical composition of claim 20 suitable for oral, parenteral, mucosal, transdermal or topical administration.

22. The pharmaceutical composition of claim 20 suitable for oral administration.

23. A single unit dosage form comprising a compound of claim 1, and a pharmaceutically acceptable carrier, excipient or diluent.

24. The single unit dosage form of claim 23 suitable for oral, parenteral, mucosal, transdermal or topical administration.

25. The single unit dosage form of claim 23 suitable for oral administration.
